(19) United States
(12) Patent Application Publication
(10) Pub. No.: US 2024/0417359 A1
(43) Pub. Date: Dec. 19, 2024

(54) PRECURSORS OF PHENOLIC FRAGRANT COMPOUNDS

(71) Applicant: GIVAUDAN SA, VERNIER (CH)

(72) Inventors: Nicolas COCITO ARMANlNO, KKemptthal (CH); An CHAI, Shanghai (CN); Felix FLACHSMANN, Kemptthal (CH); Marc LINIGER, Kemptthal (CH); Lijun ZHOU, Shanghai (CN)

(21) Appl. No.: 18/703,358

(22) PCT Filed: Oct. 20, 2022
(86) PCT No.: PCT/EP2022/079166

§ 371 Date: Apr. 21, 2024

(30) Foreign Application Priority Data
Oct. 21, 2021 (WO) PCT/CN2021/125209

(51) Int. Cl. C07C 51/347; C11B 9/00

(52) U.S. Cl. CPC C07C 51/347; C11B 9/0061

(57) ABSTRACT

There is provided a compound of formula (I)

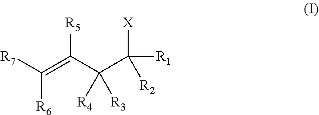

which is a homoallylic ether of a phenolic fragrant compound HX and which is able to release said phenolic fragrant compound HX and optionally a further fragrant.

### TECHNICAL FIELD

The present invention relates generally to fragrance precursors, which are able to release phenolic fragrant compounds. The invention also relates to perfume preparations and consumer products containing said precursors. The invention further relates to methods of making said perfume precursors, perfume preparations and consumer products, as well as the use of said perfume precursors and perfume preparations in consumer products, such as personal care and household care products.

### BACKGROUND

Perfumed consumer products such as cleaning or laundry products comprising fragrances are well-known in the art. However, it is known that fragrances can be altered when incorporated in certain consumer product bases, where alkalinity, acidity, the presence of oxidizing agents, such as hypochlorite salts, or other base components may lead to chemical degradation of the fragrance. In addition, volatile fragrances tend to be dissipated with time. Furthermore, when used in cleaning or laundry products, the deposition of the fragrance on a treated substrate is diminished by the washing and/or rinsing procedure.

In addition, in case of phenolic fragrant compounds like vanillin or ethyl vanillin, discoloration issues in media like detergent bases can occur. Furthermore, phenolic fragrant compounds show a low substantivity in rinse-off applications due to their relatively high water solubility.

Nevertheless, it is desired by consumers to have products that can be stored over time and still giving a constant perfume impression without discoloration. In particular, the impact of volatile components is to be retained. Furthermore, it is desired that such products create a long-lasting pleasant fragrance slowly emitting from the treated substrate over time.

To address these needs, fragrance precursors can be used, which are substances that are essentially odorless themselves, but which, in particular circumstances, will decompose to release the fragrant molecule.

There are several classes of known precursors which release fragrant molecules upon activation, such as hydrolysis, temperature change, oxygen, action of light and enzymes. For example, WO2012085287 reports a group of precursors able to release a fragrance by spontaneous air oxidation. In WO2007143873 another group of precursors is described which can be cleaved by hydrolysis.

All precursors do show different stability, and they release the fragrant molecule under different conditions. It is therefore desirable to provide a new or improved system which is capable of releasing phenolic fragrant compounds with significant substantivity while avoiding discoloration.

### SUMMARY

In accordance with a first aspect of the present invention there is provided a use of a compound as fragrance precursor which is capable of releasing phenolic fragrant compounds and optionally further fragrant compounds.

In accordance with a second aspect of the present invention there are provided compounds as fragrance precursors.

In accordance with a third aspect of the present invention there are provided fragrance compositions and consumer products comprising said fragrance precursors.

In accordance with a fourth aspect of the present invention there is provided a method to release a phenolic fragrant compound and optionally further fragrant compounds.

In accordance with a fifth aspect of the present invention there is provided a method of making said fragrance precursors, and fragrance compositions and consumer products comprising said fragrance precursors.

In accordance with a sixth aspect of the present invention there is provided a method to confer, enhance, improve or modify the hedonic properties of a fragrance composition or a consumer product by using said fragrance precursors.

Certain embodiments of any aspect of the present invention may provide one or more of the following advantages:
release of phenolic fragrant compounds,
optionally release of further fragrant compounds,
stability of fragrance precursors in consumer product bases as well as after their deposition on substrates,
avoidance or great reduction of discoloration effects in comparison to accords using the free phenols.

The details, examples and preferences provided in relation to any particular one or more of the stated aspects of the present invention will be further described herein and apply equally to all aspects of the present invention. Any combination of the embodiments, examples and preferences described herein in all possible variations thereof is encompassed by the present invention unless otherwise indicated herein, or otherwise clearly contradicted by context.

### DETAILED DESCRIPTION

The present invention is based on the surprising finding that homoallyl ethers of phenolic fragrant compounds can serve as fragrance precursors able to release said phenolic fragrant compounds. These fragrance precursors have a higher substantivity than the phenolic fragrant compounds while avoiding discoloration issues typically occurring when using free phenolic fragrant compounds.

There is therefore provided herein a use of a compound of formula (I)

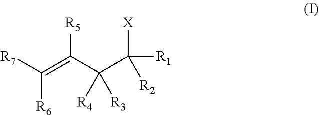

wherein
R₁-R₇ is independently selected from the group consisting of H, Me or linear or branched C2-C20 hydrocarbon groups, bearing up to 4 heteroatoms selected from the group consisting of O, S, and N, and bearing up to three double bonds or two triple bonds, and bearing up to three rings, phenyl, benzyl, substituted aryl or hetero-aryl units, 1- or 2-naphthyl,
or R₆ and R₇ form together a double bond to a carbon atom, which is bearing one or two C1-C10 alkyl or C2-C10 alkenyl substituents, while the other substituents have the same meaning as previously defined;
or R₁ and R₆, R₁ and R₃, R₁ and R₅, R₃ and R₅, R₃ and R₆, R₅ and R₇, R₆ and R₇, R₁ and R₂, or R₃ and R₄, form together with the carbon atoms of the chain they are attached to a 5 or 6 membered ring bearing up to 2 heteroatoms selected from the group consisting of O, S and N, that is optionally substituted with an alkyl chain, while the other substituents have the same meaning as previously defined;
and the sum of all carbon atoms of R₁-R₇ is at least 5 or more;
or R₇ is —CR′₃R′₄—CR′₁R′₂—X′, wherein R′₁, R′₂, R′₃, R′₄, and X′ can be selected from the corresponding groups of R₁, R₂, R₃, R₄, and X, while the other substituents have the same meaning as previously defined, and the sum of all carbon atoms of R₁-R₆ and R′₁-R′₄ is at least 5 or more;
and wherein X is representing the following fragment

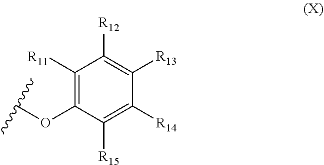

wherein
R₁₁-R₁₅ is independently selected from the group consisting of H, linear or branched C1-C5 alkyl, linear or branched C2-C5 alkenyl, linear or branched C2-C5 alkynyl, OH, C1-C4-alkoxy, CHO, C(O)Me, C(O)Et, hexanoyl, heptanoyl, octanoyl, propanoyl, 3-phenylpropanoyl, 5-methylhexanoyl, (CH₂)₂C(O)CH₃, (4-methyl-3,6-dihydro-2H-pyran-2-yl), CH₂—O-Me, CH₂—O-Et, CH₂—O-iPr, CH₂—OH, Cl, Br, phenyl, CH(OR₁₆)(OR₁₇) wherein R₁₆ and R₁₇ are independently selected from the group consisting of C1-C5 alkyl or R₁₆ and R₁₇ form together with the carbon atoms of the chain they are attached to a 5 or 6 membered ring, C═N—OR₁₈ wherein R₁₈ is C1-C5 alkyl, CO₂R₁₉ wherein R₁₉ is selected from the group consisting of linear or branched C1-C8 alkyl, linear or branched C2-C8 alkenyl, linear or branched C2-C8 alkynyl, cycloalkyl and aryl;
or R₁₁ and R₁₂ or R₁₂ and R₁₃ may form together with the carbon atoms they are attached to C1-C5 alkyl substituted or unsubstituted, saturated or unsaturated 5 and 6 membered rings containing C, O and/or N atoms;
as precursor for generating a phenolic fragrant compound HX which is a compound of formula (II)

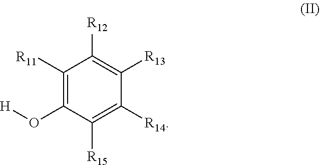

For example, there is provided a use of a compound of formula (I)

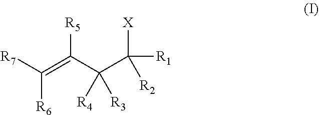

wherein
R₁-R₇ is independently selected from the group consisting of H, Me, C2-C20 branched or linear alkyl or alkenyl or alkynyl, cycloalkyl, cycloalkenyl, phenyl, benzyl, phenylethyl or substituted phenylethyl, substituted aryl or hetero-aryl units, 1- or 2-naphthyl, alkoxycarbonylates (—RO(C═O)—R′), alkylcarboxylates (—R(C═O)—O—R′), ethers, aldehydes, ketones, alcohols;
or R₆ and R₇ form together a double bond to a carbon atom, which is bearing one or two C1-C10 alkyl or C2-C10 alkenyl substituents, while the other substituents have the same meaning as previously defined;
or R₁ and R₆, R₁ and R₃, R₁ and R₅, R₃ and R₅, R₃ and R₆, R₅ and R₇, R₆ and R₇, R₁ and R₂, or R₃ and R₄, form together with the carbon atoms of the chain they are attached to a 5 or 6 membered ring bearing up to 2 heteroatoms selected from the group consisting of O, S and N, that is optionally substituted with an alkyl chain, while the other substituents have the same meaning as previously defined;
and the sum of all carbon atoms of R₁-R₇ is at least 5 or more;
or R₇ is —CR′₃R′₄—CR′₁R′₂—X′, wherein R′₁, R′₂, R′₃, R′₄, and X′ can be selected from the corresponding groups of R₁, R₂, R₃, R₄, and X, while the other substituents have the same meaning as previously defined, and the sum of all carbon atoms of R₁-R₆ and R′₁-R′₄ is at least 5 or more;
and wherein X is representing the following fragment

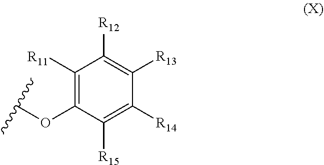

wherein
R₁₁ is selected from the group consisting of H, C1-C5 alkyl, OH, OMe, OEt, O-n-Pr, O-iso-Pr, hexanoyl, heptanoyl, octanoyl, propanoyl, 3-phenylpropanoyl, 5-methylhexanoyl, Cl, Br, phenyl, CO₂R₁₉, wherein R₁₉ is selected from the group consisting of H, Me, Et, 2-methylpropyl, hexan-2-yl, 2-isopropoxyethyl, 3-methylbut-2-en-1-yl, n-pentyl, hexyl, benzyl, cyclohexyl, cis-3-hexenyl, 2-ethylhexyl, 3-methylhex-2-en-1-yl, 2-hept-4-enyl, 1-(3-methyl-2-hexenyl), (CH₂)₂-phenyl, para-tolyl;
R₁₂ is selected from the group consisting of H, OH, C1-C5 alkyl, C2-C5 alkenyl, C1-C5 alkoxy;
R₁₃ is selected from the group consisting of H, branched or linear C1-C5 alkyl, branched or linear C2-C5 alkenyl, CHO, CO₂H, CO₂Me, CO₂Et, C(O)Me, C(O)Et, (CH₂)₂C(O)CH₃; (4-methyl-3,6-dihydro-2H-pyran-2-yl), CH₂—O-Me, CH₂—O-Et, CH₂—O-iPr, CH₂—OH, Cl, propanoyl, CO₂R₁₉, wherein R₁₉ is selected from the group consisting of H, Me, Et, 2-methylpropyl, hexan-2-yl, 2-isopropoxyethyl, 3-methylbut-2-en-1-yl, n-pentyl, hexyl, benzyl, cyclohexyl, cis-3-hexenyl, 2-ethylhexyl, 3-methylhex-2-en-1-yl, 2-hept-4-enyl, 1-(3-methyl-2-hexenyl), (CH₂)₂-phenyl, para-tolyl
R₁₄ is selected from the group consisting of H, OH, C1-C5 alkyl, C2-C5 alkenyl, C1-C5 alkoxy;
R₁₅ is selected from the group consisting of H, C1-C5 alkyl, Br,
or R₁₁ and R₁₂ or R₁₂ and R₁₃ may form together with the carbon atoms they are attached to C1-C5 alkyl substituted or unsubstituted, saturated or unsaturated 5 and 6 membered rings containing C, O and/or N atoms,
as precursor for generating a phenolic fragrant compound HX which is a compound of formula (II)

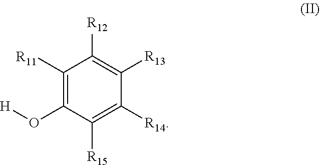

For example, in the compounds of formula (I) as described above, the sum of all carbon atoms of R₁-R₇ is at least 5 or more. In particular, the sum of all carbon atoms of R₁-R₇ is at least 5 and up to 20, more particular 5 to 15, even more particular 5 to 12, or 5 to 10.

If not further specified, the CC double bond in the compound of formula (I) has either E- or Z-configuration, or the compound is a mixture of E and Z isomers.

The compounds of formula (I) can have one or more stereo centers and in consequence exist as different stereoisomers (e.g. diastereomers or enantiomers), all of which are encompassed by the present invention. Chiral compounds of formula (I) can be enantiomerically pure, enriched or racemic. If more than one stereo center is present, the compounds are either diastereomeric mixtures, diastereomerically enriched or pure diastereomers.

The precursor effect of homoallyl ethers (compounds of formula (I)) is surprising, since allyl ethers and ethers with saturated side chains deliver significantly inferior performance.

The compound of formula (I) typically has low odor, when freshly prepared, and is able to release phenolic fragrant compound HX of formula (II). Typically, it is able to further release the residue that was attached to the fragment X or one or more fragments thereof. The residue or its fragment typically possess an own odor and therefore contributes to the overall odor impression when the compound of formula (I) is cleaved.

By the use of the compound of formula (I) instead of the phenolic fragrant compound HX as such, it is possible to provide the odor of the phenolic fragrant compound HX, optionally in combination with the odor of the released residue or its fragment, while avoiding discoloration issues in a consumer product and/or on the surface, on which the consumer product is applied.

In addition, by the use of the compound of formula (I) instead of the phenolic fragrant compound HX as such, it is possible to provide the odor of the phenolic fragrant compound HX with an enhanced intensity, optionally in combination with the odor of the released residue or its fragment, as the precursor remain on a surface, where it was deposited until the release of the phenolic fragrant compound HX and residue. In contrast, the phenolic fragrant compound HX is for example washed away in rinse off applications due to the high water solubility of the compound.

The term C2-C20 branched or linear alkyl means all linear or branched alkyl chains having between 2 and 20 carbon atoms, for example ethyl, propyl, butyl, pentyl, hexyl, heptyl, octyl, nonyl, decyl etc.

The term C2-C20 branched or linear alkenyl means all linear or branched alkenyl chains having between 2 and 20 carbon atoms and one or more double bonds, for example vinyl, allyl, prop-1-en-2-yl, prop-1-en-1-yl, prop-2-en-2-yl, pent-2-en-1-yl, pent-2-en-2-yl, hex-3-en-1-yl, oct-1-en-1-yl, 8-methylnona-3,7-dien-1-yl, 6-methylhept-5-en-2-yl, dec-9-en-1-yl etc.

The term cycloalkyl or cycloalkenyl means cyclic alkyl or alkenyl residues, optionally substituted with C1-C5 alkyl or C2-C5 alkenyl residues as defined above.

In addition, the alkyl and alkenyl residues can bear methyl or ethyl ester groups.

For example, there is provided the use of the compound of formula (I) as precursor for generating a phenolic fragrant compound HX, wherein the residue attached to the fragment X is selected from the group consisting of cis or trans 3,6-nonadienyl, 4-methyl-3-nonenyl, 4-dec-1-enenyl, 6-pentadec-3-enyl, 4-deca-1,7-dienyl.

There is further provided the use of the compound of formula (I) as precursor for generating a phenolic fragrant compound HX, wherein the residue attached to the fragment X is selected from the group consisting of 3-oxo-2-oxa-eicosa-11-en-14-yl, methyl 12-octadec-9-enyloate, 5-(2-methylundec-2-enyl), 4-(5-methylocta-1,5-dienyl), 1-phenylbut-3-enyl, 7-octadeca-9,11-dienyl, 7,12-(octadec-9-enediyl), 4-nona-1,6-dienyl, 1-(5-methyl-2-(prop-1-en-2-yl)cyclohexanyl), 4-(1-phenylnon-1-enyl), 1-(2,2,5-trimethylhexa-3,4-dienyl), 6-undec-3-enyl, 1-(2,2,5,9-tetramethyldeca-3,4,8-trienyl), 4-non-6-enyl, 1-(4,8,12-trimethyltrideca-3,7,11-trienyl), 1-(2-benzyl-2-methylpent-3-enyl), 4-tetradec-1-enyl, 4-tridec-1-enyl, 4-tetradec-1-enyl, 2-(ethyl hept-4-enyloate), 6-undeca-3,8-dienyl, dodeca-3,9-dien-1-yl, deca-1,7-dien-4-yloxy, (6-(benzo[d][1,3]dioxol-5-yl)-4-methylhex-3-en-1-yl, 4-methyl-6-(2,6,6-trimethylcyclohex-1-en-1-yl)hex-3-en-1-yl, 4-methyl-6-(2,6,6-trimethylcyclohex-1-en-1-yl)hexa-3,5-dien-1-yl, 5,9-dimethyldeca-3,8-dien-1-yl, 6-phenylhex-3-en-1-yl, 6-(4-isobutylphenyl)hex-3-en-1-yl, 3-(2-(2-(4-methylcyclohex-3-en-1-yl)propyl)cyclopentylidene) propyl, 4-cyclopentylpent-3-en-1-yl, 4-methyldec-3-en-1-yl, 4-methyldodec-3-en-1-yl, tridec-1-en-4-yl, 4-(tetradeca-1,13-dien-4-yl or is derived from the triglyceride of ricinoleic acid.

According to another aspect of the invention, there is provided the use of the compound of formula (I) as precursor for generating a phenolic fragrant compound HX of formula (II) that can be released from the compound of formula (I), wherein the fragment X is derived from a phenolic fragrant compound HX selected from the group consisting of Vanillin (4-hydroxy-3-methoxybenzaldehyde), Ethyl vanillin (3-ethoxy-4-hydroxybenzaldehyde), Eugenol (4-allyl-2-methoxyphenol), Isoeugenol ((E)-2-methoxy-4-(prop-1-en-1-yl)phenol), Raspberry ketone (4-(4-hydroxyphenyl)butan-2-one), Evernyl (methyl 2,4-dihydroxy-3,6-dimethylbenzoate), Seamoss (methyl 2,4-dihydroxy-3-methylbenzoate), Cresol (2-methylphenol, 3-methylphenol, or 4-methylphenol), 4-ethyl phenol, 3-propyl phenol, Ultravanil (2-ethoxy-4-methylphenol), Guaiacol (2-Methoxyphenol), Vinyl Guaiacol (4-Ethenyl-2-methoxyphenol), Methyl Diantilis (2-Ethoxy-4-(methoxymethyl)phenol), Thymol (5-Methyl-2-(propan-2-yl)phenol), Eugewhite (2-methoxy-4-(4-methyl-3,6-dihydro-2H-pyran-2-yl)phenol), Zingerone (4-(4-hydroxy-3-methoxyphenyl)butan-2-one), Karmaflor ((4Z)-hept-4-en-2-yl 2-hydroxybenzoate), Saffiano (3-methylbenzofuran-5-ol), Carvacrol (5-isopropyl-2-methylphenol), Creosol (2-methoxy-4-methylphenol), Orcinyl 3 (3-methoxy-5-methylphenol), Hexyl salicylate (hexyl 2-hydroxybenzoate), Cyclohexyl salicylate (cyclohexyl 2-hydroxybenzoate), Phenyl ethyl salicylate (phenethyl 2-hydroxybenzoate), Octyl salicylate (2-ethylhexyl 2-hydroxybenzoate), Yasminate (3-methylhex-2-en-1-yl 2-hydroxybenzoate), Ethyl salicylate (ethyl 2-hydroxybenzoate), Hexenyl-3-cis salicylate ((Z)-hex-3-en-1-yl 2-hydroxybenzoate), Isobutyl salicylate (isobutyl 2-hydroxybenzoate), Methyl salicylate (methyl 2-hydroxybenzoate), Vanitrop (2-ethoxy-5-(prop-1-en-1-yl)phenol), Methylpentyl salicylate (hexan-2-yl 2-hydroxybenzoate), Para-cresyl salicylate (p-tolyl 2-hydroxybenzoate), Prenyl salicylate (3-methylbut-2-en-1-yl 2-hydroxybenzoate), Amyl salicylate (pentyl 2-hydroxybenzoate), Benzyl salicylate (benzyl 2-hydroxybenzoate), Sakura salicylate (2-isopropoxyethyl 2-hydroxybenzoate), 3-propylphenol, 4-ethylphenol, 2-ethoxyphenol, 1-(2-hydroxyphenyl)-3-phenylpropan-1-one, 1-(2-hydroxyphenyl)hexan-1-one, 1-(2-hydroxyphenyl)heptan-1-one.

According to one aspect of the invention, there is provided the use of the compound of formula (I) as precursor for generating a phenolic fragrant compound HX of formula (II) that can be released from the compound of formula (I), wherein the fragment X is derived from a phenolic fragrant compound HX selected from the group consisting of Vanillin (4-hydroxy-3-methoxybenzaldehyde), Ethyl vanillin (3-ethoxy-4-hydroxybenzaldehyde), Eugenol (4-allyl-2-methoxyphenol), Isoeugenol ((E)-2-methoxy-4-(prop-1-en-1-yl)phenol), Raspberry ketone (4-(4-hydroxyphenyl)butan-2-one), Evernyl (methyl 2,4-dihydroxy-3,6-dimethylbenzoate), Seamoss (methyl 2,4-dihydroxy-3-methylbenzoate), Cresol (2-methylphenol, 3-methylphenol, or 4-methylphenol), 4-ethyl phenol, 3-propyl phenol, Ultravanil (2-ethoxy-4-methylphenol), Guaiacol (2-Methoxyphenol), Vinyl Guaiacol (4-Ethenyl-2-methoxyphenol), Methyl Diantilis (2-Ethoxy-4-(methoxymethyl)phenol), Thymol (5-Methyl-2-(propan-2-yl)phenol), Eugewhite (2-methoxy-4-(4-methyl-3,6-dihydro-2H-pyran-2-yl)phenol), Zingerone (4-(4-hydroxy-3-methoxyphenyl)butan-2-one), Karmaflor ((4Z)-hept-4-en-2-yl 2-hydroxybenzoate), Saffiano (3-methylbenzofuran-5-ol), Carvacrol (5-isopropyl-2-methylphenol), Creosol (2-methoxy-4-methylphenol), Orcinyl 3 (3-methoxy-5-methylphenol), Hexyl salicylate (hexyl 2-hydroxybenzoate), Cyclohexyl salicylate (cyclohexyl 2-hydroxybenzoate), Phenyl ethyl salicylate (phenethyl 2-hydroxybenzoate), Octyl salicylate (2-ethylhexyl 2-hydroxybenzoate), Yasminate (3-methylhex-2-en-1-yl 2-hydroxybenzoate), Ethyl salicylate (ethyl 2-hydroxybenzoate), Hexenyl-3-cis salicylate ((Z)-hex-3-en-1-yl 2-hydroxybenzoate), Isobutyl salicylate (isobutyl 2-hydroxybenzoate), Methyl salicylate (methyl 2-hydroxybenzoate), Vanitrop (2-ethoxy-5-(prop-1-en-1-yl)phenol), Methylpentyl salicylate (hexan-2-yl 2-hydroxybenzoate), Para-cresyl salicylate (p-tolyl 2-hydroxybenzoate), Prenyl salicylate (3-methylbut-2-en-1-yl 2-hydroxybenzoate), Amyl salicylate (pentyl 2-hydroxybenzoate), Benzyl salicylate (benzyl 2-hydroxybenzoate), Sakura salicylate (2-isopropoxyethyl 2-hydroxybenzoate), 3-propylphenol, 4-ethylphenol, 2-ethoxyphenol, 1-(2-hydroxyphenyl)-3-phenylpropan-1-one, 1-(2-hydroxyphenyl)hexan-1-one, 1-(2-hydroxyphenyl)heptan-1-one, and wherein the residue attached to the fragment X is selected from the group consisting of cis or trans 3,6-nonenyl, 4-methyl-3-nonenyl, 4-dec-1-enenyl, 6-pentadec-3-enyl, 4-deca-1,7-dienyl.

For example, there is provided the use of the compound of formula (I) as precursor selected from the group consisting of methyl (S,Z)-12-(2-ethoxy-4-formylphenoxy)octadec-9-enoate, methyl (S,Z)-12-(4-(3-oxobutyl)phenoxy)octadec-9-enoate, methyl (S,Z)-2-hydroxy-4-((18-methoxy-18-oxooctadec-9-en-7-yl)oxy)-3,6-dimethylbenzoate, 3-ethoxy-4-((2-methylundec-2-en-5-yl)oxy)benzaldehyde, 4-(4-((5-methylocta-1,5-dien-4-yl)oxy)phenyl)butan-2-one, 2-methoxy-1-((5-methylocta-1,5-dien-4-yl)oxy)-4-(prop-1-en-1-yl)benzene, methyl 2-hydroxy-3,6-dimethyl-4-((5-methylocta-1,5-dien-4-yl)oxy)benzoate, 4-(4-((1-phenylbut-3-en-1-yl)oxy)phenyl)butan-2-one, 4-(4-((octadeca-9,11-dien-7-yl)oxy)phenyl)butan-2-one, 4,4′-((octadec-9-ene-7,12-diylbis(oxy))bis(4,1-phenylene))bis(butan-2-one), 3-ethoxy-4-((5-methylocta-1,5-dien-4-yl)oxy)benzaldehyde, (Z)-4-(deca-1,7-dien-4-yloxy)-3-ethoxybenzaldehyde, 2-methoxy-1-(((Z)-nona-1,6-dien-4-yl)oxy)-4-(prop-1-en-1-yl)benzene, 4-(4-(((1S,2S,5R)-5-methyl-2-(prop-1-en-2-yl)cyclohexyl)oxy)phenyl)butan-2-one, 3-ethoxy-4-((1-phenylnon-1-en-4-yl)oxy)benzaldehyde, 4-((2,6-dimethylhepta-4,5-dien-2-yl)oxy)-3-ethoxybenzaldehyde, (Z)-3-ethoxy-4-(undec-3-en-6-yloxy)benzaldehyde, 3-ethoxy-4-((2,6,10-trimethylundeca-4,5,9-trien-2-yl)oxy)benzaldehyde, (E)-3-ethoxy-4-(non-6-en-4-yloxy)benzaldehyde, 3-ethoxy-4-(((3E,7E)-4,8,12-trimethyltrideca-3,7,11-trien-1-yl)oxy)benzaldehyde, 3-ethoxy-4-((2-methyl-1-phenylhex-4-en-2-yl)oxy)benzaldehyde, 4-(dec-1-en-4-yloxy)-3-ethoxybenzaldehyde, 4-(4-(dec-1-en-4-yloxy)phenyl)butan-2-one, methyl 4-(dec-1-en-4-yloxy)-2-hydroxy-3,6-dimethylbenzoate, methyl 2-(tetradec-1-en-4-yloxy)benzoate, 1-(2-(tetradec-1-en-4-yloxy)phenyl)propan-1-one, 3-ethoxy-4-(tetradec-1-en-4-yloxy)benzaldehyde, (Z)-3-ethoxy-4-(pentadec-3-en-6-yloxy)benzaldehyde, (E)-3-ethoxy-4-(pentadec-3-en-6-yloxy)benzaldehyde, 3-ethoxy-4-(((3Z,6Z)-nona-3,6-dien-1-yl)oxy)benzaldehyde, 4-(4-(((3Z,6Z)-nona-3,6-dien-1-yl)oxy)phenyl)butan-2-one, 1-methoxy-2-(((3Z,6Z)-nona-3,6-dien-1-yl)oxy)benzene, 2-(((3Z,6Z)-nona-3,6-dien-1-yl)oxy)naphthalene, 3-methoxy-4-(((3Z,6Z)-nona-3,6-dien-1-yl)oxy)benzaldehyde, 3-methoxy-4-(tridec-1-en-4-yloxy)benzaldehyde, 4-(4-(tridec-1-en-4-yloxy)phenyl)butan-2-one, 2-ethoxy-4-(methoxymethyl)-1-(tridec-1-en-4-yloxy)benzene, 3-methoxy-4-(tetradec-1-en-4-yloxy)benzaldehyde, 4-(4-(pentadec-3-en-6-yloxy)phenyl)butan-2-one, ethyl 2-(2-ethoxy-4-formylphenoxy)hept-4-enoate, 3-ethoxy-4-(((3Z,8Z)-undeca-3,8-dien-6-yl)oxy)benzaldehyde, 3-ethoxy-4-(((3Z,8Z)-undeca-3,8-dien-6-yl)oxy)benzaldehyde, 3-ethoxy-4-((4-methylnon-3-en-1-yl)oxy)benzaldehyde, 3-ethoxy-4-(undec-2-en-5-yloxy)benzaldehyde, 4-(dodec-3-en-6-yloxy)-3-ethoxybenzaldehyde, methyl (Z)-12-(2-methoxyphenoxy)octadec-9-enoate, 1-(dec-1-en-4-yloxy)-2-ethoxy-4-methylbenzene, 2-(dec-1-en-4-yloxy)-1-ethoxy-4-methylbenzene, 3-ethoxy-4-((4-methyl-6-phenylhex-3-en-1-yl)oxy)benzaldehyde, (E)-3-ethoxy-4-((4-phenylpent-3-en-1-yl)oxy)benzaldehyde, 3-ethoxy-4-((4-(4-methoxyphenyl)pent-3-en-1-yl)oxy)benzaldehyde, 3-ethoxy-4-((4-methyl-5-phenylpent-3-en-1-yl)oxy)benzaldehyde, (E)-3-ethoxy-4-((4-(naphthalen-2-yl)pent-3-en-1-yl)oxy)benzaldehyde, 4-(4-((4-methyl-6-phenylhex-3-en-1-yl)oxy)phenyl)butan-2-one, 4-(4-((4-(4-methoxyphenyl)pent-3-en-1-yl)oxy)phenyl)butan-2-one, 3-ethoxy-4-((6-(4-methoxyphenyl)-4-methylhex-3-en-1-yl)oxy)benzaldehyde, 4-(4-((6-(4-methoxyphenyl)-4-methylhex-3-en-1-yl)oxy)phenyl)butan-2-one, methyl 2-((4-methyl-6-phenylhex-3-en-1-yl)oxy)benzoate, 1-(2-((4-methyl-6-phenylhex-3-en-1-yl)oxy)phenyl)propan-1-one, 3-ethoxy-4-((4-methyl-7-phenylhept-3-en-1-yl)oxy)benzaldehyde, 4-(4-((4-methyl-7-phenylhept-3-en-1-yl)oxy)phenyl)butan-2-one, 4-(((3E,9Z)-dodeca-3,9-dien-1-yl)oxy)-3-ethoxybenzaldehyde, methyl 4-(((3E,9Z)-dodeca-3,9-dien-1-yl)oxy)-2-hydroxy-3,6-dimethylbenzoate, 4-(deca-1,7-dien-4-yloxy)-3-ethoxybenzaldehyde, 4-((6-(benzo[d][1,3]dioxol-5-yl)-4-methylhex-3-en-1-yl)oxy)-3-ethoxybenzaldehyde, 4-(4-((6-(benzo[d][1,3]dioxol-5-yl)-4-methylhex-3-en-1-yl)oxy)phenyl)butan-2-one, 3-ethoxy-4-((4-methyl-6-(2,6,6-trimethylcyclohex-1-en-1-yl)hex-3-en-1-yl)oxy)benzaldehyde, 4-(4-((4-methyl-6-(2,6,6-trimethylcyclohex-1-en-1-yl)hex-3-en-1-yl)oxy)phenyl)butan-2-one, 3-ethoxy-4-((4-methyl-6-(2,6,6-trimethylcyclohex-1-en-1-yl)hexa-3,5-dien-1-yl)oxy)benzaldehyde, 4-((5,9-dimethyldeca-3,8-dien-1-yl)oxy)-3-ethoxybenzaldehyde, 3-ethoxy-4-((6-phenylhex-3-en-1-yl)oxy)benzaldehyde, 3-ethoxy-4-((6-(4-isobutylphenyl)hex-3-en-1-yl)oxy)benzaldehyde, 3-ethoxy-4-(3-(2-(2-(4-methylcyclohex-3-en-1-yl)propyl)cyclopentylidene) propoxy)benzaldehyde, 4-((4-cyclopentylpent-3-en-1-yl)oxy)-3-ethoxybenzaldehyde, 3-ethoxy-4-((4-methyldec-3-en-1-yl)oxy)benzaldehyde, 3-ethoxy-4-((4-methyldodec-3-en-1-yl)oxy)benzaldehyde, 3-ethoxy-4-(tridec-1-en-4-yloxy)benzaldehyde, 4-(4-(tetradeca-1,13-dien-4-yloxy)phenyl)butan-2-one, 3-ethoxy-4-(tetradeca-1,13-dien-4-yloxy)benzaldehyde, 3-methoxy-4-(tetradeca-1,13-dien-4-yloxy)benzaldehyde.

Furthermore, there is provided the use of the compound of formula (I) as precursor selected from the group consisting of (Z)-4-(4-(non-6-en-4-yloxy)phenyl)butan-2-one, 4-(dec-1-en-4-yloxy)-3-ethoxybenzaldehyde, (S,E)-4-(4-(undec-2-en-5-yloxy)phenyl)butan-2-one, (S,E)-4-(4-(dodec-3-en-6-yloxy)phenyl)butan-2-one, (S,E)-2-ethoxy-4-methyl-1-(undec-2-en-5-yloxy)benzene, (S,E)-1-ethoxy-4-methyl-2-(undec-2-en-5-yloxy)benzene, (S,E)-2-(dodec-3-en-6-yloxy)-1-ethoxy-4-methylbenzene, (S,E)-1-(dodec-3-en-6-yloxy)-2-ethoxy-4-methylbenzene, methyl (S,Z)-12-(2-ethoxyphenoxy)octadec-9-enoate, (E)-4-(4-((4-methylnon-3-en-1-yl)oxy)phenyl)butan-2-one, (E)-3-ethoxy-4-((4-methyloct-3-en-1-yl)oxy)benzaldehyde; (E)-4-(4-((4-methyloct-3-en-1-yl)oxy)phenyl)butan-2-one, 4-(4-(((3E,5E)-4-methylhepta-3,5-dien-1-yl)oxy)phenyl)butan-2-one, (E)-4-(4-((4-methylhepta-3,6-dien-1-yl)oxy)phenyl)butan-2-one; (E)-4-allyl-1-((4,8-dimethylnona-3,7-dien-1-yl)oxy)-2-methoxybenzene, (E)-4-((4,8-dimethylnona-3,7-dien-1-yl)oxy)-3-ethoxybenzaldehyde.

Furthermore, there is provided the use of the compound of formula (I) as precursor, wherein the precursor is represented by the compound of formula (III)

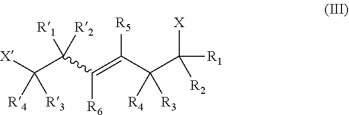

wherein
R₁-R₆ is independently selected from the group consisting of H, Me or linear or branched C2-C20 hydrocarbon groups, bearing up to 4 heteroatoms selected from the group consisting of O, S, and N, and bearing up to three double bonds or two triple bonds, and bearing up to three rings, phenyl, benzyl, substituted aryl or hetero-aryl units, 1- or 2-naphthyl,
X is representing the fragment as defined above for the compound of formula (I),
the wavy bond is indicating an unspecified configuration of the adjacent double bond,
and wherein R′₁, R′₂, R′₃, R′₄, and X′ can be selected from the corresponding groups of R₁, R₂, R₃, R₄, and X, respectively, and the sum of all carbon atoms of R₁-R₆ and R′₁-R′₄ is at least 5 or more.

For example, there is provided the use of the compound of formula (I) as precursor, wherein the precursor is represented by the compound of formula (III)
wherein R₁-R₆ is independently selected from the group consisting of H, Me, C2-C20 branched or linear alkyl or alkenyl or alkynyl, cycloalkyl, cycloalkenyl, phenyl, benzyl, phenylethyl or substituted phenylethyl, substituted aryl or hetero-aryl units, 1- or 2-naphthyl, alkoxycarbonylates (—RO(C═O)—R′), alkylcarboxylates (—R(C═O)—O—R′), ethers, aldehydes, ketones, alcohols,
X is representing the fragment as defined above for the compound of formula (I),
the wavy bond is indicating an unspecified configuration of the adjacent double bond,
and wherein R′₁, R′₂, R′₃, R′₄, and X′ can be selected from the corresponding groups of R₁, R₂, R₃, R₄, and X, respectively, and the sum of all carbon atoms of R₁-R₆ and R′₁-R′₄ is at least 5 or more.

The compound of formula (III) is able to release two phenolic fragrant compounds HX and HX′, which could be same or different. It is further able to release the residue connecting the fragments X, X′.

In a further embodiment, there is provided a compound of formula (I)

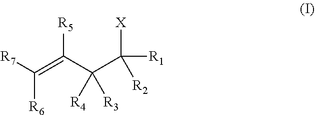

wherein
R₁-R₇ is independently selected from the group consisting of H, Me or linear or branched C2-C20 hydrocarbon groups, bearing up to 4 heteroatoms selected from the group consisting of O, S, and N, and bearing up to three double bonds or two triple bonds, and bearing up to three rings, phenyl, benzyl, substituted aryl or hetero-aryl units, 1- or 2-naphthyl,
or R₆ and R₇ form together a double bond to a carbon atom, which is bearing one or two C1-C10 alkyl or C2-C10 alkenyl substituents, while the other substituents have the same meaning as previously defined;
or R₁ and R₆, R₁ and R₃, R₁ and R₅, R₃ and R₅, R₃ and R₆, R₅ and R₇, R₆ and R₇, R₁ and R₂, or R₃ and R₄, form together with the carbon atoms of the chain they are attached to a 5 or 6 membered ring bearing up to 2 heteroatoms selected from the group consisting of O, S and N, that is optionally substituted with an alkyl chain, while the other substituents have the same meaning as previously defined;
and the sum of all carbon atoms of R₁-R₇ is at least 5 or more;
or R₇ is —CR′₃R′₄—CR′₁R′₂—X′, wherein R′₁, R′₂, R′₃, R′₄, and X′ can be selected from the corresponding groups of R₁, R₂, R₃, R₄, and X, while the other substituents have the same meaning as previously defined, and the sum of all carbon atoms of R₁-R₆ and R′₁-R′₄ is at least 5 or more;
and wherein X is representing the following fragment

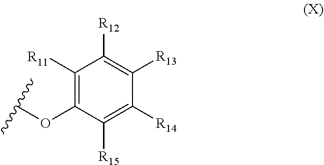

wherein
R₁₁-R₁₅ is independently selected from the group consisting of H, linear or branched C1-C5 alkyl, linear or branched C2-C5 alkenyl, linear or branched C2-C5 alkynyl, OH, C1-C4-alkoxy, CHO, C(O)Me, C(O)Et, hexanoyl, heptanoyl, octanoyl, propanoyl, 3-phenylpropanoyl, 5-methylhexanoyl, (CH₂)₂C(O)CH₃, (4-methyl-3,6-dihydro-2H-pyran-2-yl), CH₂—O-Me, CH₂—O-Et, CH₂—O-iPr, CH₂—OH, Cl, Br, phenyl, CH(OR₁₆)(OR₁₇) wherein R₁₆ and R₁₇ are independently selected from the group consisting of C1-C5 alkyl or R₁₆ and R₁₇ form together with the carbon atoms of the chain they are attached to a 5 or 6 membered ring, C═N—OR₁₈ wherein R₁₈ is C1-C5 alkyl, CO₂R₁₉ wherein R₁₉ is selected from the group consisting of linear or branched C1-C8 alkyl, linear or branched C2-C8 alkenyl, linear or branched C2-C8 alkynyl, cycloalkyl and aryl;
or R₁₁ and R₁₂ or R₁₂ and R₁₃ may form together with the carbon atoms they are attached to C1-C5 alkyl substituted or unsubstituted, saturated or unsaturated 5 and 6 membered rings containing C, O and/or N atoms.

For example, there is provided the compound of formula (I)

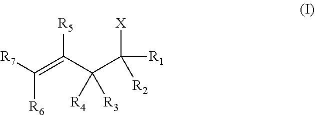

wherein
R₁-R₇ is independently selected from the group consisting of H, Me, C2-C20 branched or linear alkyl or alkenyl or alkynyl, cycloalkyl, cycloalkenyl, phenyl, benzyl, phenylethyl or substituted phenylethyl, substituted aryl or hetero-aryl units, 1- or 2-naphthyl, alkoxycarbonylates (—RO(C═O)—R′), alkylcarboxylates (—R(C═O)—O—R′), ethers, aldehydes, ketones, alcohols;
or R₆ and R₇ form together a double bond to a carbon atom, which is bearing one or two C1-C10 alkyl or C2-C10 alkenyl substituents, while the other substituents have the same meaning as previously defined;
or R₁ and R₆, R₁ and R₃, R₁ and R₅, R₃ and R₅, R₃ and R₆, R₅ and R₇, R₆ and R₇, R₁ and R₂, or R₃ and R₄, form together with the carbon atoms of the chain they are attached to a 5 or 6 membered ring bearing up to 2 heteroatoms selected from the group consisting of O, S and N, that is optionally substituted with an alkyl chain, while the other substituents have the same meaning as previously defined;
and the sum of all carbon atoms of R₁-R₇ is at least 5 or more;
or R₇ is —CR′₃R′₄—CR′₁R′₂—X′, wherein R′₁, R′₂, R′₃, R′₄, and X′ can be selected from the corresponding groups of R₁, R₂, R₃, R₄, and X, while the other substituents have the same meaning as previously defined, and the sum of all carbon atoms of R₁-R₆ and R′₁-R′₄ is at least 5 or more;
and wherein X is representing the following fragment

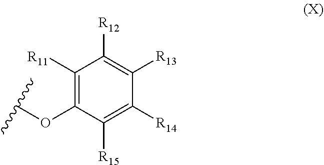

wherein
R₁₁ is selected from the group consisting of H, C1-C5 alkyl, OH, OMe, OEt, O-n-Pr, O-iso-Pr, hexanoyl, heptanoyl, octanoyl, propanoyl, 3-phenylpropanoyl, 5-methylhexanoyl, Cl, Br, phenyl, CO₂R₁₉, wherein R₁₉ is selected from the group consisting of H, Me, Et, 2-methylpropyl, hexan-2-yl, 2-isopropoxyethyl, 3-methylbut-2-en-1-yl, n-pentyl, hexyl, benzyl, cyclohexyl, cis-3-hexenyl, 2-ethylhexyl, 3-methylhex-2-en-1-yl, 2-hept-4-enyl, 1-(3-methyl-2-hexenyl), (CH₂)₂-phenyl, para-tolyl;
R₁₂ is selected from the group consisting of H, OH, C1-C5 alkyl, C2-C5 alkenyl, C1-C5 alkoxy;
R₁₃ is selected from the group consisting of H, branched or linear C1-C5 alkyl, branched or linear C2-C5 alkenyl, CHO, CO₂H, CO₂Me, CO₂Et, C(O)Me, C(O)Et, (CH₂)₂C(O)CH₃; (4-methyl-3,6-dihydro-2H-pyran-2-yl), CH₂—O-Me, CH₂—O-Et, CH₂—O-iPr, CH₂—OH, Cl, propanoyl, CO₂R₁₉, wherein R₁₉ is selected from the group consisting of H, Me, Et, 2-methylpropyl, hexan-2-yl, 2-isopropoxyethyl, 3-methylbut-2-en-1-yl, n-pentyl, hexyl, benzyl, cyclohexyl, cis-3-hexenyl, 2-ethylhexyl, 3-methylhex-2-en-1-yl, 2-hept-4-enyl, 1-(3-methyl-2-hexenyl), (CH₂)₂-phenyl, para-tolyl
R₁₄ is selected from the group consisting of H, OH, C1-C5 alkyl, C2-C5 alkenyl, C1-C5 alkoxy;
R₁₅ is selected from the group consisting of H, C1-C5 alkyl, Br,
or R₁₁ and R₁₂ or R₁₂ and R₁₃ may form together with the carbon atoms they are attached to C1-C5 alkyl substituted or unsubstituted, saturated or unsaturated 5 and 6 membered rings containing C, O and/or N atoms.

For example, the compound of formula (I) can be selected from the group consisting of methyl (S,Z)-12-(2-ethoxy-4-formylphenoxy)octadec-9-enoate, methyl (S,Z)-12-(4-(3-oxobutyl)phenoxy)octadec-9-enoate, methyl (S,Z)-2-hydroxy-4-((18-methoxy-18-oxooctadec-9-en-7-yl)oxy)-3,6-dimethylbenzoate, 3-ethoxy-4-((2-methylundec-2-en-5-yl)oxy)benzaldehyde, 4-(4-((5-methylocta-1,5-dien-4-yl)oxy)phenyl)butan-2-one, 2-methoxy-1-((5-methylocta-1,5-dien-4-yl)oxy)-4-(prop-1-en-1-yl)benzene, methyl 2-hydroxy-3,6-dimethyl-4-((5-methylocta-1,5-dien-4-yl)oxy)benzoate, 4-(4-((1-phenylbut-3-en-1-yl)oxy)phenyl)butan-2-one, 4-(4-((octadeca-9,11-dien-7-yl)oxy)phenyl)butan-2-one, 4,4′-((octadec-9-ene-7,12-diylbis(oxy))bis(4,1-phenylene))bis(butan-2-one), 3-ethoxy-4-((5-methylocta-1,5-dien-4-yl)oxy)benzaldehyde, (Z)-4-(deca-1,7-dien-4-yloxy)-3-ethoxybenzaldehyde, 2-methoxy-1-(((Z)-nona-1,6-dien-4-yl)oxy)-4-(prop-1-en-1-yl)benzene, 4-(4-(((1S,2S,5R)-5-methyl-2-(prop-1-en-2-yl)cyclohexyl)oxy)phenyl)butan-2-one, 3-ethoxy-4-((1-phenylnon-1-en-4-yl)oxy)benzaldehyde, 4-((2,6-dimethylhepta-4,5-dien-2-yl)oxy)-3-ethoxybenzaldehyde, (Z)-3-ethoxy-4-(undec-3-en-6-yloxy)benzaldehyde, 3-ethoxy-4-((2,6,10-trimethylundeca-4,5,9-trien-2-yl)oxy)benzaldehyde, (E)-3-ethoxy-4-(non-6-en-4-yloxy)benzaldehyde, 3-ethoxy-4-(((3E,7E)-4,8,12-trimethyltrideca-3,7,11-trien-1-yl)oxy)benzaldehyde, 3-ethoxy-4-((2-methyl-1-phenylhex-4-en-2-yl)oxy)benzaldehyde, 4-(dec-1-en-4-yloxy)-3-ethoxybenzaldehyde, 4-(4-(dec-1-en-4-yloxy)phenyl)butan-2-one, methyl 4-(dec-1-en-4-yloxy)-2-hydroxy-3,6-dimethylbenzoate, methyl 2-(tetradec-1-en-4-yloxy)benzoate, 1-(2-(tetradec-1-en-4-yloxy)phenyl)propan-1-one, 3-ethoxy-4-(tetradec-1-en-4-yloxy)benzaldehyde, (Z)-3-ethoxy-4-(pentadec-3-en-6-yloxy)benzaldehyde, (E)-3-ethoxy-4-(pentadec-3-en-6-yloxy)benzaldehyde, 3-ethoxy-4-(((3Z,6Z)-nona-3,6-dien-1-yl)oxy)benzaldehyde, 4-(4-(((3Z,6Z)-nona-3,6-dien-1-yl)oxy)phenyl)butan-2-one, 1-methoxy-2-(((3Z,6Z)-nona-3,6-dien-1-yl)oxy)benzene, 2-(((3Z,6Z)-nona-3,6-dien-1-yl)oxy)naphthalene, 3-methoxy-4-(((3Z,6Z)-nona-3,6-dien-1-yl)oxy)benzaldehyde, 3-methoxy-4-(tridec-1-en-4-yloxy)benzaldehyde, 4-(4-(tridec-1-en-4-yloxy)phenyl)butan-2-one, 2-ethoxy-4-(methoxymethyl)-1-(tridec-1-en-4-yloxy)benzene, 3-methoxy-4-(tetradec-1-en-4-yloxy)benzaldehyde, 4-(4-(pentadec-3-en-6-yloxy)phenyl)butan-2-one, ethyl 2-(2-ethoxy-4-formylphenoxy)hept-4-enoate, 3-ethoxy-4-(((3Z,8Z)-undeca-3,8-dien-6-yl)oxy)benzaldehyde, 3-ethoxy-4-(((3Z,8Z)-undeca-3,8-dien-6-yl)oxy)benzaldehyde, 3-ethoxy-4-((4-methylnon-3-en-1-yl)oxy)benzaldehyde, 3-ethoxy-4-(undec-2-en-5-yloxy)benzaldehyde, 4-(dodec-3-en-6-yloxy)-3-ethoxybenzaldehyde, methyl (Z)-12-(2-methoxyphenoxy)octadec-9-enoate, 1-(dec-1-en-4-yloxy)-2-ethoxy-4-methylbenzene, 2-(dec-1-en-4-yloxy)-1-ethoxy-4-methylbenzene, 3-ethoxy-4-((4-methyl-6-phenylhex-3-en-1-yl)oxy)benzaldehyde, (E)-3-ethoxy-4-((4-phenylpent-3-en-1-yl)oxy)benzaldehyde, 3-ethoxy-4-((4-(4-methoxyphenyl)pent-3-en-1-yl)oxy)benzaldehyde, 3-ethoxy-4-((4-methyl-5-phenylpent-3-en-1-yl)oxy)benzaldehyde, (E)-3-ethoxy-4-((4-(naphthalen-2-yl)pent-3-en-1-yl)oxy)benzaldehyde, 4-(4-((4-methyl-6-phenylhex-3-en-1-yl)oxy)phenyl)butan-2-one, 4-(4-((4-(4-methoxyphenyl)pent-3-en-1-yl)oxy)phenyl)butan-2-one, 3-ethoxy-4-((6-(4-methoxyphenyl)-4-methylhex-3-en-1-yl)oxy)benzaldehyde, 4-(4-((6-(4-methoxyphenyl)-4-methylhex-3-en-1-yl)oxy)phenyl)butan-2-one, methyl 2-((4-methyl-6-phenylhex-3-en-1-yl)oxy)benzoate, 1-(2-((4-methyl-6-phenylhex-3-en-1-yl)oxy)phenyl)propan-1-one, 3-ethoxy-4-((4-methyl-7-phenylhept-3-en-1-yl)oxy)benzaldehyde, 4-(4-((4-methyl-7-phenylhept-3-en-1-yl)oxy)phenyl)butan-2-one, (Z)-4-(4-(non-6-en-4-yloxy)phenyl)butan-2-one, 4-(dec-1-en-4-yloxy)-3-ethoxybenzaldehyde, (S,E)-4-(4-(undec-2-en-5-yloxy)phenyl)butan-2-one, (S,E)-4-(4-(dodec-3-en-6-yloxy)phenyl)butan-2-one, (S,E)-2-ethoxy-4-methyl-1-(undec-2-en-5-yloxy)benzene, (S,E)-1-ethoxy-4-methyl-2-(undec-2-en-5-yloxy)benzene, (S,E)-2-(dodec-3-en-6-yloxy)-1-ethoxy-4-methylbenzene, (S,E)-1-(dodec-3-en-6-yloxy)-2-ethoxy-4-methylbenzene, methyl (S,Z)-12-(2-ethoxyphenoxy)octadec-9-enoate, (E)-4-(4-((4-methylnon-3-en-1-yl)oxy)phenyl)butan-2-one, (E)-3-ethoxy-4-((4-methyloct-3-en-1-yl)oxy)benzaldehyde; (E)-4-(4-((4-methyloct-3-en-1-yl)oxy)phenyl)butan-2-one, 4-(4-(((3E,5E)-4-methylhepta-3,5-dien-1-yl)oxy)phenyl)butan-2-one, (E)-4-(4-((4-methylhepta-3,6-dien-1-yl)oxy)phenyl)butan-2-one; (E)-4-allyl-1-((4,8-dimethylnona-3,7-dien-1-yl)oxy)-2-methoxybenzene, (E)-4-((4,8-dimethylnona-3,7-dien-1-yl)oxy)-3-ethoxybenzaldehyde, 4-(((3E,9Z)-dodeca-3,9-dien-1-yl)oxy)-3-ethoxybenzaldehyde, methyl 4-(((3E,9Z)-dodeca-3,9-dien-1-yl)oxy)-2-hydroxy-3,6-dimethylbenzoate, 4-(deca-1,7-dien-4-yloxy)-3-ethoxybenzaldehyde, 4-((6-(benzo[d][1,3]dioxol-5-yl)-4-methylhex-3-en-1-yl)oxy)-3-ethoxybenzaldehyde, 4-(4-((6-(benzo[d][1,3]dioxol-5-yl)-4-methylhex-3-en-1-yl)oxy)phenyl)butan-2-one, 3-ethoxy-4-((4-methyl-6-(2,6,6-trimethylcyclohex-1-en-1-yl)hex-3-en-1-yl)oxy)benzaldehyde, 4-(4-((4-methyl-6-(2,6,6-trimethylcyclohex-1-en-1-yl)hex-3-en-1-yl)oxy)phenyl)butan-2-one, 3-ethoxy-4-((4-methyl-6-(2,6,6-trimethylcyclohex-1-en-1-yl)hexa-3,5-dien-1-yl)oxy)benzaldehyde, 4-((5,9-dimethyldeca-3,8-dien-1-yl)oxy)-3-ethoxybenzaldehyde, 3-ethoxy-4-((6-phenylhex-3-en-1-yl)oxy)benzaldehyde, 3-ethoxy-4-((6-(4-isobutylphenyl)hex-3-en-1-yl)oxy)benzaldehyde, 3-ethoxy-4-(3-(2-(2-(4-methylcyclohex-3-en-1-yl)propyl)cyclopentylidene) propoxy)benzaldehyde, 4-((4-cyclopentylpent-3-en-1-yl)oxy)-3-ethoxybenzaldehyde, 3-ethoxy-4-((4-methyldec-3-en-1-yl)oxy)benzaldehyde, 3-ethoxy-4-((4-methyldodec-3-en-1-yl)oxy)benzaldehyde, 3-ethoxy-4-(tridec-1-en-4-yloxy)benzaldehyde, 4-(4-(tetradeca-1,13-dien-4-yloxy)phenyl)butan-2-one, 3-ethoxy-4-(tetradeca-1,13-dien-4-yloxy)benzaldehyde, 3-methoxy-4-(tetradeca-1,13-dien-4-yloxy)benzaldehyde.

The compounds of formula (I) described above release phenolic fragrant compounds HX of formula (II) upon exposure to ambient air by oxidation in the presence or absence of light over a long period of time, e.g. several days such as 2-7 days or even longer.

In a further aspect of the present invention, there is provided a method to release a phenolic fragrant compound of formula (II), wherein a compound of formula (I) is exposed to ambient air in the presence or absence of light.

For example, the phenolic fragrant compound HX of formula (II) that can be released from the compound of formula (I) upon exposure to ambient air in the presence or absence of light can be selected from the group consisting of Vanillin (4-hydroxy-3-methoxybenzaldehyde), Ethyl vanillin (3-ethoxy-4-hydroxybenzaldehyde), Eugenol (4-allyl-2-methoxyphenol), Isoeugenol ((E)-2-methoxy-4-(prop-1-en-1-yl)phenol), Raspberry ketone (4-(4-hydroxyphenyl)butan-2-one), Evernyl (methyl 2,4-dihydroxy-3,6-dimethylbenzoate), Seamoss (methyl 2,4-dihydroxy-3-methylbenzoate), Cresol (2-methylphenol, 3-methylphenol, or 4-methylphenol), Ultravanil (2-ethoxy-4-methylphenol), Guaiacol (2-Methoxyphenol), Vinyl Guaiacol (4-Ethenyl-2-methoxyphenol), Methyl Diantilis (2-Ethoxy-4-(methoxymethyl)phenol), Thymol (5-Methyl-2-(propan-2-yl)phenol), Carvacrol (5-isopropyl-2-methylphenol), Eugewhite (2-methoxy-4-(4-methyl-3,6-dihydro-2H-pyran-2-yl)phenol), Zingerone (4-(4-hydroxy-3-methoxyphenyl)butan-2-one), Karmaflor ((4Z)-hept-4-en-2-yl 2-hydroxybenzoate), Saffiano (3-methylbenzofuran-5-ol), Carvacrol (5-isopropyl-2-methylphenol), Creosol (2-methoxy-4-methylphenol), Orcinyl 3 (3-methoxy-5-methylphenol), Hexyl salicylate (hexyl 2-hydroxybenzoate), Cyclohexyl salicylate (cyclohexyl 2-hydroxybenzoate), Phenyl ethyl salicylate (phenethyl 2-hydroxybenzoate), Octyl salicylate (2-ethylhexyl 2-hydroxybenzoate), Yasminate (3-methylhex-2-en-1-yl 2-hydroxybenzoate), Ethyl salicylate (ethyl 2-hydroxybenzoate), Hexenyl-3-cis salicylate ((Z)-hex-3-en-1-yl 2-hydroxybenzoate), Isobutyl salicylate (isobutyl 2-hydroxybenzoate), Methyl salicylate (methyl 2-hydroxybenzoate), Vanitrop (2-ethoxy-5-(prop-1-en-1-yl)phenol), Methylpentyl salicylate (hexan-2-yl 2-hydroxybenzoate), Para-cresyl salicylate (p-tolyl 2-hydroxybenzoate), Prenyl salicylate (3-methylbut-2-en-1-yl 2-hydroxybenzoate), Amyl salicylate (pentyl 2-hydroxybenzoate), Benzyl salicylate (benzyl 2-hydroxybenzoate), Sakura salicylate (2-isopropoxyethyl 2-hydroxybenzoate), 3-propylphenol, 4-ethylphenol and 2-ethoxyphenol, and 1-(2-hydroxyphenyl)-3-phenylpropan-1-one.

For example, the phenolic fragrant compound HX of formula (II) that can be released from the compound of formula (I) upon on exposure to ambient air in the presence or absence of light is a phenol having at least one substituent at the aromatic ring, the at least one substituent having 1 carbon atom or more, for example 2 to 4 carbon atoms or more.

The compounds of formula (I) release phenolic fragrant compounds HX of formula (II) upon exposure to ambient air in the presence or absence of light over a long period of time (e.g. several days such as 2-7 days or even longer).

Exposure of the precursor compound to ambient air means exposure to molecular oxygen which might be responsible for the oxidative cleavage of the compound of formula (I) and the release of the compound of formula (II). The concentration of oxygen in the air is sufficient for cleaving the compound of formula (I) so that the cleavage products can be detected in the ambient air, e.g. by olfaction or GC-MS analysis of collected organic volatiles from the precursor headspace.

The compounds of formula (I) are very stable when their exposure to ambient air is limited or prevented, i.e. when stored in neat form in appropriate containers protected from air and light, or when stored in proper solvents, for example in ethanol, isopropanol, diethylene glycol monoethyl ether, glycerol, propylene glycol, 1,2-butylene glycol, dipropylene glycol, isopropyl myristate, triethyl citrate, diethyl phthalate, triacetine and/or diacetine, or when incorporated in consumer products such as detergent, shampoo and fabric conditioner. Thus the compounds of formula (I) may find use in a broad range of consumer products in which a prolonged and defined release of fragrant compounds is desired.

In some embodiments, stabilizing compounds, for example alpha tocopherol, EDTA, ascorbic acid, BHT, Tinoguard TT, can be added to the compounds of formula (I), for example in 0.01-1% by weight, to limit or prevent premature cleavage of the compound of formula (I) and the release of the compound of formula (II). In particular, the stabilizing compounds can be used to enhance the stability of the neat compounds of formula (I).

The compound of formula (I) may be used alone, or in combination with known odorant molecules selected from the extensive range of natural products, and synthetic molecules currently available, such as essential oils, alcohols, aldehydes and ketones, ethers and acetals, esters and lactones, macrocycles and heterocycles, and/or in admixture with one or more ingredients or excipients conventionally used in conjunction with odorants in perfume compositions, for example, carrier materials, and other auxiliary agents commonly used in the art.

For example, the compound of formula (I) may be used in combination with the free phenolic fragrant compound HX. Such a combination ensures a continuous perception of the phenolic fragrant compound HX with time. It is particularly useful, if the discoloration effect of free phenolic fragrant compound HX is not prominent or is not relevant for the consumer.

In a further aspect, the compound of formula (I) may be used in combination with other fragrance precursors, either with further compounds of formula (I) or with precursors possessing a different chemical structure. A combination of precursors allows releasing a perfume accord.

In a further aspect, there is provided a fragrance composition comprising at least one compound of formula (I). For example, the fragrance composition is further comprising one or more known odorant molecules, and/or one or more ingredients or excipients conventionally used in conjunction with odorants in perfume compositions

As used herein, “carrier material” means a material which is practically neutral from a odourant point of view, i.e. a material that does not significantly alter the organoleptic properties of odorants.

The term “auxiliary agent” refers to ingredients that might be employed in a fragrance composition for reasons not specifically related to the olfactive performance of said composition. For example, an auxiliary agent may be an ingredient that acts as an aid to processing a fragrance ingredient or ingredients, or a composition containing said ingredient(s), or it may improve handling or storage of a fragrance ingredient or composition containing same. It might also be an ingredient that provides additional benefits such as imparting color or texture. It might also be an ingredient that imparts light resistance or chemical stability to one or more ingredients contained in a perfume composition. A detailed description of the nature and type of adjuvants commonly used in perfume compositions containing same cannot be exhaustive, but it has to be mentioned that said ingredients are well known to a person skilled in the art.

As used herein, “fragrance composition” means any composition comprising the compound of formula (I) and a base material, e.g. a diluent conventionally used in conjunction with odourants, such as diethyl phthalate (DEP), dipropylene glycol (DPG), isopropyl myristate (IPM), pentane-1,2-diol, triethyl citrate (TEC) and alcohol (e.g. ethanol). Optionally, the composition may comprise an anti-oxidant adjuvant. Said anti-oxidant may be selected from Tinogard® TT (BASF), Tinogard® Q (BASF), Tocopherol (including its isomers, CAS 59-02-9; 364-49-8; 18920-62-2; 121854-78-2), 2,6-bis(1,1-dimethylethyl)-4-methylphenol (BHT, CAS 128-37-0) and related phenols, hydroquinones (CAS 121-31-9).

The following non limiting list comprises examples of known odourant molecules, which may be combined with the compound of formula (I) in a fragrance composition:
Essential oils and extracts, e.g. castoreum, costus root oil, oak moss absolute, geranium oil, tree moss absolute, basil oil, fruit oils, such as bergamot oil and mandarine oil, myrtle oil, palmarose oil, patchouli oil, petitgrain oil, jasmine oil, rose oil, sandalwood oil, wormwood oil, lavender oil and/or ylang-ylang oil;
Alcohols, e.g. cinnamic alcohol ((E)-3-phenylprop-2-en-1-ol); cis-3-hexenol ((Z)-hex-3-en-1-ol); Citronellol (3,7-dimethyloct-6-en-1-ol); dihydro myrcenol (2,6-dimethyloct-7-en-2-ol); Ebanol™ ((E)-3-methyl-5-(2,2,3-trimethylcyclopent-3-en-1-yl)pent-4-en-2-ol); eugenol (4-allyl-2-methoxyphenol); ethyl linalool ((E)-3,7-dimethylnona-1,6-dien-3-ol); farnesol ((2E,6Z)-3,7,11-trimethyldodeca-2,6,10-trien-1-ol); geraniol ((E)-3,7-dimethylocta-2,6-dien-1-ol); Super Muguet™ ((E)-6-ethyl-3-methyloct-6-en-1-ol); linalool (3,7-dimethylocta-1,6-dien-3-ol); menthol (2-isopropyl-5-methylcyclohexanol); Nerol (3,7-dimethyl-2,6-octadien-1-ol); phenyl ethyl alcohol (2-phenylethanol); Rhodinol™ (3,7-dimethyloct-6-en-1-ol); Sandalore™ (3-methyl-5-(2,2,3-trimethylcyclopent-3-en-1-yl)pentan-2-ol); terpineol (2-(4-methylcyclohex-3-en-1-yl)propan-2-ol); or Timberol™ (1-(2,2,6-trimethylcyclohexyl)hexan-3-ol); 2,4,7-trimethylocta-2,6-dien-1-ol, and/or [1-methyl-2(5-methylhex-4-en-2-yl)cyclopropyl]-methanol;
Aldehydes and ketones, e.g. anisaldehyde (4-methoxybenzaldehyde); alpha amyl cinnamic aldehyde (2-benzylideneheptanal); Georgywood™ (1-(1,2,8,8-tetramethyl-1,2,3,4,5,6,7,8-octahydronaphthalen-2-yl)ethanone); Hydroxycitronellal (7-hydroxy-3,7-dimethyloctanal); Iso E Super® (1-(2,3,8,8-tetramethyl-1,2,3,4,5,6,7,8-octahydronaphthalen-2-yl)ethanone); Isoraldeine® ((E)-3-methyl-4-(2,6,6-trimethylcyclohex-2-en-1-yl)but-3-en-2-one); Hedione® (methyl 3-oxo-2-pentylcyclopentaneacetate); Nympheal (3-(4-isobutyl-2-methylphenyl)propanal); Mahonial (5,9-dimethyl-9-hydroxy-decen-4-al); maltol; methyl cedryl ketone; methylionone; verbenone; and/or vanillin;
Ether and acetals, e.g. Ambrox® (3a,6,6,9a-tetramethyl-2,4,5,5a,7,8,9,9b-octahydro-1H-benzo[e][1]benzofuran); geranyl methyl ether ((2E)-1-methoxy-3,7-dimethylocta-2,6-diene); rose oxide (4-methyl-2-(2-methylprop-1-en-1-yl)tetrahydro-2H-pyran); and/or Spirambrene® (2′,2′,3,7,7-pentamethylspiro[bicyclo[4.1.0]heptane-2,5′-[1,3]dioxane]);
Esters and lactones, e.g. benzyl acetate; cedryl acetate ((1 S,6R,8aR)-1,4,4,6-tetramethyloctahydro-1H-5,8a-methanoazulen-6-yl acetate); γ-decalactone (6-pentyltetrahydro-2H-pyran-2-one); Helvetolide® (2-(1-(3,3-dimethylcyclohexyl)ethoxy)-2-methylpropyl propionate); γ-undecalactone (5-heptyloxolan-2-one); and/or vetiveryl acetate ((4,8-dimethyl-2-propan-2-ylidene-3,3a,4,5,6,8a-hexahydro-1H-azulen-6-yl) acetate
Macrocycles, e.g. Ambrettolide ((Z)-oxacycloheptadec-10-en-2-one); ethylene brassylate (1,4-dioxacycloheptadecane-5,17-dione); and/or Exaltolide® (16-oxacyclohexadecan-1-one); and
Heterocycles, e.g. isobutylquinoline (2-isobutylquinoline).

Overall, the compounds of formula (I) can be used alone, as a mixture thereof, or in combination with other fragrance ingredients and/or precursors thereof. Such other fragrance ingredients are also described, for example, in “Perfume and Flavor Chemicals”, S. Arctander, Ed., Vol. I & II, Allured Publishing Corporation, Carol Stream, USA, 2003 and include fragrance compounds of natural or synthetic origin and essential oils.

In a further aspect, there is provided a consumer product comprising at least one compound of formula (I) and a consumer product base.

For example, the consumer product according to the invention is selected from the group consisting of detergents and cleaning agents, hygiene or care products, preferably in the field of body and hair care, cosmetics and household, preferably from the group consisting of perfume extracts, eau de parfums, eau de toilettes, aftershave lotions, eau de colognes, pre-shave products, splash colognes, perfumed refreshing wipes, acidic, alkaline or neutral detergents, textile fresheners, ironing aids, liquid detergents, powder detergents, laundry pre-treatments, fabric softeners, laundry sheets, washing soaps, washing tablets, dish bar soaps, disinfectants, surface disinfectants, air fresheners, aerosol sprays, waxes and polishes, body care products, hand creams and lotions, foot creams and lotions, depilatory creams and lotions, after-shave creams and lotions, tanning creams and lotions, hair care products (in liquid or solid form), dry shampoo, deodorants, antiperspirants, decorative cosmetic products, candles, lamp oils, incense sticks, insecticides, repellents and fuels.

The consumer product for example is selected from fine fragrance, personal care products (body care products, hair care products, cosmetic products) fabric care products, home care products and air care products. As used herein, “consumer product base” means a composition for use as a consumer product to fulfill specific actions, such as cleaning, softening, and caring or the like.

Personal care products to which the compound of formula (I) can be added include for example all kinds of body care products. Especially interesting products are hair care products, for example shampoos, conditioners and hairsprays, and skin care products, like lotions or creams. Furthermore, the compound of formula (I) may be added to soaps, bath and shower gels and deodorants. The compound of formula (I) can be added to cosmetic products.

Home care products to which the compound of formula (I) can be added include all kinds of detergents, window cleaners, hard surface cleaners, all-purpose cleaners and furniture polishes. Preferably, the products are liquids, e.g. fabric detergent or conditioner compositions.

For example, the fabric detergent or conditioner compositions can be employed on different type of fibres, for example on cotton, wool, elastan, lycra, nylon, polyester, or on mixtures thereof. For example, the compounds of formula (I) can act as fragrance precursors in consumer products which further comprise enzymes.

The compound according to formula (I) may be used in a broad range of perfumed consumer products, e.g. in any field of fine and functional perfumery, such as perfumes, air care products, household products, laundry products, body care products and cosmetics. The compound can be employed in widely varying amounts, depending upon the specific article and on the nature and quantity of other odourant ingredients. The proportion of the formula (I) is typically from 0.0001 to 5 weight % of the article. In one embodiment, the compound of formula (I) may be employed in a fabric softener in an amount from 0.001 to 0.3 weight % (e.g. 0.01 to 0.1 including 0.05 weight %). In another embodiment, the compound of formula (I) may be used in fine perfumery but also in consumer products like shampoo, fabric softener or fabric detergents, in amounts from 0.001 to 30 weight % (e.g. up to about 10 or up to 20 weight %), more preferably between 0.01 and 5 weight %. However, these values are given only by way of example, since the experienced perfumer may also achieve effects or may create novel accords with lower or higher concentrations.

In one embodiment there is provided a consumer product comprising an acceptable amount of the compound of formula (I). For example, the fragranced article may comprise 0.000001 weight % to 90 weight % (including 0.00001 weight %; 0.0001 weight %, 0.001 weight %, 0.01 weight %, 0.05 weight %, 0.1 weight %, 0.5 weight %, 1 weight %, 5 weight %, 8 weight %, 10 weight %, 15 weight %, 20 weight %, 25 weight %, 30 weight %, 50 weight %, 60 weight %, 65 weight %) of the compound of formula (I) based on the total amount of the article.

The compound of formula (I) may be employed in a consumer product base simply by directly mixing the compound of the present invention, or a fragrance composition comprising the compound of formula (I), with the consumer product base, or it may, in an earlier step, be entrapped with an entrapment material, for example, polymers, capsules, microcapsules and nanocapsules, liposomes, film formers, absorbents such as carbon or zeolites, cyclic oligosaccharides and mixtures thereof, and then mixed with the consumer product base. The consumer base might further contain entrapment material able to release other fragrant compounds.

Thus, the invention additionally provides a method of manufacturing a consumer product, comprising the incorporation of a compound of formula (I) either by directly admixing it to the consumer product base or by admixing a fragrance composition comprising the compound of formula (I), which may then be mixed with a consumer product base, using conventional techniques and methods. Through the addition of an acceptable amount of the compound of the present invention as hereinabove described, the odour notes of an applied consumer product will be improved, conferred, enhanced, or modified.

Thus, the invention furthermore provides in another aspect a method to confer, enhance, improve or modify the hedonic properties of a fragrance composition or a consumer product, which method comprises adding to said composition or consumer product at least one compound of formula (I).

The compounds of formula (I) may be prepared starting from the respective phenolic fragrant compound HX which is a compound of formula (II) and an unsaturated alkylhalide, for example an alkenyl chloride, bromide or iodide.

Alternatively, the phenolic fragrant compound HX of formula (II) can be converted to a compound of formula (I) via a Mitsunobu reaction with an unsaturated alcohol in the presence of triphenylphosphine (PPh₃) and diisopropyl azodicarboxylate (DIAD)

Therefore, in a further aspect of the present invention, there is provided a method of making a compound of formula (I), comprising the steps of:
a) providing a phenolic fragrant compound HX,
b) reacting it with an unsaturated alkylhalide or an unsaturated alcohol.

In one embodiment, the starting materials for the process to prepare the compound of formula (I) is obtained from renewable resources. With such a starting material also the final product is accessible from renewable resources.

In a further aspect, the compounds of the present invention are biodegradable, as demonstrated by the Manometric Respirometry test (OECD guideline for the testing of materials No. 301F, Paris 1992).

The invention is now further described with reference to the following non-limiting examples. These examples are for the purpose of illustration only and it is understood that variations and modifications can be made by one skilled in the art.

### DESCRIPTION OF FIGURES

FIG. 1 shows the comparison of color stability of a precursor according to the invention and a free phenolic fragrant compound HX when used in a liquid detergent.

FIG. 2 shows the setup of a headspace vial used for analysis of volatiles.

### EXAMPLES

### General:

All reactions were performed under argon using solvents and reagents from commercial suppliers without further purification. Solvents for extraction and chromatography were technical grade and used without further purification. Flash chromatography was performed using commercially available prepacked silica gel cartridges. Unless otherwise noted, a mixture of Heptane:MTBE was used as eluent. NMR spectra were recorded with Bruker AW 400 MHz or Avance III HD 400 MHz instruments. The chemical shifts for ¹H NMR spectra was reported in δ (ppm) referenced to the residual proton signal of the deuterated solvent; coupling constants were expressed in Hertz (Hz). ¹³C NMR spectra were referenced to the carbon signals of the deuterated solvent. The following abbreviations are used: s=singlet, d=doublet, t=triplet, q=quartet, quint.=quintuplet, m=multiplet, dd=double doublet, bs=broad singlet. GC/MS spectral data were obtained from an Agilent 6890 N and MSD 5975 using a column HP-5 MS, 30 m, 0.25 mm, 0.25 μm.

### Example 1: methyl (S,Z)-12-(2-ethoxy-4-formylphenoxy)octadec-9-enoate

To a solution of ethyl vanillin (10.0 g, 60.2 mmol, 1.0 equiv), methyl ricinoleate (22.6 g, 72.2 mmol, 1.2 equiv) and triphenylphosphine (20.5 g, 78.0 mmol) in THF (200 mL) was added DIAD (15.8 g, 78.0 mmol) slowly at r.t. The temperature was kept below 25° C. and the reaction mixture was stirred at r.t. for 16 hours under argon atmosphere. The reaction conversion was monitored by TLC and GC. The mixture was filtered, and the filtrate was condensed by rotary evaporation. The residue was purified by silica gel column chromatography (PE:MTBE=94:6) to give methyl (S,Z)-12-(2-ethoxy-4-formylphenoxy)octadec-9-enoate (33.9 mmol, 15.6 g, yield: 56%) as colorless liquid.

¹H NMR (400 MHz, CDCl₃) δ 9.73 (s, 1H), 7.55-7.16 (m, 2H), 6.89 (d, J=8.3 Hz, 1H), 5.56-5.14 (m, 2H), 4.51-3.88 (m, 3H), 3.56 (s, 3H), 2.46-2.28 (m, 2H), 2.20 (t, J=7.5 Hz, 2H), 2.00-1.88 (m, 2H), 1.72-1.45 (m, 4H), 1.45-1.05 (m, 19H), 0.77 (t, J=6.3 Hz, 3H) ppm. ¹³C NMR (101 MHz, CDCl₃) δ 190.8, 174.1, 154.1, 150.0, 132.6, 130.0, 126.2, 124.2, 114.3, 111.6, 79.5, 64.5, 51.3, 34.0, 33.7, 31.7, 31.6, 29.4, 29.2, 29.1, 29.1, 29.1, 27.4, 25.3, 24.9, 22.5, 14.7, 14.0 ppm. GC/MS (EI): m/z (%): 460 (1) [M⁺], 295 (10), 263 (18), 245 (9), 166 (100), 138 (37), 109 (14), 95 (17), 81 (26).

Odour description (1% solution in EtOH on paper blotter, 24 h): powdery, vanillin, green, fatty.

### Example 2: methyl (S,Z)-12-(4-(3-oxobutyl)phenoxy)octadec-9-enoate

The compound was obtained from 4-(4-hydroxyphenyl)butan-2-one (5.0 g, 30.4 mmol, 1.0 equiv) and methyl (Z)-12-hydroxyoctadec-9-enoate (11.4 g, 36.5 mmol, 1.2 equiv) according to the procedure of example 1 as colorless liquid (16.6 mmol, 7.6 g, 54% yield).

¹H NMR (400 MHz, CDCl₃) δ 7.06 (d, J=7.9 Hz, 2H), 6.80 (d, J=7.9 Hz, 2H), 5.55-5.32 (m, 2H), 4.29-4.06 (m, 1H), 3.65 (s, 3H), 2.89-2.64 (m, 4H), 2.45-2.22 (m, 4H), 2.15-1.89 (m, 5H), 1.73-1.54 (m, 4H), 1.51-1.14 (m, 16H), 0.86 (s, 3H) ppm. ¹³C NMR (101 MHz, CDCl₃) δ 208.0, 174.2, 156.8, 132.9, 132.3, 129.2, 124.7, 116.0, 77.8, 51.4, 45.4, 34.0, 33.8, 31.8, 31.5, 30.0, 29.5, 29.3, 29.2, 29.1, 28.9, 27.4, 25.4, 24.9, 22.6, 14.1 ppm. GC/MS (EI): m/z (%): 458 (3) [M⁺], 294 (7), 261 (19), 203 (19), 164 (100), 107 (63), 55 (30).

Odour description (1% solution in EtOH on paper blotter, 24 h): powdery fruity raspberry, green, fatty

### Example 3: methyl (S,Z)-2-hydroxy-4-((18-methoxy-18-oxooctadec-9-en-7-yl)oxy)-3,6-dimethylbenzoate

The compound was obtained from methyl 2,4-dihydroxy-3,6-dimethylbenzoate (5.0 g, 25.5 mmol, 1.0 equiv) and methyl (Z)-12-hydroxyoctadec-9-enoate (9.6 g, 30.6 mmol, 1.2 equiv) according to the procedure of example 1 as colorless liquid (16.7 mmol, 8.2 g, 66% yield).

¹H NMR (400 MHz, CDCl₃) δ 11.79 (s, 1H), 6.17 (s, 1H), 5.54-5.13 (m, 2H), 4.34-4.11 (m, 1H), 3.81 (s, 3H), 3.57 (s, 3H), 2.41 (s, 3H), 2.38-2.25 (m, 2H), 2.20 (t, J=7.5 Hz, 2H), 1.98 (s, 3H), 1.96-1.89 (m, 2H), 1.67-1.42 (m, 4H), 1.40-1.13 (m, 16H), 0.78 (t, J=6.7 Hz, 3H) ppm. ¹³C NMR (101 MHz, CDCl₃) δ 173.2, 171.5, 161.5, 159.4, 138.7, 131.5, 123.3, 110.8, 106.8, 103.9, 76.5, 50.6, 50.4, 33.0, 32.7, 30.8, 30.7, 28.5, 28.3, 28.2, 28.1, 28.1, 26.4, 24.3, 23.9, 23.7, 21.6, 13.0, 7.0 ppm.

Odour description (1% solution in EtOH on paper blotter, 24 h): powdery mossy, evernyl, green, fatty

### Example 4: 3-ethoxy-4-((2-methylundec-2-en-5-yl)oxy)benzaldehyde

The compound was obtained from 3-ethoxy-4-hydroxybenzaldehyde (3.0 g, 18.1 mmol, 1.0 equiv) and 2-methylundec-2-en-5-ol (3.7 g, 19.9 mmol, 1.1 equiv) according to the procedure of example 1 as colorless liquid (7.2 mmol, 2.4 g, 41% yield).

¹H NMR (400 MHz, CDCl₃) δ 9.83 (s, 1H), 7.49-7.33 (m, 2H), 7.04-6.85 (m, 1H), 5.18 (t, J=7.0 Hz, 1H), 4.45-4.25 (m, 1H), 4.13 (q, J=7.0 Hz, 2H), 2.57-2.21 (m, 2H), 1.81-1.59 (m, 8H), 1.50-1.20 (m, 11H), 0.93-0.76 (m, 3H) ppm. ¹³C NMR (101 MHz, CDCl₃) δ 191.0, 154.4, 150.1, 134.3, 129.9, 126.3, 119.4, 114.4, 111.8, 80.0, 64.6, 33.7, 32.6, 31.8, 29.3, 25.9, 25.4, 22.6, 18.0, 14.7, 14.1 ppm. GC/MS (EI): m/z (%): 332 (3) [M⁺], 263 (1), 167 (42), 137 (21), 111 (25), 69 (100), 55 (30).

Odour description (1% solution in EtOH on paper blotter, 24 h): vanilla (powdery, creamy) dry (dough)

### Example 5: 4-(4-((5-methylocta-1,5-dien-4-yl)oxy)phenyl)butan-2-one

The compound was obtained from 4-(4-hydroxyphenyl)butan-2-one (5.2 g, 31.4 mmol, 1.1 equiv) and 5-methylocta-1,5-dien-4-ol (4.0 g, 28.5 mmol, 1.0 equiv) according to the procedure of example 1 as colorless liquid (17.1 mmol, 4.9 g, 60% yield).

¹H NMR (400 MHz, CDCl₃, mixture of E/Z isomers) δ 7.03 (d, J=8.0 Hz, 2H), 6.80 (d, J=8.0 Hz, 2H), 5.94-5.64 (m, 1H), 5.51-5.38 (m, 1H), 5.18-4.85 (m, 2H), 4.54-4.27 (m, 1H), 2.86-2.32 (m, 6H), 2.12 (s, 3H), 2.07-1.62 (m, 2H), 1.61-1.54 (m, 3H), 0.97-0.83 (m, 3H) ppm. ¹³C NMR (101 MHz, CDCl₃, mixture of E/Z isomers) δ 208.3, 156.9, 156.8, 136.5, 135.5, 134.8, 132.9, 132.9, 132.8, 130.4, 128.9, 128.9, 125.1, 116.7, 116.3, 116.2, 114.6, 85.1, 83.4, 45.5, 45.5, 38.8, 31.7, 30.1, 28.9, 27.1, 20.8, 13.9, 11.1, 11.1, 10.3 ppm. GC/MS (EI): m/z (%): 286 (1) [M⁺], 245 (2), 187 (11), 164 (21), 122 (67), 107 (71), 93 (75), 81 (100). Odour description (1% solution in EtOH on paper blotter, 24 h): fruity (powdery, sweet, raspberry)

### Example 6: 2-methoxy-1-((5-methylocta-1,5-dien-4-yl)oxy)-4-(prop-1-en-1-yl)benzene

The compound was obtained from 2-methoxy-4-(prop-1-en-1-yl)phenol (5.2 g, 31.4 mmol, 1.1 equiv) and 5-methylocta-1,5-dien-4-ol (4.0 g, 28.5 mmol, 1.0 equiv) according to the procedure of example 1 as colorless liquid (13.6 mmol, 3.9 g, 47% yield).

¹H NMR (400 MHz, CDCl₃, mixture of E/Z isomers) δ 6.86 (s, 1H), 6.83-6.70 (m, 2H), 6.31 (d, J=15.7 Hz, 1H), 6.15-6.00 (m, 1H), 5.93-5.64 (m, 1H), 5.49-5.31 (m, 1H), 5.18-4.85 (m, 2H), 4.53-4.32 (m, 1H), 3.85 (s, 3H), 2.81-2.38 (m, 2H), 2.05-1.66 (m, 5H), 1.59 (s, 3H), 0.98-0.83 (m, 3H) ppm. ¹³C NMR (101 MHz, CDCl₃, mixture of E/Z isomers) δ 150.4, 150.3, 147.0, 146.8, 136.5, 135.5, 134.9, 132.8, 131.6, 131.4, 130.9, 130.7, 125.4, 123.8, 123.7, 118.5, 116.9, 116.6, 114.6, 109.5, 109.4, 86.8, 85.1, 56.0, 56.0, 38.5, 31.7, 26.8, 20.8, 18.4, 13.9, 11.0, 10.4 ppm. GC/MS (EI): m/z (%): 286 (1) [M⁺], 245 (1), 166 (100), 149 (9), 81 (19), 55 (6).

Odour description (1% solution in EtOH on paper blotter, 24 h): woody (smokey, cedarwood, pyrogravure)

### Example 7: methyl 2-hydroxy-3,6-dimethyl-4-((5-methylocta-1,5-dien-4-yl)oxy)benzoate

The compound was obtained from methyl 2,4-dihydroxy-3,6-dimethylbenzoate (6.2 g, 31.4 mmol, 1.1 equiv) and 5-methylocta-1,5-dien-4-ol (4.0 g, 28.5 mmol, 1.0 equiv) according to the procedure of example 1 as colorless liquid (9.7 mmol, 3.1 g, 34% yield).

¹H NMR (400 MHz, CDCl₃) δ 11.81 (s, 1H), 6.25 (s, 1H), 5.89-5.69 (m, 1H), 5.52-5.39 (m, 1H), 5.19-4.88 (m, 2H), 4.64-4.43 (m, 1H), 3.90 (s, 3H), 2.86-2.38 (m, 5H), 2.09 (s, 3H), 2.07-1.66 (m, 2H), 1.57 (s, 3H), 1.01-0.87 (m, 3H) ppm. ¹³C NMR (101 MHz, CDCl₃) δ 172.6, 162.2, 160.4, 160.2, 139.4, 139.4, 136.3, 135.2, 134.4, 132.6, 130.5, 125.1, 117.1, 114.7, 111.6, 111.5, 108.9, 108.8, 105.1, 105.0, 84.6, 83.0, 51.7, 38.8, 31.6, 27.1, 24.6, 20.8, 13.9, 11.1, 11.1, 10.2, 8.0 ppm. GC/MS (EI): m/z (%): 318 (4) [M⁺], 277 (4), 245 (8), 196 (31), 164 (76), 122 (43), 81 (100), 67 (28).

Odour description (1% solution in EtOH on paper blotter, 24 h): powdery (evernyl)

### Example 8: 4-(4-((1-phenylbut-3-en-1-yl)oxy)phenyl)butan-2-one

The compound was obtained from 4-(4-hydroxyphenyl)butan-2-one (4.9 g, 29.7 mmol, 1.1 equiv) and 1-phenylbut-3-en-1-ol (4.0 g, 27.0 mmol, 1.0 equiv) according to the procedure of example 1 as colorless liquid (6.8 mmol, 2.0 g, 25% yield).

¹H NMR (400 MHz, CDCl₃) δ 7.38-7.23 (m, 5H), 6.98 (d, J=8.3 Hz, 2H), 6.75 (d, J=8.3 Hz, 2H), 5.98-5.67 (m, 1H), 5.18-4.96 (m, 3H), 2.82-2.50 (m, 6H), 2.09 (s, 3H) ppm. ¹³C NMR (101 MHz, CDCl₃) δ 208.2, 156.5, 141.5, 134.2, 133.1, 129.1, 128.5, 127.6, 126.1, 117.5, 116.0, 80.0, 45.4, 42.9, 30.1, 28.9 ppm. GC/MS (EI): m/z (%): 294 (3) [M⁺], 253 (6), 164 (58), 131 (100), 107 (73), 91 (73), 77 (51), 65 (20).

Odour description (1% solution in EtOH on paper blotter, 24 h): fruity (powdery, sweet, raspberry)

### Example 9: 4-(4-((octadeca-9,11-dien-7-yl)oxy)phenyl)butan-2-one

The compound was obtained from 4-(4-hydroxyphenyl)butan-2-one (1.3 g, 7.9 mmol, 1.5 equiv) and octadec-9-ene-7,12-diol (1.5 g, 5.3 mmol, 1.0 equiv) according to the procedure of example 1 as white solid (0.5 mmol, 0.2 g, 7% yield, by product, the main product is example 10).

¹H NMR (400 MHz, CDCl₃) δ 7.00 (d, J=8.3 Hz, 2H), 6.74 (d, J=8.3 Hz, 2H), 6.36-5.16 (m, 4H), 4.21-3.83 (m, 1H), 2.82-2.70 (m, 2H), 2.68-2.57 (m, 2H), 2.51-2.24 (m, 2H), 2.10-2.01 (m, 4H), 1.62-1.52 (m, 2H), 1.38-1.10 (m, 17H), 0.85-0.74 (m, 6H) ppm. ¹³C NMR (101 MHz, CDCl₃) δ 207.2, 155.8, 134.8, 132.0, 130.0, 128.2, 128.1, 127.3, 115.2, 115.1, 76.8, 44.4, 36.2, 32.8, 30.8, 30.7, 29.1, 28.6, 28.3, 27.9, 27.9, 26.7, 24.4, 21.6, 21.6, 13.1, 13.0 ppm. GC/MS (EI): m/z (%): 412 (8) [M⁺], 261 (42), 203 (58), 179 (1), 164 (22), 107 (85), 91 (24), 81 (50), 67 (100), 55 (84).

Odour description (1% solution in EtOH on paper blotter, 24 h): powdery fruity raspberry

### Example 10: 4,4′-((octadec-9-ene-7,12-diylbis(oxy))bis(4,1-phenylene))bis(butan-2-one)

The compound was obtained from 4-(4-hydroxyphenyl)butan-2-one (1.3 g, 7.9 mmol, 1.5 equiv) and octadec-9-ene-7,12-diol (1.5 g, 5.3 mmol, 1.0 equiv) according to the procedure of example 1 as colorless liquid (1.9 mmol, 1.1 g, 35% yield).

¹H NMR (400 MHz, CDCl₃) δ 7.05 (d, J=8.5 Hz, 4H), 6.78 (d, J=8.5 Hz, 4H), 5.64-5.43 (m, 2H), 4.24-4.02 (m, 2H), 2.87-2.77 (m, 4H), 2.75-2.64 (m, 4H), 2.43-2.23 (m, 4H), 2.11 (s, 6H), 1.69-1.53 (m, 4H), 1.37-1.21 (m, 16H), 0.90-0.82 (m, 6H) ppm. ¹³C NMR (101 MHz, CDCl₃) δ 208.0, 156.8, 132.9, 129.2, 128.6, 116.1, 77.7, 45.4, 37.0, 33.7, 31.8, 30.1, 29.4, 28.9, 25.4, 22.6, 14.1 ppm. GC/MS (EI): m/z (%): 576 (1) [M⁺], 412 (14), 355 (21), 261 (21), 203 (50), 164 (27), 107 (100), 97 (30), 55 (86).

Odour description (1% solution in EtOH on paper blotter, 24 h): powdery fruity raspberry

### Example 11: 3-ethoxy-4-((5-methylocta-1,5-dien-4-yl)oxy)benzaldehyde

The compound was obtained from 3-ethoxy-4-hydroxybenzaldehyde (5.0 g, 30.1 mmol, 1.0 equiv) and (E)-5-methylocta-1,5-dien-4-ol (7.9 g, 39.1 mmol, 1.2 equiv) according to the procedure of example 1 as colorless liquid (18.0 mmol, 5.2 g, 60% yield).

¹H NMR (400 MHz, CDCl₃, mixture of E/Z isomers) δ 9.81 (s, 1H), 7.41-7.31 (m, 2H), 6.96 (d, J=8.2 Hz, 1H), 5.95-5.69 (m, 1H), 5.52-5.37 (m, 1H), 5.20-4.86 (m, 2H), 4.67-4.46 (m, 1H), 4.22-4.03 (m, 2H), 2.88-2.37 (m, 2H), 2.09-1.72 (m, 2H), 1.61 (s, 3H), 1.45 (t, J=7.0 Hz, 3H), 1.02-0.87 (m, 3H) ppm. ¹³C NMR (101 MHz, CDCl₃, mixture of E/Z isomers) δ 191.0, 154.1, 153.9, 150.0, 150.0, 136.2, 134.7, 134.3, 132.2, 131.3, 130.2, 130.1, 126.1, 126.1, 125.9, 117.2, 115.5, 115.2, 114.8, 111.7, 111.6, 86.6, 85.0, 64.7, 64.6, 38.5, 31.7, 26.8, 20.8, 14.8, 13.9, 11.1, 11.0, 10.2 ppm. GC/MS (EI): m/z (%): 288 (1) [M⁺], 247 (2), 189 (3), 166 (6), 137 (15), 122 (35), 93 (31), 81 (100).

Odour description (1% solution in EtOH on paper blotter, 24 h): powdery (vanilla)

### Example 12: (Z)-4-(deca-1,7-dien-4-yloxy)-3-ethoxybenzaldehyde

The compound was obtained from 3-ethoxy-4-hydroxybenzaldehyde (10.0 g, 60.2 mmol, 1.0 equiv) and (Z)-deca-1,7-dien-4-ol (10.2 g, 66.12 mmol, 1.1 equiv) according to the procedure of example 1 as colorless liquid (29.4 mmol, 8.9 g, 48% yield).

¹H NMR (400 MHz, CDCl₃) δ 9.74 (s, 1H), 7.31 (s, 2H), 6.89 (d, J=8.6 Hz, 1H), 5.98-5.60 (m, 1H), 5.39-5.17 (m, 2H), 5.13-4.93 (m, 2H), 4.39-4.20 (m, 1H), 4.04 (q, J=6.8 Hz, 2H), 2.52-2.27 (m, 2H), 2.19-2.04 (m, 2H), 1.99-1.81 (m, 2H), 1.80-1.57 (m, 2H), 1.36 (t, J=6.9 Hz, 3H), 0.80 (t, J=7.5 Hz, 3H) ppm. ¹³C NMR (101 MHz, CDCl₃) δ 189.8, 153.0, 149.1, 132.7, 131.6, 129.2, 126.9, 125.2, 116.8, 113.5, 110.6, 77.5, 63.5, 37.2, 32.6, 21.8, 19.4, 13.7, 13.2 ppm. GC/MS (EI): m/z (%): 302 (12) [M⁺], 193 (1), 166 (100), 138 (51), 95 (23), 81 (20), 67 (19), 55 (13).

Odour description (1% solution in EtOH on paper blotter, 24 h): powdery (vanilla)

### Example 13: 2-methoxy-1-(((Z)-nona-1,6-dien-4-yl)oxy)-4-(prop-1-en-1-yl)benzene

The compound was obtained from 2-methoxy-4-(prop-1-en-1-yl)phenol (0.6 g, 3.7 mmol, 1.3 equiv) and (Z)-nona-1,6-dien-4-ol (0.4 g, 2.9 mmol, 1.0 equiv) according to the procedure of example 1 as colorless liquid (1.0 mmol, 0.3 g, 37% yield).

¹H NMR (400 MHz, CDCl₃): δ=6.88 (s, 1H), 6.80-6.87 (m, 2H), 6.33 (br d, J=15.8 Hz, 1H), 6.07-6.16 (m, 1H), 5.84-5.97 (m, 1H), 5.38-5.53 (m, 2H), 5.03-5.15 (m, 2H), 4.24 (quin, J=5.8 Hz, 1H), 3.84 (s, 3H), 2.37-2.51 (m, 4H), 2.03-2.06 (m, 2H), 1.86 (d, J=6.5 Hz, 3H), 0.95 (t, J=7.4 Hz, 3H) ppm. ¹³C NMR (101 MHz, CDCl₃) δ 150.7, 146.7, 134.6, 134.1, 132.0, 130.6, 124.0, 124.0, 118.6, 117.3, 117.0, 109.6, 79.4, 55.9, 38.1, 31.4, 20.8, 18.4, 14.2 ppm. GC/MS (EI): m/z (%): 286 (10) [M⁺], 217 (1), 164 (100), 149 (11), 131 (6), 103 (8), 91 (11), 81 (7).

Odour description (1% solution in EtOH on paper blotter, 24 h): creamy, vanilla

### Example 14: 4-(4-(((1S,2S,5R)-5-methyl-2-(prop-1-en-2-yl)cyclohexyl)oxy)phenyl)butan-2-one

The compound was obtained from 4-(4-hydroxyphenyl)butan-2-one (5.0 g, 30.4 mmol, 1.0 equiv) and (1R,2S,5R)-5-methyl-2-(prop-1-en-2-yl)cyclohexan-1-ol (5.6 g, 36.5 mmol, 1.2 equiv) according to the procedure of example 1 as white solid (9.6 mmol, 2.9 g, 32% yield). ¹H NMR (400 MHz, CDCl₃) δ 7.05 (d, J=8.5 Hz, 2H), 6.79 (d, J=8.6 Hz, 2H), 4.78 (s, 2H), 4.55 (d, J=2.1 Hz, 1H), 2.86-2.78 (m, 2H), 2.76-2.66 (m, 2H), 2.16-2.04 (m, 5H), 2.03-1.89 (m, 1H), 1.86-1.60 (m, 6H), 1.13-0.93 (m, 2H), 0.84 (d, J=6.5 Hz, 3H) ppm. ¹³C NMR (101 MHz, CDCl₃) δ 208.3, 156.6, 147.3, 132.7, 129.2, 116.1, 110.9, 75.1, 48.0, 45.5, 37.9, 34.8, 30.1, 28.9, 26.1, 25.0, 22.3, 22.0 ppm. GC/MS (EI): m/z (%): 300 (23) [M⁺], 164 (100), 136 (45), 121 (35), 107 (75), 95 (26), 81 (54).

Odour description (1% solution in EtOH on paper blotter, 24 h): sweet fruity (raspberry)

### Example 15: 3-ethoxy-4-((1-phenylnon-1-en-4-yl)oxy)benzaldehyde

The compound was obtained from 3-ethoxy-4-hydroxybenzaldehyde (0.3 g, 1.9 mmol, 1.3 equiv) and 1-phenylnon-1-en-4-ol (0.4 g, 1.5 mmol, 1.0 equiv) according to the procedure of example 1 as colorless liquid (0.3 mmol, 0.12 g, 21% yield).

¹H NMR (400 MHz, CDCl₃): δ=9.83 (s, 1H), 7.39-7.43 (m, 2H), 7.19-7.35 (m, 5H), 7.01 (d, J=8.1 Hz, 1H), 6.47 (d, J=15.8 Hz, 1H), 6.18-6.34 (m, 1H), 4.42-4.46 (m, 1H), 4.13 (q, J=6.8 Hz, 2H), 2.55-2.70 (m, 2H), 1.25-1.86 (m, 11H), 0.88 (br t, J=6.0 Hz, 3H) ppm. ¹³C NMR (101 MHz, CDCl₃) δ 191.0, 154.1, 150.2, 137.4, 132.8, 130.2, 128.5, 127.3, 126.3, 126.1, 125.6, 114.9, 111.7, 79.9, 64.6, 37.6, 33.8, 31.8, 25.1, 22.6, 14.8, 14.0 ppm. GC/MS (EI): m/z (%): 366 (1) [M⁺], 249 (11), 201 (33), 165 (28), 117 (100), 91 (61).

Odour description (1% solution in EtOH on paper blotter, 24 h): powdery (vanilla)

### Example 16: 4-((2,6-dimethylhepta-4,5-dien-2-yl)oxy)-3-ethoxybenzaldehyde

The compound was obtained from 3-ethoxy-4-hydroxybenzaldehyde (1.5 g, 9.0 mmol, 1.0 equiv) and 2,2,5-trimethylhexa-3,4-dien-1-ol (1.3 g, 9.0 mmol, 1.0 equiv) according to the procedure of example 1 as colorless liquid (0.1 mmol, 0.04 g, 1% yield).

¹H NMR (400 MHz, CDCl₃): δ=9.88 (s, 1H), 7.40 (d, J=1.6 Hz, 1H), 7.38 (dd, J=8.0, 1.9 Hz, 1H), 7.16 (d, J=8.0 Hz, 1H), 5.00-5.21 (m, 1H), 4.09 (q, J=6.9 Hz, 2H), 2.39 (d, J=7.6 Hz, 2H), 1.64-1.74 (m, 6H), 1.43-1.52 (m, 3H), 1.36 (s, 6H) ppm. ¹³C NMR (101 MHz, CDCl₃) δ=203.7, 191.3, 153.9, 151.2, 132.4, 124.9, 124.5, 111.5, 94.2, 84.5, 83.6, 64.2, 42.7, 26.0, 20.5, 14.7 ppm. GC/MS (EI): m/z (%): 288 (6) [M⁺], 166 (33), 137 (40), 123 (26), 107 (100), 81 (91), 67 (30).

Odour description (1% solution in EtOH on paper blotter, 24 h): powdery (vanilla)

### Example 17: (Z)-3-ethoxy-4-(undec-3-en-6-yloxy)benzaldehyde

The compound was obtained from 3-ethoxy-4-hydroxybenzaldehyde (0.5 g, 3.1 mmol, 1.3 equiv) and (Z)-undec-3-en-6-ol (0.4 g, 2.3 mmol, 1.0 equiv) according to the procedure of example 1 as colorless liquid (0.9 mmol, 0.3 g, 39% yield).

¹H NMR (400 MHz, CDCl₃): δ=9.83 (s, 1H), 7.40 (s, 1H), 7.41 (d, J=6.3 Hz, 1H), 6.97 (d, J=8.6 Hz, 1H), 5.36-5.54 (m, 2H), 4.39-4.33 (m 1H), 4.13 (q, J=6.9 Hz, 2H), 2.38-2.54 (m, 2H), 2.10-2.02 (m, 2H), 1.67-1.81 (m, 2H), 1.42-1.52 (m, 4H), 1.26-1.34 (m, 5H), 0.86-1.00 (m, 6H) ppm. ¹³C NMR (101 MHz, CDCl₃) δ=191.0, 154.2, 150.1, 134.4, 130.0, 126.3, 123.7, 114.4, 111.7, 79.7, 64.6, 33.7, 31.8, 31.6, 25.0, 22.6, 20.8, 14.7, 14.1, 14.0 ppm. GC/MS (EI): m/z (%): 318 (6) [M⁺], 249 (2), 166 (100), 138 (47), 97 (26), 83 (21), 69 (34), 55 (36).

Odour description (1% solution in EtOH on paper blotter, 24 h): powdery (dry, vanilla, chocolate)

### Example 18: 3-ethoxy-4-((2,6,10-trimethylundeca-4,5,9-trien-2-yl)oxy)benzaldehyde

The compound was obtained from 3-ethoxy-4-hydroxybenzaldehyde (7.3 g, 43.7 mmol, 1.3 equiv) and 2,2,5,9-tetramethyldeca-3,4,8-trien-1-ol (7.0 g, 33.6 mmol, 1.0 equiv) according to the procedure of example 1 as colorless liquid (3.1 mmol, 1.1 g, 9% yield).

¹H NMR (400 MHz, CDCl₃): δ=9.88 (s, 1H), 7.40-7.36 (m, 2H), 7.16 (d, J=7.9 Hz, 1H), 5.10-5.23 (m, 2H), 4.09 (q, J=7.0 Hz, 2H), 2.41 (dd, J=7.6, 1.6 Hz, 2H), 2.13-1.93 (m, 4H), 1.68-1.71 (m, 6H), 1.59 (s, 3H), 1.41-1.53 (m, 3H), 1.37 (s, 6H) ppm. ¹³C NMR (101 MHz, CDCl₃) δ 203.2, 191.3, 153.9, 151.1, 132.4, 131.6, 124.9, 124.5, 124.2, 111.5, 98.5, 86.0, 83.6, 64.2, 42.9, 34.1, 26.3, 26.1, 25.7, 19.1, 17.7, 14.7 ppm. GC/MS (EI): m/z (%): 356 (1) [M⁺], 313 (2), 190 (53), 175 (62), 137 (100), 107 (69), 93 (90), 79 (49).

Odour description (1% solution in EtOH on paper blotter, 24 h): vanilla powdery

### Example 19: (E)-3-ethoxy-4-(non-6-en-4-yloxy)benzaldehyde

### a) (E)-non-6-en-4-ol

To a solution of (E)-hex-3-enal (12.5 g, 127.0 mmol, 1.0 equiv) in THF (150 mL) was added propylmagnesium bromide (37.5 g, 255.0 mmol, 2.0 equiv) dropwise in 0° C. Stir at rt for 4 h. Water (15 mL) was added to quench the reaction. Filter off the solid and purity the product by silica gel chromatography to give (E)-non-6-en-4-ol as colorless liquid (21.8 mmol, 3.1 g, 15% yield).

¹³C NMR (101 MHz, CDCl₃) δ 136.2, 124.9, 70.7, 40.7, 38.9, 25.7, 18.9, 14.1, 13.8. GC/MS (EI): m/z (%): 142 (1) [M⁺], 124 (2), 114 (1), 100 (2), 81 (8), 70 (72), 55 (100).

### b) (E)-3-ethoxy-4-(non-6-en-4-yloxy)benzaldehyde

The compound was obtained from 3-ethoxy-4-hydroxybenzaldehyde (0.2 g, 1.0 mmol, 1.3 equiv) and (E)-non-6-en-4-ol (0.1 g, 0.7 mmol, 1.0 equiv) according to the procedure of example 1 as colorless liquid (0.2 mmol, 0.07 g, 33% yield).

¹H NMR (400 MHz, CDCl₃): δ=9.83 (s, 1H), 7.40 (s, 1H), 7.41 (d, J=6.2 Hz, 1H), 6.98 (d, J=8.8 Hz, 1H), 5.51-5.61 (m, 1H), 5.38-5.50 (m, 1H), 4.35 (quin, J=5.9 Hz, 1H), 4.13 (q, J=6.9 Hz, 2H), 2.32-2.48 (m, 2H), 1.94-2.08 (m, 2H), 1.59-1.83 (m, 2H), 1.35-1.56 (m, 5H), 0.90-0.99 (m, 6H) ppm. ¹³C NMR (101 MHz, CDCl₃) δ 191.0, 154.4, 150.1, 135.5, 130.0, 126.3, 124.0, 114.6, 111.9, 79.7, 64.7, 37.1, 35.8, 25.7, 18.6, 14.7, 14.1, 13.7 ppm. GC/MS (EI): m/z (%): 290 (17) [M⁺], 221 (4), 166 (100), 138 (56), 83 (35), 69 (77), 55 (38).

Odour description (1% solution in EtOH on paper blotter, 24 h): vanilla powdery creamy

### Example 20: 3-ethoxy-4-(((3E,7E)-4,8,12-trimethyltrideca-3,7,11-trien-1-yl)oxy)benzaldehyde

The compound was obtained from 3-ethoxy-4-hydroxybenzaldehyde (0.4 g, 2.4 mmol, 1.1 equiv) and (3E,7E)-4,8,12-trimethyltrideca-3,7,11-trien-1-ol (0.2 g, 2.2 mmol, 1.0 equiv) according to the procedure of example 1 as colorless liquid (1.8 mmol, 0.7 g, 78% yield).

¹H NMR (400 MHz, CDCl₃, mixture of E/Z isomers) δ 9.83 (s, 1H), 7.52-7.31 (m, 2H), 7.07-6.81 (m, 1H), 5.22 (t, J=7.3 Hz, 1H), 5.15-5.02 (m, 2H), 4.14 (q, J=7.0 Hz, 2H), 4.09-4.00 (m, 2H), 2.59 (q, J=7.3 Hz, 2H), 2.19-2.00 (m, 6H), 2.00-1.91 (m, 2H), 1.79-1.72 (m, 1H), 1.71-1.64 (m, 5H), 1.63-1.53 (m, 6H), 1.52-1.41 (m, 3H) ppm. ¹³C NMR (101 MHz, CDCl₃, mixture of E/Z isomers) δ 190.9, 154.4, 154.4, 149.2, 138.8, 138.7, 135.4, 135.1, 131.3, 131.3, 129.9, 126.6, 124.3, 124.3, 124.0, 123.9, 119.5, 118.7, 111.7, 110.9, 68.8, 68.6, 64.5, 39.7, 39.7, 32.1, 27.9, 27.8, 26.8, 26.7, 26.5, 25.7, 23.5, 17.7, 16.2, 16.0, 14.7 ppm. GC/MS (EI): m/z (%): 384 (1) [M⁺], 248 (4), 219 (2), 166 (5), 137 (16), 95 (18), 81 (51), 69 (100).

Odour description (1% solution in EtOH on paper blotter, 24 h): vanilla (powdery, creamy, chocolate, gourmand)

### Example 21: 3-ethoxy-4-((2-methyl-1-phenylhex-4-en-2-yl)oxy)benzaldehyde

### a) (E)-2-benzyl-2-methylpent-3-en-1-ol

To a suspension of LiAlH₄ (3.0 g, 80.0 mmol, 0.75 equiv) in THF (200 mL) was added (E)-2-benzyl-2-methylpent-3-enal (20.0 g, 106.0 mmol, 1.0 equiv) in Tetrahydrofuran (100 ml) at 5° C. dropwise and the resulting mixture was stirred at r.t. for 3 h. Then reaction was quenched by adding some solution of NH4Cl, the mixture was extracted with MTBE (150 mL*3), the organic layers were combined, and dried over MgSO₄ and the solvent was removed to give (E)-2-benzyl-2-methylpent-3-en-1-ol (17.2 g, 90.4 mmol, 85% yield).

¹³C NMR (101 MHz, CDCl₃) δ 138.2, 136.6, 130.7, 128.6, 127.7, 127.0, 126.0, 125.1, 69.4, 43.7, 42.3, 20.8, 18.4. GC/M S (EI): m/z (%): 190 (2) [M⁺], 172 (18), 159 (17), 129 (22), 117 (55), 91 (100), 65 (32), 53 (12).

### b) 3-ethoxy-4-((2-methyl-1-phenylhex-4-en-2-yl)oxy)benzaldehyde

The compound was obtained from 3-ethoxy-4-hydroxybenzaldehyde (4.8 g, 28.9 mmol, 1.1 equiv) and (E)-2-benzyl-2-methylpent-3-en-1-ol (5.0 g, 26.3 mmol, 1.0 equiv) according to the procedure of example 1 as light yellow liquid (0.6 mmol, 0.2 g, 2% yield).

¹H NMR (400 MHz, CDCl₃) δ 9.76 (s, 1H), 7.29-6.75 (m, 8H), 5.76-5.26 (m, 2H), 3.97 (q, J=7.0 Hz, 2H), 3.17-2.82 (m, 2H), 2.98-2.11 (m, 2H), 1.74-1.55 (m, 3H), 1.41-1.29 (m, 3H), 1.11 (s, 3H) ppm. ¹³C NMR (101 MHz, CDCl₃) δ 191.2, 153.8, 151.2, 137.7, 132.2, 131.0, 128.8, 127.9, 126.5, 126.4, 124.9, 124.0, 111.2, 85.4, 64.2, 46.1, 42.8, 23.5, 18.2, 14.8 ppm. GC/MS (EI): m/z (%): 338 (1) [M⁺], 283 (1), 172 (22), 157 (14), 143 (30), 91 (100), 55 (19).

Odour description (1% solution in EtOH on paper blotter, 24 h): powdery (vanilla) creamy (milky).

### Example 22: 4-(dec-1-en-4-yloxy)-3-ethoxybenzaldehyde

The compound was obtained from 3-ethoxy-4-hydroxybenzaldehyde (4.7 g, 28.2 mmol, 1.1 equiv) and dec-1-en-4-ol (4.0 g, 25.6 mmol, 1.0 equiv) according to the procedure of example 1 as colorless liquid (12.8 mmol, 3.9 g, 50% yield).

¹H NMR (400 MHz, CDCl₃) δ 9.83 (s, 1H), 7.46-7.29 (m, 2H), 6.99 (d, J=8.4 Hz, 1H), 6.07-5.70 (m, 1H), 5.27-4.94 (m, 2H), 4.50-4.29 (m, 1H), 4.13 (q, J=6.9 Hz, 2H), 2.62-2.29 (m, 2H), 1.89-1.59 (m, 2H), 1.49-1.28 (m, 11H), 0.98-0.67 (m, 3H) ppm. ¹³C NMR (101 MHz, CDCl₃) δ 190.9, 154.1, 150.1, 133.9, 130.1, 126.3, 117.7, 114.7, 111.7, 79.4, 64.6, 38.3, 33.6, 31.7, 29.2, 25.2, 22.5, 14.7, 14.1 ppm. GC/MS (EI): m/z (%): 304 (5) [M⁺], 263 (1), 166 (100), 138 (56), 81 (8), 55 (18).

Odour description (1% solution in EtOH on paper blotter, 24 h): dry powdery (cedarwood, vanilla)

### Example 23: 4-(4-(dec-1-en-4-yloxy)phenyl)butan-2-one

The compound was obtained from 4-(4-hydroxyphenyl)butan-2-one (4.6 g, 28.2 mmol, 1.1 equiv) and dec-1-en-4-ol (4.0 g, 25.6 mmol, 1.0 equiv) according to the procedure of example 1 as colorless liquid (13.9 mmol, 4.2 g, 55% yield).

¹H NMR (400 MHz, CDCl₃) δ 7.07 (d, J=7.8 Hz, 2H), 6.81 (d, J=7.9 Hz, 2H), 5.95-5.68 (m, 1H), 5.15-5.05 (m, 2H), 4.29-4.09 (m, 1H), 2.83 (t, J=7.4 Hz, 2H), 2.72 (t, J=7.1 Hz, 2H), 2.44-2.35 (m, 2H), 2.13 (s, 3H), 1.74-1.52 (m, 2H), 1.44-1.27 (s, 8H), 0.87 (t, J=5.7 Hz, 3H) ppm. ¹³C NMR (101 MHz, CDCl₃) δ 208.2, 156.8, 134.3, 133.0, 129.2, 117.3, 116.1, 77.5, 45.5, 38.2, 33.7, 31.8, 30.1, 29.3, 28.9, 25.4, 22.6, 14.1 ppm. GC/MS (EI): m/z (%): 302 (12) [M⁺], 261 (6), 203 (5), 164 (100), 149 (12), 107 (90), 94 (27), 55 (18).

Odour description (1% solution in EtOH on paper blotter, 24 h): fruity (raspberry, gourmand) sugary

### Example 24: methyl 4-(dec-1-en-4-yloxy)-2-hydroxy-3,6-dimethylbenzoate

The compound was obtained from methyl 2,4-dihydroxy-3,6-dimethylbenzoate (5.5 g, 28.2 mmol, 1.1 equiv) and dec-1-en-4-ol (4.0 g, 25.6 mmol, 1.0 equiv) according to the procedure of example 1 as colorless liquid (14.7 mmol, 4.9 g, 57% yield).

¹H NMR (400 MHz, CDCl₃) δ 11.88 (s, 1H), 6.25 (s, 1H), 5.91-5.64 (m, 1H), 5.29-4.89 (m, 2H), 4.51-4.25 (m, 1H), 3.90 (s, 3H), 2.49 (s, 3H), 2.46-2.27 (m, 2H), 2.08 (s, 3H), 1.75-1.56 (m, 2H), 1.36-1.19 (m, 8H), 0.86 (s, 3H) ppm. ¹³C NMR (101 MHz, CDCl₃) δ 172.6, 162.6, 160.4, 139.8, 134.0, 117.5, 111.9, 108.0, 105.0, 77.2, 51.7, 38.4, 33.7, 31.8, 29.3, 25.2, 24.7, 22.6, 14.1, 8.1 ppm. GC/MS (EI): m/z (%): 334 (6) [M⁺], 293 (2), 196 (25), 164 (100), 136 (19), 55 (11).

Odour description (1% solution in EtOH on paper blotter, 24 h): powdery (mossy, evernyl)

### Example 25: methyl 2-(tetradec-1-en-4-yloxy)benzoate

The compound was obtained from methyl salicylate (1.3 ml, 10 mmol, 1 equiv.) and tetradec-1-en-4-ol (2.12 g, 10 mmol, 1 equiv.) according to the procedure of example 1 as a clear, colorless liquid (1.56 g, 4.4 mmol, 44%).

¹H NMR (400 MHz, CDCl₃, 298 K) δ (ppm)=7.83-7.67 (m, 1H), 7.53-7.36 (m, 1H), 7.05-6.87 (m, 2H), 6.03-5.80 (m, 1H), 5.19-5.01 (m, 2H), 4.45-4.28 (m, 1H), 3.87 (s, 3H), 2.63-2.37 (m, 2H), 1.86-1.60 (m, 2H), 1.52-1.16 (m, 16H), 0.95-0.80 (m, 3H). ¹³C NMR (101 MHz, CDCl₃, 298 K) δ (ppm)=167.2, 157.8, 134.2, 133.0, 131.6, 121.7, 120.0, 117.4, 114.8, 78.7, 51.8, 38.2, 33.5, 31.9, 29.6, 29.6, 29.6, 29.6, 29.3, 25.2, 22.7, 14.1. MS (EI, 70 eV): 346 (1, [M]+*), 305 (5), 152 (100), 120 (75), 92 (15), 81 (10), 67 (12), 55 (20), 41 (25), 29 (10). Odour description (1% solution in EtOH on paper blotter, 24 h): aldehydic, metallic, green, citrus.

### Example 26: 1-(2-(tetradec-1-en-4-yloxy)phenyl)propan-1-one

The compound was obtained from 1-(2-hydroxyphenyl)propan-1-one (1.37 ml, 10 mmol, 1 equiv.) and tetradec-1-en-4-ol (2.12 g, 10 mmol, 1 equiv.) according to the procedure of example 1 as clear colorless liquid (1.12 g, 2.8 mmol, 28%).

¹H NMR (400 MHz, CDCl3, 298 K) δ (ppm)=7.66 (dd, J=1.8, 7.6 Hz, 1H), 7.48-7.20 (m, 1H), 7.02-6.82 (m, 2H), 5.82 (br dd, J=10.2, 17.1 Hz, 1H), 5.20-5.01 (m, 2H), 4.55-4.35 (m, 1H), 2.99 (q, J=7.2 Hz, 2H), 2.61-2.40 (m, 2H), 1.79-1.64 (m, 2H), 1.53-1.23 (m, 16H), 1.20-1.13 (m, 3H), 0.97-0.81 (m, 3H). ¹³C NMR (101 MHz, CDCl3, 298 K) δ (ppm)=204.0, 156.9, 133.7, 132.9, 130.5, 129.5, 120.3, 117.9, 113.1, 77.3, 38.0, 37.2, 33.4, 31.9, 29.6, 29.6, 29.6, 29.5, 29.3, 25.3, 22.7, 14.1, 8.6. MS (EI, 70 eV): 344 (1, [M]+·), 303 (5), 150 (70), 133 (2), 121 (100), 150 (2), 97 (4), 83 (6), 67 (7), 55 (20), 41 (25), 29 (7).

Odour description (1% solution in EtOH on paper blotter, 24 h): aldehydic, metallic, green, citrus.

### Example 27: 3-ethoxy-4-(tetradec-1-en-4-yloxy)benzaldehyde

The compound was obtained from ethyl vanillin (4.15 g, 25 mmol, 1 equiv.) and tetradec-1-en-4-ol (5.31 g, 25 mmol, 1 equiv.) according to the procedure of example 1 as clear colorless liquid (2.23 g, 5.9 mmol, 24%).

¹H NMR (400 MHz, CDCl₃, 298 K) δ (ppm)=9.83 (s, 1H), 7.43-7.38 (m, 2H), 6.98 (d, J=8.7 Hz, 1H), 5.87 (tdd, J=7.0, 10.1, 17.1 Hz, 1H), 5.18-5.05 (m, 2H), 4.38 (quin, J=5.9 Hz, 1H), 4.13 (q, J=7.0 Hz, 2H), 2.56-2.39 (m, 2H), 1.81-1.63 (m, 2H), 1.53-1.19 (m, 19H), 0.92-0.84 (m, 3H). ¹³C NMR (101 MHz, CDCl3, 298 K) δ (ppm)=190.9, 154.1, 150.1, 133.9, 130.2, 126.2, 117.7, 114.7, 111.8, 79.5, 64.7, 38.3, 33.7, 31.9, 29.6, 29.6, 29.5, 29.5, 29.3, 25.3, 22.7, 14.7, 14.1. MS (EI, 70 eV): 360 (1, [M]+·), 194 (1), 166 (100), 149 (3) 138 (40), 123 (1), 109 (5), 97 (4), 81 (5), 69 (5), 55 (10), 41 (12), 29 (6).

Odour description (1% solution in EtOH on paper blotter, 24 h): powdery, vanilla, aldehydic, metallic, green, citrus, mandarin.

### Example 28: (Z)-3-ethoxy-4-(Pentadec-3-en-6-yloxy)benzaldehyde

### a) pentadec-3-yn-6-ol

A solution of Butyllithium (1.6 M in hexanes, 62 mL, 99 mmol) was mixed with THF (tetrahydrofuran) (150 mL) and cooled to −78° C. Gaseous but-1-yne (8.94 g, 165 mmol) was slowly bubbled through this solution at −78° C. over 15 min and the resulting mixture stirred for additional 15 min. The mixture was then treated slowly at −78° C. with 2-nonyloxirane (9.38 g, 55 mmol), followed by borontrifluoride etherate (5.7 mL, 45 mmol) and the resulting mixture stirred at −78° C. for 1.5 h. The mixture was finally poured onto sat. aq. NaHCO₃ soln. (300 mL), diluted with water (200 mL), extracted with MTBE (2×200 mL), washed with brine (100 mL), dried over MgSO₄ and concentrated to give a crude that was purified by Kugelrohr distillation at 180° C. and 0.12 mbar followed by flash chromatography on silica gel eluting with a gradient of MTBE in heptane to give pentadec-3-yn-6-ol (8.58 g, 69% yield) as a pale yellow oil that was used without further purification in the next step.

MS (EI, 70 eV): 224 (1, [M]+·), 209 (2), 195 (2), 157 (6) 125 (4), 97 (42), 68 (100), 67 (84).

### b) pentadec-3-en-6-ol

A flask was charged with Lindlar catalyst (Palladium on calcium carbonate, poisoned with lead) (1.2 g), ethyl acetate (300 mL), quinoline (30 mL) and pentadec-3-yn-6-ol (7.35 g, 32.8 mmol) and the flask purged with nitrogen followed by hydrogen. The mixture was allowed to stir under hydrogen for 1 h at room temperature after which it was flushed with nitrogen and filtered through a plug of celite, rinsing with MTBE (methyl tert-butyl ether). The mixture was extracted with 2 M HCl (2×100 mL) and the aqueous layer extracted with additional MTBE (2×50 mL). the combined organic layers were dried over MgSO₄ and evaporated to give pentadec-3-en-6-ol (7.51 g, 97% purity, 98% yield) as a pale yellow oil that was used in the next step without purification.

MS (EI, 70 eV): 226 (1, [M]+·), 208 (2), 184 (1), 157 (7) 70 (100), 69 (34).

### c) (Z)-3-ethoxy-4-(pentadec-3-en-6-yloxy)benzaldehyde

The compound was obtained from pentadec-3-en-6-ol (8.75 g, 38.6 mmol), ethyl vanillin (3-ethoxy-4-hydroxybenzaldehyde) (6.42 g, 38.6 mmol), triphenylphosphine (15.21 g, 58 mmol) and DIAD (diisopropyl azodicarboxylate) (11.7 g, 58 mmol) according to the process of example 1 as a colorless oil (3.74 g, 26% yield).

¹H NMR (400 MHz, CDCl3, 298 K) δ (ppm)=9.85 (s, 1H), 7.40-7.45 (m, 2H), 6.99 (d, J=8.6 Hz, 1H), 5.47-5.57 (m, 1H), 5.37-5.47 (m, 1H), 4.38 (quin, J=5.9 Hz, 1H), 4.15 (q, J=6.8 Hz, 2H), 2.40-2.56 (m, 2H), 2.03-2.13 (m, 2H), 1.64-1.84 (m, 2H), 1.22-1.53 (m, 17H), 0.98 (t, J=7.6 Hz, 3H), 0.86-0.92 (m, 3H). ¹³C NMR (101 MHz, CDCl3, 298 K) δ (ppm)=190.9, 154.2, 150.1, 134.3, 130.0, 126.3, 123.7, 114.4, 111.8, 79.7, 64.6, 33.7, 31.9, 31.6, 29.6, 29.6, 29.5, 29.3, 25.3, 22.7, 20.8, 14.7, 14.1, 14.1. MS (EI, 70 eV): 374 (1, [M]+·), 167 (21), 166 (100), 138 (38), 137 (35), 97 (23), 83 (28), 69 (36), 55 (32), 43 (23), 41 (34).

Odour description (1% solution in EtOH on paper blotter, 24 h): powdery, creamy, vanilla, aldehydic, mandarin.

### Example 29: (E)-3-ethoxy-4-(pentadec-3-en-6-yloxy)benzaldehyde

### a) 3-ethoxy-4-(pentadec-3-yn-6-yloxy)benzaldehyde

The compound was obtained from pentadec-3-yn-6-ol (2.24 g, 10 mmol), ethyl vanillin (3-ethoxy-4-hydroxybenzaldehyde) (1.66 g, 10 mmol), triphenylphosphine (3.93 g, 15 mmol) and DIAD (diisopropyl azodicarboxylate) (3.03 g, 15 mmol) according to the procedure of example 1 as a colorless oil (1.17 g, 31% yield).

MS (EI, 70 eV): 372 (2, [M]+·), 166 (100), 138 (40).

### b) (E)-3-ethoxy-4-(pentadec-3-en-6-yloxy)benzaldehyde

A flask was charged with Lindlar catalyst (Palladium on calcium carbonate, poisoned with lead) (100 mg), toluene (30 mL), pyridine (3 mL) and 3-ethoxy-4-(pentadec-3-yn-6-yloxy)benzaldehyde (1.17 g, 3.14 mmol) and the flask purged with nitrogen followed by hydrogen. The mixture was allowed to stir under hydrogen for 3 h at room temperature after which it was flushed with nitrogen and filtered through a plug of celite, rinsing with toluene. The solvent was evaporated and the resulting material purified by chromatography on silica gel, eluting with a gradient of ethyl acetate in heptane to give (E)-3-ethoxy-4-(pentadec-3-en-6-yloxy)benzaldehyde (0.55 g, 97% purity, 45% yield) as a colorless oil.

¹H NMR (400 MHz, CDCl3, 298 K) δ (ppm)=9.84 (s, 1H), 7.44-7.40 (m, 2H), 6.99 (d, J=8.6 Hz, 1H), 5.63-5.54 (m, 1H), 5.51-5.42 (m, 1H), 4.35 (quin, J=5.9 Hz, 1H), 4.15 (q, J=7.1 Hz, 2H), 2.52-2.33 (m, 2H), 2.06-1.98 (m, 2H), 1.81-1.62 (m, 2H), 1.52-1.22 (m, 17H), 0.97 (t, J=7.5 Hz, 3H), 0.92-0.86 (m, 3H). ¹³C NMR (101 MHz, CDCl3, 298 K) δ (ppm)=190.9, 154.3, 150.0, 135.4, 129.8, 126.3, 124.0, 114.6, 114.2, 111.9, 80.0, 64.7, 33.9, 33.9, 31.9, 29.6, 29.6, 29.5, 29.5, 25.3, 25.0, 22.7, 14.7, 14.1, 13.7. MS (EI, 70 eV): 374 (1, [M]+·), 208 (3), 167 (18), 166 (100), 138 (39), 137 (22), 55 (32), 43 (28), 41 (30).

Odour description (1% solution in EtOH on paper blotter, 24 h): powdery, creamy, vanilla, aldehydic, mandarin.

### Example 30: 3-ethoxy-4-(((3Z,6Z)-nona-3,6-dien-1-yl)oxy)benzaldehyde

The compound was obtained from (3Z,6Z)-nona-3,6-dien-1-ol (7.00 g, 49.9 mmol), ethyl vanillin (3-ethoxy-4-hydroxybenzaldehyde) (8.30 g, 49.9 mmol), triphenylphosphine (19.64 g, 74.9 mmol) and DIAD (diisopropyl azodicarboxylate) (15.4 g, 74.9 mmol) according to the procedure of example 1 as a colorless oil (4.20 g, 88% purity, 26% yield).

¹H NMR (400 MHz, CDCl3, 298 K) δ (ppm)=9.85 (s, 1H), 7.46-7.40 (m, 2H), 7.01-6.95 (m, 1H), 5.68-5.29 (m, 4H), 4.20-4.07 (m, 4H), 2.87 (t, J=7.1 Hz, 2H), 2.73-2.61 (m, 2H), 2.15-2.00 (m, 2H), 1.49 (t, J=7.0 Hz, 3H), 0.99 (t, J=7.6 Hz, 3H). ¹³C NMR (101 MHz, CDCl3, 298 K) δ (ppm)=190.9, 154.3, 149.2, 132.2, 131.4, 130.0, 126.8, 126.5, 124.3, 111.8, 110.8, 68.4, 64.5, 27.3, 25.7, 20.6, 14.7, 14.2. MS (EI, 70 eV): 288 (1, [M]+·), 137 (22), 122 (21), 93 (53), 81 (100), 79 (42), 69 (34), 67 (55), 55 (39), 41 (35), 29 (25).

Odour description (1% solution in EtOH on paper blotter, 24 h): green, leaf aldehyde, green apple, powdery, vanilla.

### Example 31: 4-(4-(((3Z,6Z)-nona-3,6-dien-1-yl)oxy)phenyl)butan-2-one

The compound was obtained from (3Z,6Z)-nona-3,6-dien-1-ol (1.40 g, 9.98 mmol), raspberry ketone (4-(4-hydroxyphenyl)butan-2-one) (1.64 g, 9.98 mmol), triphenylphosphine (3.93 g, 15.0 mmol) and DIAD (diisopropyl azodicarboxylate) (3.03 g, 15.0 mmol) according to the procedure of example 1 as a colorless oil (1.13 g, 40% yield).

¹H NMR (400 MHz, CDCl3, 298 K) δ (ppm)=7.14-7.08 (m, 2H), 6.87-6.81 (m, 2H), 5.45-5.44 (m, 1H), 5.60-5.29 (m, 3H), 3.96 (t, J=6.8 Hz, 2H), 2.90-2.82 (m, 4H), 2.78-2.71 (m, 2H), 2.62-2.53 (m, 2H), 2.15 (s, 3H), 2.14-2.03 (m, 2H), 1.00 (t, J=7.5 Hz, 3H). ¹³C NMR (101 MHz, CDCl3, 298 K) δ (ppm)=208.2, 157.3, 133.0, 132.1, 130.8, 129.2, 126.9, 125.0, 114.6, 67.4, 45.5, 30.1, 28.9, 27.5, 25.7, 20.6, 14.3. MS (EI, 70 eV): 286 (11, [M]+·), 107 (96), 93 (96), 91 (32), 81 (100), 79 (56), 69 (33), 67 (61), 55 (47), 43 (71), 41 (46).

Odour description (1% solution in EtOH on paper blotter, 24 h): green, leaf aldehyde, green apple, fruity.

### Example 32: 1-methoxy-2-(((3Z,6Z)-nona-3,6-dien-1-yl)oxy)benzene

### a) (3Z,6Z)-1-chloronona-3,6-diene

A solution of (3Z,6Z)-nona-3,6-dien-1-ol (2.80 g, 20.0 mmol) in toluene (40 mL) was treated dropwise at 0° C. with pyridine (0.19 g, 2.4 mmol) and then thionyl chloride (2.85 g, 24.0 mmol). The resulting mixture was heated to 80° C. for 2 h and then cooled to room temperature, poured onto iced aqueous NaHCO₃ solution (150 mL), extracted with MTBE (2×50 mL), dried over MgSO₄, filtered and concentrated. The resulting material was purified by chromatography on silica gel eluting with a gradient of MTBE in heptane, followed by Kugelrohr distillation to give (3Z,6Z)-1-chloronona-3,6-diene (2.69 g, 88% purity, 75% yield) as a pale yellow oil which was used without further purification.

MS (EI, 70 eV): 158 (22, [M]+·), 143 (2), 129 (7), 123 (3), 109 (19), 67 (100).

### b) 1-methoxy-2-(((3Z,6Z)-nona-3,6-dien-1-yl)oxy)benzene

A mixture of guaiacol (2-methoxyphenol) (0.78 g, 6.30 mmol), K₂CO₃ (1.31 g, 9.45 mmol), tetrabutylammonium iodide (0.35 g, 0.945 mmol) and acetonitrile (8 mL) was treated at room temperature with (3Z,6Z)-1-chloronona-3,6-diene (1.00 g, 6.3 mmol) and the resulting mixture stirred overnight then heated to reflux for 4 h. The reaction mixture was then poured into saturated aqueous NH₄Cl solution (50 mL), extracted with MTBE (2×50 mL), dried over MgSO4, filtered and evaporated. The resulting material was purified by chromatography on silica gel eluting with a gradient of MTBE in heptane, followed by Kugelrohr distillation to give 1-methoxy-2-(((3Z,6Z)-nona-3,6-dien-1-yl)oxy)benzene (0.27 g, 17% yield) as a colorless oil. ¹H NMR (400 MHz, CDCl3, 298 K) δ (ppm)=6.99-6.86 (m, 4H), 5.61-5.30 (m, 4H), 4.09-4.01 (m, 2H), 3.92-3.87 (m, 3H), 2.91-2.81 (m, 2H), 2.73-2.61 (m, 2H), 2.16-2.02 (m, 2H), 1.00 (t, J=7.5 Hz, 3H). ¹³C NMR (101 MHz, CDCl3, 298 K) δ (ppm)=149.5, 148.4, 132.2, 131.0, 126.9, 124.6, 121.1, 120.9, 113.3, 111.9, 68.3, 55.9, 27.5, 25.7, 20.6, 14.3. MS (EI, 70 eV): 246 (2, [M]+·), 124 (100), 122 (38), 109 (25), 93 (42), 81 (53), 79 (27), 77 (27), 67 (29), 55 (23), 41 (26).

Odour description (1% solution in EtOH on paper blotter, 24 h): green, leaf aldehyde.

### Example 33: 2-(((3Z,6Z)-nona-3,6-dien-1-yl)oxy)naphthalene

The compound was obtained from naphthalen-2-ol (0.91 g, 6.3 mmol), K₂CO₃ (1.31 g, 9.45 mmol), tetrabutylammonium iodide (0.35 g, 0.945 mmol) and (3Z,6Z)-1-chloronona-3,6-diene (1.00 g, 6.3 mmol) according to the procedure of example 32b as a colorless oil (0.33 g, 20% yield).

¹H NMR (400 MHz, CDCl3, 298 K) δ (ppm)=7.85-7.73 (m, 3H), 7.47 (ddd, J=1.2, 6.8, 8.1 Hz, 1H), 7.37 (ddd, J=1.2, 7.0, 8.1 Hz, 1H), 7.22-7.14 (m, 2H), 5.70-5.33 (m, 4H), 4.18-4.08 (m, 2H), 2.96-2.81 (m, 2H), 2.74-2.60 (m, 2H), 2.20-2.07 (m, 2H), 1.03 (t, J=7.5 Hz, 3H). ¹³C NMR (101 MHz, CDCl3, 298 K) δ (ppm)=156.9, 134.6, 132.2, 131.0, 129.4, 129.0, 127.6, 126.9, 126.7, 126.3, 125.0, 123.6, 119.0, 106.7, 67.4, 27.5, 25.8, 20.6, 14.3. MS (EI, 70 eV): 266 (6, [M]+·), 145 (15), 144 (100), 127 (33), 115 (42), 93 (38), 81 (28), 79 (20), 67 (19), 55 (16), 41 (18).

Odour description (1% solution in EtOH on paper blotter, 24 h): weak, green, leaf aldehyde.

### Example 34: 3-methoxy-4-(((3Z,6Z)-nona-3,6-dien-1-yl)oxy)benzaldehyde

The compound was obtained from (3Z,6Z)-nona-3,6-dien-1-ol (2.81 g, 20.1 mmol), vanillin (4-hydroxy-3-methoxybenzaldehyde) (3.05 g, 20.1 mmol), triphenylphosphine (7.89 g, 30.1 mmol) and DIAD (diisopropyl azodicarboxylate) (6.08 g, 30.1 mmol) according to the procedure of example 1 as a colorless oil (1.71 g, 31% yield).

¹H NMR (400 MHz, CDCl3, 298 K) δ (ppm)=9.88-9.85 (m, 1H), 7.48-7.41 (m, 2H), 6.99 (d, J=8.3 Hz, 1H), 5.63-5.28 (m, 4H), 4.12 (t, J=7.2 Hz, 2H), 3.94 (s, 3H), 2.90-2.76 (m, 2H), 2.74-2.58 (m, 2H), 2.08 (dquin, J=1.3, 7.4 Hz, 2H), 0.98 (t, J=7.5 Hz, 3H). ¹³C NMR (101 MHz, CDCl3, 298 K) δ (ppm)=190.9, 153.9, 149.8, 132.3, 131.6, 130.0, 126.7, 126.7, 124.0, 111.4, 109.3, 68.4, 56.0, 27.2, 25.7, 20.6, 14.2. MS (EI, 70 eV): 274 (2, [M]+·), 152 (29), 122 (27), 93 (55), 81 (100), 79 (45), 77 (26), 69 (31), 67 (58), 55 (41), 41 (40).

Odour description (1% solution in EtOH on paper blotter, 24 h): green, leaf aldehyde, powdery, vanilla.

### Example 35: 3-methoxy-4-(tridec-1-en-4-yloxy)benzaldehyde

The compound was obtained from tridec-1-en-4-ol (1.98 g, 15.0 mmol), vanillin (4-hydroxy-3-methoxybenzaldehyde) (1.52 g, 10.0 mmol), triphenylphosphine (3.93 g, 15.0 mmol) and DIAD (diisopropyl azodicarboxylate) (3.03 g, 15.0 mmol) according to the procedure of example 1 as a colorless oil (0.88 g, 26% yield).

¹H NMR (400 MHz, CDCl3, 298 K) δ (ppm)=9.86 (s, 1H), 7.46-7.41 (m, 2H), 6.99 (d, J=8.8 Hz, 1H), 5.87 (tdd, J=7.1, 10.1, 17.1 Hz, 1H), 5.19-5.07 (m, 2H), 4.43 (quin, J=6.0 Hz, 1H), 3.93 (s, 3H), 2.59-2.41 (m, 2H), 1.86-1.69 (m, 2H), 1.55-1.21 (m, 14H), 0.93-0.86 (m, 3H). ¹³C NMR (101 MHz, CDCl3, 298 K) δ (ppm)=190.8, 153.7, 150.6, 133.8, 129.9, 126.5, 117.8, 113.5, 109.9, 79.1, 56.1, 38.3, 33.7, 31.9, 29.5, 29.5, 29.5, 29.3, 25.3, 22.7, 14.1. MS (EI, 70 eV): 332 (1, [M]+·), 153 (13), 152 (100), 151 (24), 83 (4), 69 (6), 67 (5), 55 (11), 43 (10), 41 (13), 29 (4).

Odour description (1% solution in EtOH on paper blotter, 24 h): powdery, vanilla, aldehydic, green, metallic, mandarin.

### Example 36: 4-(4-(tridec-1-en-4-yloxy)phenyl)butan-2-one

The compound was obtained from tridec-1-en-4-ol (3.97 g, 20.0 mmol), raspberry ketone (4-(4-hydroxyphenyl)butan-2-one) (3.28 g, 20.0 mmol), triphenylphosphine (7.87 g, 30.0 mmol) and DIAD (diisopropyl azodicarboxylate) (6.07 g, 30.0 mmol) according to the procedure of example 1 as a colorless oil (2.11 g, 29% yield).

¹H NMR (400 MHz, CDCl3, 298 K) δ (ppm)=7.14-7.06 (m, 2H), 6.84 (d, J=8.6 Hz, 2H), 5.94-5.79 (m, 1H), 5.19-5.05 (m, 2H), 4.24 (quin, J=5.9 Hz, 1H), 2.89-2.81 (m, 2H), 2.79-2.71 (m, 2H), 2.50-2.35 (m, 2H), 2.16 (s, 3H), 1.73-1.59 (m, 2H), 1.54-1.20 (m, 14H), 0.96-0.86 (m, 3H). ¹³C NMR (101 MHz, CDCl3, 298 K) δ (ppm)=208.1, 156.8, 134.3, 133.0, 129.2, 117.3, 116.1, 77.5, 45.5, 38.2, 33.7, 31.9, 30.1, 29.6, 29.6, 29.6, 29.3, 28.9, 25.4, 22.7, 14.1. MS (EI, 70 eV): 344 (4, [M]+·), 165 (11), 164 (97), 149 (9), 121 (16), 107 (100), 94 (32), 69 (9), 55 (21), 43 (43), 41 (22).

Odour description (1% solution in EtOH on paper blotter, 24 h): fruity, juicy, raspberry, aldehydic, green, metallic.

### Example 37: 2-ethoxy-4-(methoxymethyl)-1-(tridec-1-en-4-yloxy)benzene

The compound was obtained from tridec-1-en-4-ol (1.98 g, 10.0 mmol), 2-ethoxy-4-(methoxymethyl)phenol (1.82 g, 10.0 mmol), triphenylphosphine (3.93 g, 15.0 mmol) and DIAD (diisopropyl azodicarboxylate) (3.03 g, 15.0 mmol) according to the procedure of example 1 as a colorless oil (0.39 g, 2.7% yield).

¹H NMR (400 MHz, CDCl3, 298 K) δ (ppm)=6.94-6.88 (m, 2H), 6.87-6.82 (m, 1H), 5.92 (tdd, J=7.0, 10.1, 17.2 Hz, 1H), 5.17-5.04 (m, 2H), 4.39 (s, 2H), 4.22 (quin, J=5.9 Hz, 1H), 4.09 (q, J=7.1 Hz, 2H), 3.40 (s, 3H), 2.52-2.35 (m, 2H), 1.78-1.59 (m, 2H), 1.57-1.22 (m, 17H), 0.95-0.85 (m, 3H). ¹³C NMR (101 MHz, CDCl3, 298 K) δ (ppm)=150.4, 147.7, 134.7, 131.9, 120.5, 118.1, 117.0, 113.9, 80.1, 74.7, 64.5, 58.0, 38.4, 33.8, 31.9, 29.7, 29.6, 29.6, 29.3, 25.3, 22.7, 15.0, 14.1. MS (EI, 70 eV): 362 (4, [M]+), 321 (0), 182 (100), 181 (7). Odour description (1% solution in EtOH on paper blotter, 24 h): citrus, mandarin, green.

### Example 38: 3-methoxy-4-(tetradec-1-en-4-yloxy)benzaldehyde

The compound was obtained from tetradec-1-en-4-ol (2.12 g, 10.0 mmol), 4-hydroxy-3-methoxybenzaldehyde (1.52 g, 10.0 mmol), triphenylphosphine (3.93 g, 15.0 mmol) and DIAD (diisopropyl azodicarboxylate) (3.03 g, 15.0 mmol) according to the procedure of example 1 as a colorless oil (1.05 g, 90% purity, 27% yield).

¹H NMR (400 MHz, CDCl3, 298 K) δ (ppm)=9.85 (s, 1H), 7.45-7.41 (m, 2H), 6.99 (d, J=9.0 Hz, 1H), 5.93-5.79 (m, 1H), 5.19-5.07 (m, 2H), 4.42 (quin, J=6.0 Hz, 1H), 3.92 (s, 3H), 2.58-2.41 (m, 2H), 1.85-1.66 (m, 2H), 1.54-1.22 (m, 16H), 0.92-0.86 (m, 3H). ¹³C NMR (101 MHz, CDCl3, 298 K) δ (ppm)=190.8, 153.7, 150.6, 133.7, 129.9, 126.5, 117.8, 113.5, 109.9, 79.1, 56.0, 38.3, 33.7, 31.9, 31.9, 29.6, 29.5, 29.3, 25.3, 22.7, 14.1. MS (EI, 70 eV): 346 (2, [M]+·), 194 (1), 152 (100).

Odour description (1% solution in EtOH on paper blotter, 24 h): powdery, vanilla, creamy, aldehydic, mandarin, madeleine.

### Example 39: 4-(4-(pentadec-3-en-6-yloxy)phenyl)butan-2-one

### a) 4-(4-(pentadec-3-yn-6-yloxy)phenyl)butan-2-one

The compound was obtained from pentadec-3-yn-6-ol (1.04 g, 4.63 mmol), 4-(4-hydroxyphenyl)butan-2-one (0.761 g, 4.63 mmol), triphenylphosphine (1.824 g, 6.95 mmol) and DIAD (diisopropyl azodicarboxylate) (1.41 g, 6.95 mmol) according to the procedure of example 1 as a colorless oil (0.100 g, 5.8% yield) which was used without further purification. MS (EI, 70 eV): 370 (6, [M]+·), 303 (2), 245 (4), 164 (100), 107 (89), 43 (41).

### b) 4-(4-(pentadec-3-en-6-yloxy)phenyl)butan-2-one

A flask was charged with Lindlar catalyst (Palladium on calcium carbonate, poisoned with lead) (27 mg), toluene (7 mL), pyridine (0.7 mL) and 4-(4-(pentadec-3-yn-6-yloxy)phenyl)butan-2-one (0.270 g, 0.729 mmol) and the flask purged with nitrogen followed by hydrogen. The mixture was allowed to stir under hydrogen for 1.5 h at room temperature after which it was flushed with nitrogen and filtered through a plug of celite, rinsing with MTBE (methyl tert-butyl ether). The solvent was evaporated and the resulting material purified by chromatography on silica gel, eluting with a gradient of ethyl acetate in heptane to give 4-(4-(pentadec-3-en-6-yloxy)phenyl)butan-2-one (0.151 g, 56% yield).

¹H NMR (400 MHz, CDCl3, 298 K) δ (ppm)=7.12-7.06 (m, 2H), 6.86-6.81 (m, 2H), 5.64-5.34 (m, 2H), 4.29-4.14 (m, 1H), 2.91-2.80 (m, 2H), 2.80-2.70 (m, 2H), 2.48-2.32 (m, 2H), 2.15 (s, 3H), 2.07 (dqd, J=1.3, 7.5, 14.9 Hz, 2H), 1.70-1.59 (m, 2H), 1.54-1.21 (m, 14H), 1.03-0.94 (m, 3H), 0.93-0.86 (m, 3H). ¹³C NMR (101 MHz, CDCl3, 298 K) δ (ppm)=208.2, 156.8, 134.0, 132.9, 129.2, 124.0, 116.0, 77.9, 45.5, 33.8, 31.9, 31.5, 30.1, 29.7, 29.6, 29.5, 29.3, 29.3, 28.9, 25.5, 22.7, 20.8, 14.2, 14.1. MS (EI, 70 eV): 372 (5, [M]+·), 303 (5), 245 (11), 164 (100), 149 (10), 121 (17), 107 (83), 69 (36).

Odour description (1% solution in EtOH on paper blotter, 24 h): raspberry, mandarin, citrus, aldehydic, fresh.

### Example 40: ethyl 2-(2-ethoxy-4-formylphenoxy)hept-4-enoate

The compound was obtained from 3-ethoxy-4-hydroxybenzaldehyde (2.5 g, 15.0 mmol, 1.0 equiv) and ethyl 2-hydroxyhept-4-enoate (2.9 g, 16.6 mmol, 1.1 equiv) according to the procedure of example 1 as colorless liquid (6.6 mmol, 2.1 g, 44% yield).

¹H NMR (400 MHz, CDCl₃) δ 9.75 (s, 1H), 7.39-7.24 (m, 2H), 6.94-6.74 (m, 1H), 5.69-5.33 (m, 2H), 4.80-4.59 (m, 1H), 4.21-3.96 (m, 4H), 2.77-2.55 (m, 2H), 2.09-1.82 (m, 2H), 1.37 (t, J=6.9 Hz, 3H), 1.17 (t, J=7.1 Hz, 3H), 0.88 (t, J=7.5 Hz, 3H) ppm. ¹³C NMR (101 MHz, CDCl₃) δ 190.9 (d), 170.5 (s), 153.1 (s), 149.8 (s), 136.5 (d), 131.3 (s), 125.8 (d), 122.3 (d), 115.4 (d), 112.1 (d), 78.1 (d), 64.8 (t), 61.2 (t), 36.1 (t), 25.6 (t), 14.6 (q), 14.2 (q), 13.6 (q) ppm. GC/MS (EI): m/z (%): 320 (27) [M⁺], 247 (6), 219 (2), 166 (100), 138 (44), 109 (50), 55 (25).

Odor description (1% in alc, 24 h on blotter): powdery (vanilla)

### Example 41: 3-ethoxy-4-(((3Z,8Z)-undeca-3,8-dien-6-yl)oxy)benzaldehyde

### a) (3Z,8Z)-undeca-3,8-dien-6-ol

1,2-Butadiene (70 g, 1.3 mol, 3 equiv.) was dissolved in THF (400 mL). A solution of butyllithium (2.5M in hexane, 400 mL, 1 mol, 2.3 equiv.) was added dropwise at 10° C. under stirring over a period of 40 min, upon which the temperature rose to 25° C. and a clear yellow solution formed. Stirring was continued for 22 hours at RT yielding a beige suspension. Epichlorohydrine (40 g, 0.43 mol, 1 equiv.) was added dropwise during 10 min and the resulting mixture was refluxed for 23 h. The resulting clear, yellow solution was poured on cold 1N aq. HCl-solution (300 mL). Extraction with MTBE, washing with water and brine, drying over MgSO₄ and concentration i. RV. yielded a clear, yellow liquid (55.6 g, 78%). The crude product was distilled over a 15 cm Vigreux column (0.05 mbar/58° C.) to yield undeca-3,8-diyn-6-ol (31.9 g, 44%). The compound (6.5 g) was dissolved in hexane (100 mL) and Lindlar catalyst was added (0.20 g). Hydrogenation was effected under 1 bar H₂ at RT for 2 h. The mixture was filtered and dried to yield (3Z,8Z)-undeca-3,8-dien-6-ol (6.5 g, 99%).

¹H-NMR (400 MHz, CDCl₃): 5.53-5.61 (m, 2H), 5.40 (dtt, J=10.8, 7.5, 7.5, 1.6, 1.6 Hz, 2H), 3.63-3.68 (m, 1H), 2.24-2.28 (m, 4H), 2.05-2.13 (m, 4H), 1.78 (d, J=3.7 Hz, 1H), 0.98 (t, J=7.5 Hz, 6H). ¹³C-NMR (101 MHz, CDCl₃): 134.9 (2s), 124.5 (2s), 71.2 (s), 34.5 (2s), 20.7 (2s), 14.2 (2s). MS (EI, 70 eV): 150(2; [M-H₂O]⁺), 99(26), 81(97), 70(60), 69(28), 55(100), 41(64), 29(20).

### b) 3-ethoxy-4-(((3Z,8Z)-undeca-3,8-dien-6-yl)oxy)benzaldehyde

The compound was obtained from ethylvanillin (4.0 g), (3Z,8Z)-undeca-3,8-dien-6-ol (4.0 g), triphenyl phosphine (8.1 g) and diisopropyl azodicarboxylate (6.2 g) according to the procedure of example 1 as a clear yellow liquid (1.9 g, 25%).

¹H-NMR (400 MHz, CDCl3): 9.83 (s, 1H), 7.35-7.44 (m, 2H), 6.98 (d, J=8.8 Hz, 1H), 5.33-5.61 (m, 4H), 4.39 (quin, J=6.0 Hz, 1H), 4.12 (q, J=7.0 Hz, 2H), 2.30-2.54 (m, 4H), 1.98-2.13 (m, 4H), 1.44 (t, J=7.0 Hz, 3H), 0.96 (t, J=7.5 Hz, 6H). ¹³C-NMR (101 MHz, CDCl₃): 190.9 (s), 153.9 (s), 150.1 (s), 134.5 (2s), 130.2 (s), 126.2 (s), 123.6 (2s), 114.6 (s), 111.7 (s), 79.3 (s), 64.6 (s), 31.3 (2s), 20.8 (2s), 14.7 (s), 14.1 (2s). MS (EI, 70 eV): 316(4; M⁺), 247(2), 166(41), 151(17), 137(56), 121(28), 109(65), 95(97), 81(100), 69(85), 55(39), 41(84), 29(22).

Odour description (1% solution in EtOH on paper blotter, 24 h): vanilla, powdery.

### Example 42: 3-ethoxy-4-((4-methylnon-3-en-1-yl)oxy)benzaldehyde

To a solution of 3-ethoxy-4-hydroxybenzaldehyde (1.57 g, 9.45 mmol, 1.0 equiv) and potassium carbonate (1.97 g, 14.18 mmol) in DMF (15 mL) was added 1-bromo-4-methylnon-3-ene (2.69 g, 12.3 mmol, 1.4 equiv, prepared according to a procedure by Zarbin, P. H. G.; et al. Journal of Chemical Ecology 2012, 38, 825 using 1-cyclopropylethan-1-one and pentylmagnesium bromide) dropwise at r.t. and then stirred at 90° C. for 16 hours under argon atmosphere. The reaction conversion was monitored by TLC and GC. After cooling down the reaction solution to r.t., water (10 mL) and MTBE (10 mL) was added. The mixture was extracted with MTBE (2×10 mL), the organic layers were combined and washed with water (3×10 mL), and dried with MgSO₄ and the solvent was removed to give yellow oil. It was then purified by silica gel column chromatography (hexane:MTBE=94:6) to get 3-ethoxy-4-((4-methylnon-3-en-1-yl)oxy)benzaldehyde (0.72 g, 25% yield, E/Z 84:16) as light yellow liquid. ¹H NMR (400 MHz, CDCl₃, mixture of E/Z isomers): δ 9.83 (s, 1H), 7.42 (d, J=8.1 Hz, 1H), 7.40 (s, 1H), 6.97 (d, J=7.9 Hz, 1H), 5.23-5.17 (m, 1H), 4.18-4.03 (m, 4H), 2.58 (q, J=7.3 Hz, 2H), 2.09-1.95 (m, 2H), 1.76-1.64 (m, 3H), 1.50-1.20 (m, 9H), 0.92-0.84 (m, 3H) ppm. ¹³C NMR (101 MHz, CDCl₃, mixture of E/Z isomers): δ 190.9, 154.4, 154.3, 149.1, 149.1, 139.2, 139.0, 129.9, 129.9, 126.6, 119.0, 118.3, 111.7, 110.8, 110.8, 68.8, 68.6, 64.5, 39.7, 31.9, 31.8, 31.5, 27.9, 27.7, 27.7, 27.5, 23.5, 22.6, 22.5, 16.1, 14.7, 14.0 ppm.

Odour description (1% solution in EtOH on paper blotter, 24 h): powdery, vanilla, sweet, sugary.

### Example 43: 3-ethoxy-4-(undec-2-en-5-yloxy)benzaldehyde

The compound was obtained from 3-ethoxy-4-hydroxybenzaldehyde (1.00 g, 6.02 mmol, 1.0 equiv) and undec-2-en-5-ol (1.13 g, 6.62 mmol, 1.1 equiv) according to according to the procedure of example 1 as light yellow liquid (0.30 g, 16% yield, E/Z 73:27).

¹H NMR (400 MHz, CDCl₃, mixture of E/Z isomers): δ 9.83 (s, 1H), 7.42-7.39 (m, 2H), 7.04-6.89 (m, 1H), 5.64-5.40 (m, 2H), 4.43-4.26 (m, 1H), 4.13 (q, J=6.9 Hz, 2H), 2.48-2.33 (m, 2H), 1.77-1.62 (m, 5H), 1.51-1.25 (m, 11H), 0.93-0.81 (m, 3H) ppm. ¹³C NMR (101 MHz, CDCl₃, mixture of E/Z isomers): δ 190.8, 154.2, 154.1, 150.0, 130.0, 128.2, 126.6, 126.2, 126.2, 125.2, 114.6, 114.4, 111.8, 111.7, 79.9, 79.6, 64.6, 64.6, 37.0, 33.7, 33.6, 31.7, 31.3, 29.2, 25.3, 25.2, 22.5, 18.0, 14.7, 14.0, 13.0 ppm.

Odour description (1% solution in EtOH on paper blotter, 24 h): powdery, creamy, vanilla.

### Example 44: 4-(dodec-3-en-6-yloxy)-3-ethoxybenzaldehyde

The compound was obtained from 3-ethoxy-4-hydroxybenzaldehyde (1.07 g, 6.41 mmol, 1.0 equiv) and dodec-3-en-6-ol (1.30 g, 7.05 mmol, 1.1 equiv) according to the procedure of example 1 as light yellow liquid (0.50 g, 23% yield, E/Z 85:15).

¹H NMR (400 MHz, CDCl₃, mixture of E/Z isomers): δ 9.83 (s, 1H), 7.44-7.37 (m, 2H), 6.97 (d, J=8.8 Hz, 1H), 5.68-5.27 (m, 2H), 4.40-4.28 (m, 1H), 4.13 (q, J=7.1 Hz, 2H), 2.50-2.32 (m, 2H), 2.08-1.97 (m, 2H), 1.76-1.64 (m, 2H), 1.50-1.23 (m, 11H), 0.99-0.93 (m, 3H), 0.90-0.85 (m, 3H) ppm. ¹³C NMR (101 MHz, CDCl₃, mixture of E/Z isomers): δ 190.9, 154.3, 154.1, 150.0, 135.4, 134.3, 130.0, 130.0, 126.2, 123.9, 123.6, 114.6, 114.3, 111.8, 111.7, 79.9, 79.6, 64.6, 64.6, 37.0, 33.7, 33.6, 31.7, 31.6, 29.2, 25.6, 25.3, 25.2, 22.5, 20.7, 14.7, 14.7, 14.1, 14.0, 13.7 ppm.

Odour description (1% solution in EtOH on paper blotter, 24 h): woody, cedarwood, vanilla.

### Example 45: methyl (Z)-12-(2-methoxyphenoxy)octadec-9-enoate

The compound was obtained from 2-methoxyphenol (2.00 g, 16.1 mmol, 1.0 equiv) and methyl (Z)-12-hydroxyoctadec-9-enoate (5.54 g, 17.7 mmol, 1.1 equiv) according to the procedure of example 1 as pink liquid (2.65 g, 39% yield).

¹H NMR (400 MHz, CDCl₃): δ 6.97-6.83 (m, 4H), 5.53-5.40 (m, 2H), 4.27-4.15 (m, 1H), 3.85 (s, 3H), 3.67 (s, 3H), 2.51-2.35 (m, 2H), 2.31 (t, J=7.6 Hz, 2H), 2.08-1.97 (m, 2H), 1.62 (s, 4H), 1.56-1.46 (m, 1H), 1.43-1.22 (m, 15H), 0.92-0.84 (m, 3H) ppm. ¹³C NMR (101 MHz, CDCl₃): δ 174.2, 150.6, 147.9, 132.1, 124.9, 121.3, 120.8, 116.3, 112.3, 79.6, 55.9, 51.4, 34.1, 33.9, 31.8, 31.8, 29.5, 29.3, 29.1, 29.1, 29.1, 27.4, 25.4, 24.9, 22.6, 14.1 ppm.

Odour description (1% solution in EtOH on paper blotter, 24 h): phenolic, medicinal, guaiacol, vanilla absolute, peru balsam.

### Example 46: 1-(dec-1-en-4-yloxy)-2-ethoxy-4-methylbenzene and 2-(dec-1-en-4-yloxy)-1-ethoxy-4-methylbenzene

The compound was obtained from a 1:1 mixture of 2-ethoxy-4-methylphenol and 2-ethoxy-5-methylphenol (1.33 g, 8.73 mmol, 1.0 equiv, prepared according to EP0179532) and dec-1-en-4-ol (1.50 g, 9.60 mmol, 1.1 equiv) according to the procedure of example 1 as a colorless liquid (0.9 g, 36% yield, mixture of regioisomers 55:45).

¹H NMR (400 MHz, CDCl₃, mixture of regioisomers): δ 6.88-6.78 (m, 1H), 6.77-6.65 (m, 2H), 6.00-5.85 (m, 1H), 5.18-5.04 (m, 2H), 4.27-4.12 (m, 1H), 4.05 (dd, J=6.2, 7.0 Hz, 2H), 2.54-2.35 (m, 2H), 2.29 (d, J=6.8 Hz, 3H), 1.79-1.59 (m, 2H), 1.56-1.39 (m, 5H), 1.36-1.24 (m, 6H), 0.90 (br d, J=1.2 Hz, 3H) ppm. ¹³C NMR (101 MHz, CDCl₃, mixture of regioisomers): δ 150.2, 148.1, 148.0, 145.8, 134.9, 134.8, 131.7, 130.6, 122.1, 121.1, 119.3, 118.8, 116.9, 116.8, 115.2, 114.7, 80.2, 79.8, 64.9, 64.4, 38.4, 33.8, 33.8, 31.8, 31.8, 29.4, 29.4, 25.3, 25.3, 22.6, 21.0, 20.8, 15.0, 15.0, 14.1 ppm.

Odour description (1% solution in EtOH on paper blotter, 24 h): medicinal, phenolic, vanilla.

### Example 47: 3-ethoxy-4-((4-methyl-6-phenylhex-3-en-1-yl)oxy)benzaldehyde

### a) (6-bromo-3-methylhex-3-en-1-yl)benzene

Magnesium powder (6.94 g, 285 mmol, 1.2 equiv) was added to a 500 mL-sulfonation flask and overlayed with THF (20 mL). Then a solution of (2-bromoethyl)benzene (52.8 g, 285 mmol, 1.2 equiv) in THF (100 mL) was added dropwise, after having started the Grignard reaction using a catalytic amount of iodine and by heating to reflux with a heatgun. The reaction mixture was refluxed for 1 hour. Then, a solution of 1-cyclopropylmethyl ketone (20.0 g, 238 mmol, 1.0 equiv) in THF (100 mL) was added dropwise to the Grignard reagent. After stirring for 1 hour at room temperature, a solution of 54 mL of 63% sulfuric acid (in water) was added dropwise at 0° C. and the reaction mixture was stirred for 18 hours at room temperature. After quenching with water (200 mL), the reaction mixture was extracted with MTBE (2×250 mL). The combined organic layers were washed with 2M NaOH (250 mL), water (250 mL), brine (250 mL), dried over MgSO4 and the solvent was removed under reduced pressure. The crude product was distilled over a 3 cm Vigreux column under high vacuum (0.04 mbar) to give (6-bromo-3-methylhex-3-en-1-yl)benzene (40.6 g, 67 yield, E/Z 80:20) as a colorless liquid.

¹³C NMR (101 MHz, CDCl₃, mixture of E/Z isomers): δ 142.0, 137.9, 137.5, 128.4, 128.3, 128.3, 128.2, 125.9, 125.7, 122.3, 121.4, 41.4, 34.5, 34.1, 34.1, 32.7, 32.6, 31.6, 31.5, 23.3, 16.4 ppm.

### b) 3-ethoxy-4-((4-methyl-6-phenylhex-3-en-1-yl)oxy)benzaldehyde

The compound was obtained from 3-ethoxy-4-hydroxybenzaldehyde (6.56 g, 39.5 mmol, 1.0 equiv) and (6-bromo-3-methylhex-3-en-1-yl)benzene (10.0 g, 39.5 mmol, 1.0 equiv) according to the procedure of example 42 as a slightly yellow liquid (7.40 g, 55% yield, E/Z 82:18).

¹H NMR (400 MHz, CDCl₃, mixture of E/Z isomers): δ 9.83 (s, 0.82H), 9.82 (s, 0.18H), 7.46-7.36 (m, 2H), 7.29-7.20 (m, 2H), 7.17 (s, 3H), 6.93 (d, J=8.1 Hz, 0.82H), 6.86 (d, J=8.1 Hz, 0.18H), 5.29-5.12 (m, 1H), 4.21-4.07 (m, 2H), 4.00 (t, J=7.1 Hz, 1.64H), 3.85 (t, J=7.2 Hz, 0.36H), 2.79-2.66 (m, 2H), 2.62-2.51 (m, 1.64H), 2.45 (dq, J=1.0, 7.3 Hz, 0.36H), 2.41-2.35 (m, 0.36H), 2.35-2.27 (m, 1.64H), 1.78 (d, J=1.2 Hz, 0.54H), 1.73 (s, 2.46H), 1.52-1.41 (m, 3H) ppm. ¹³C NMR (101 MHz, CDCl₃, mixture of E/Z isomers): δ 190.9, 154.3, 154.3 (Z), 149.1, 149.1 (Z), 142.1, 142.0 (Z), 137.9, 137.6 (Z), 129.9, 129.9, 128.4, 128.4, 128.2, 128.2, 126.5, 126.5, 125.8, 125.7, 120.3 (Z), 119.4, 111.8 (Z), 111.7, 110.8, 68.5, 68.5, 64.5, 41.6, 34.5, 34.2 (Z), 34.0 (Z), 27.8, 27.7 (Z), 23.5 (Z), 16.3, 14.7, 14.6 (Z) ppm. GC/MS (EI) major E isomer: m/z (%): 338 (0, [M]⁺·), 132 (5), 131 (49), 117 (10), 105 (6), 92 (8), 91 (100), 81 (7), 69 (6), 67 (7), 65 (6). GC/MS (EI) minor Z isomer: m/z (%): 132 (5), 131 (44), 117 (11), 105 (7), 92 (8), 91 (100), 81 (7), 69 (6), 67 (6), 65 (6).

Odour description (1% solution in EtOH on paper blotter, 24 h): sweet, vanilla, ice cream, powdery, fruity, raspberry, sorbet.

### Example 48: (E)-3-ethoxy-4-((4-phenylpent-3-en-1-yl)oxy)benzaldehyde

The compound was obtained from 3-ethoxy-4-hydroxybenzaldehyde (1.00 g, 6.02 mmol, 1.0 equiv) and (5-bromopent-2-en-2-yl)benzene (1.36 g, 6.02 mmol, 1.0 equiv, prepared according to Brucher, O. et al. ACS Catalysis 2011, 1, 1448) according to the procedure of example 42 as a slightly yellow liquid (0.44 g, 26% yield, E/Z 82:18).

¹H NMR (400 MHz, CDCl₃, mixture of E/Z isomers): δ 9.84 (s, 1H), 7.49-7.36 (m, 4H), 7.35-7.28 (m, 2H), 7.27-7.19 (m, 1H), 6.99 (d, J=8.1 Hz, 1H), 5.85 (qt, J=1.3, 7.3 Hz, 1H), 4.19 (t, J=7.1 Hz, 2H), 4.15 (q, J=7.0 Hz, 2H), 2.85-2.71 (m, 2H), 2.11 (d, J=1.2 Hz, 3H), 1.47 (t, J=7.0 Hz, 3H) ppm. ¹³C NMR (101 MHz, CDCl₃, mixture of E/Z isomers): δ 190.9, 154.3, 149.2, 143.5, 137.9, 130.1, 128.2, 126.9, 126.5, 125.7, 122.5, 111.9, 110.9, 68.3, 64.5, 28.7, 16.1, 14.7 ppm.

Odour description (1% solution in EtOH on paper blotter, 24 h): sweet, powdery, vanilla, ice cream.

### Example 49: 3-ethoxy-4-((4-(4-methoxyphenyl)pent-3-en-1-yl)oxy)benzaldehyde

The compound was obtained from 3-ethoxy-4-hydroxybenzaldehyde (1.89 g, 11.4 mmol, 1.0 equiv) and 1-(5-bromopent-2-en-2-yl)-4-methoxybenzene (2.90 g, 11.4 mmol, 1.0 equiv, prepared according to Bruecher, O. et al., Tetrahedron 2012, 6, 6968) according to the procedure of example 42 as a colorless liquid (1.17 g, 30% yield, E/Z 99:1).

¹H NMR (400 MHz, CDCl₃, major E isomer): δ 9.84 (s, 1H), 7.42 (d, J=2.9 Hz, 2H), 7.34 (d, J=9.0 Hz, 2H), 6.99 (d, J=8.1 Hz, 1H), 6.91-6.79 (m, 2H), 5.79 (qt, J=1.2, 7.3 Hz, 1H), 4.16 (m, 4H), 3.81 (s, 3H), 2.78 (q, J=7.1 Hz, 2H), 2.09 (d, J=1.2 Hz, 3H), 1.48 (t, J=7.0 Hz, 3H) ppm. ¹³C NMR (101 MHz, CDCl₃, major E isomer): δ 190.9, 158.6, 154.3, 149.1, 137.1, 135.9, 130.0, 126.6, 126.5, 120.8, 113.5, 111.8, 110.8, 68.4, 64.5, 55.2, 28.6, 16.0, 14.6 ppm. Odour description (1% solution in EtOH on paper blotter, 24 h): sweet, powdery, vanilla, ice cream, soft anisic, heliotropine, anethiol.

### Example 50: 3-ethoxy-4-((4-methyl-5-phenylpent-3-en-1-yl)oxy)benzaldehyde

The compound was obtained from 3-ethoxy-4-hydroxybenzaldehyde (2.00 g, 12.0 mmol, 1.0 equiv) and (5-bromo-2-methylpent-2-en-1-yl)benzene (2.90 g, 12.0 mmol, 1.0 equiv, prepared according to Krafft, M. E. Tetrahedron Lett. 1989, 3, 539) according to the procedure of example 42 as a colorless liquid (0.72 g, 18% yield, E/Z 80:20).

¹H NMR (400 MHz, CDCl₃, mixture of E/Z isomers): δ 9.83 (s, 1H), 7.40 (s, 2H), 7.26 (s, 2H), 7.17 (s, 3H), 6.97 (d, J=8.1 Hz, 1H), 5.41 (dt, J=1.2, 7.3 Hz, 0.2H), 5.34 (qt, J=1.2, 7.3 Hz, 0.8H), 4.24-4.00 (m, 4H), 3.54-3.38 (m, 0.4H), 3.33 (s, 1.6H), 2.73 (dq, J=1.0, 7.2 Hz, 0.4H), 2.62 (q, J=7.1 Hz, 1.6H), 1.65-1.59 (m, 3H), 1.50-1.39 (m, 3H) ppm. ¹³C NMR (101 MHz, CDCl₃, mixture of E/Z isomers): δ 190.9, 154.3, 149.1, 149.1, 139.9, 139.7, 138.0, 137.3, 130.0, 129.9, 128.8, 128.4, 128.4, 128.2, 126.6, 126.0, 126.0, 120.9, 120.9, 111.8, 111.7, 110.8, 68.7, 68.5, 64.5, 46.2, 37.9, 28.1, 28.0, 23.5, 16.1, 14.7, 14.6 ppm.

Odour description (1% solution in EtOH on paper blotter, 24 h): powdery, vanilla, ice cream.

### Example 51: (E)-3-ethoxy-4-((4-(naphthalen-2-yl)pent-3-en-1-yl)oxy)benzaldehyde

The compound was obtained from 3-ethoxy-4-hydroxybenzaldehyde (12.4 g, 74.6 mmol, 1.0 equiv) and (E)-2-(5-bromopent-2-en-2-yl)naphthalene (27.4 g, 74.6 mmol, 1.0 equiv, 75% purity, prepared according to Liu, X. et al., J. Am. Chem. Soc. 2018, 140, 4782) according to the procedure of example 42 as a colorless solid (12.2 g, 45% yield), after purification by fractional distillation under high vacuum (0.06 mbar).

¹H NMR (400 MHz, CDCl₃): δ 9.86 (s, 1H), 7.87-7.78 (m, 4H), 7.61 (dd, J=1.8, 8.7 Hz, 1H), 7.51-7.40 (m, 4H), 7.00 (d, J=8.6 Hz, 1H), 6.07-6.02 (m, 1H), 4.22 (t, J=7.1 Hz, 2H), 4.16 (q, J=7.1 Hz, 2H), 2.97-2.78 (m, 2H), 2.24 (d, J=1.2 Hz, 3H), 1.49 (t, J=7.0 Hz, 3H) ppm. ¹³C NMR (101 MHz, CDCl₃): δ 190.8, 154.2, 149.1, 140.5, 137.5, 133.3, 132.4, 130.0, 127.9, 127.6, 127.4, 126.4, 126.0, 125.5, 124.2, 124.1, 123.1, 111.8, 110.8, 68.2, 64.4, 28.8, 16.0, 14.6 ppm. GC/MS (EI): m/z (%): 360 (7, [M]⁺·), 195 (100), 181 (21), 180 (24), 179 (45), 178 (32), 167 (23), 166 (39), 165 (76), 153 (32), 152 (21).

Odour description (1% solution in EtOH on paper blotter, 24 h): powdery, vanilla, ice cream, floral, neroli, orange flower, baby cologne, madeleine, fougassette.

### Example 52: 4-(4-((4-methyl-6-phenylhex-3-en-1-yl)oxy)phenyl)butan-2-one

The compound was obtained from 4-(4-hydroxyphenyl)butan-2-one (6.49 g, 39.5 mmol, 1.0 equiv) and (6-bromo-3-methylhex-3-en-1-yl)benzene (10.0 g, 39.5 mmol, 1.0 equiv) according to the procedure of example 42 as a slightly yellow liquid (4.69 g, 35% yield, E/Z 80:20).

¹H NMR (400 MHz, CDCl₃, mixture of E/Z isomers): δ 7.31-7.22 (m, 2H), 7.22-7.13 (m, 3H), 7.12-7.03 (m, 2H), 6.86-6.69 (m, 2H), 5.30-5.12 (m, 1H), 3.85 (t, J=7.1 Hz, 1.6H), 3.73 (t, J=7.0 Hz, 0.4H), 2.90-2.78 (m, 2H), 2.76-2.66 (m, 4H), 2.46 (q, J=6.8 Hz, 1.6H), 2.40-2.26 (m, 2.4H), 2.12 (s, 3H), 1.76 (d, J=1.5 Hz, 0.6H), 11.70 (s, 2.4H). ¹³C NMR (101 MHz, CDCl₃, mixture of E/Z isomers): δ 208.1, 157.3, 157.3 (Z), 142.2, 142.1 (Z), 137.3, 137.1 (Z), 132.9, 132.8 (Z), 129.1, 129.1 (Z), 128.4 (Z), 128.4, 128.2 (Z), 128.2, 125.8 (Z), 125.6, 121.0 (Z), 120.0, 114.5, 114.5 (Z), 67.6 (Z), 67.5, 45.4, 41.6, 34.6, 34.3 (Z), 34.1 (Z), 30.1, 28.9, 28.1, 28.0 (Z), 23.4 (Z), 16.3 ppm. GC/MS (EI) major E isomer: m/z (%): 336 (1, [M]⁺·), 164 (12), 131 (44), 117 (10), 107 (8), 105 (7), 92 (8), 91 (100), 69 (6), 67 (7), 43 (12). GC/MS (EI) minor Z isomer: m/z (%): 336 (2, [M]⁺·), 164 (13), 131 (41), 117 (9), 107 (8), 105 (7), 92 (8), 91 (100), 69 (6), 67 (6), 43 (11).

Odour description (1% solution in EtOH on paper blotter, 24 h): sweet, crystal sugar, fruity, raspberry.

### Example 53: 4-(4-((4-(4-methoxyphenyl)pent-3-en-1-yl)oxy)phenyl)butan-2-one

The compound was obtained from 4-(4-hydroxyphenyl)butan-2-one (1.87 g, 11.4 mmol, 1.0 equiv) and 1-(5-bromopent-2-en-2-yl)-4-methoxybenzene (2.91 g, 11.4 mmol, 1.0 equiv, prepared according to Bruecher, O. et al., Tetrahedron 2012, 6, 6968) according to the procedure of example 42 as a slightly yellow liquid (0.52 g, 13% yield, E/Z 93:7).

¹H NMR (400 MHz, CDCl₃, major E isomer): δ 7.42-7.30 (m, 2H), 7.16-7.02 (m, 2H), 6.91-6.74 (m, 4H), 5.79 (t, J=7.1 Hz, 1H), 4.03 (t, J=7.0 Hz, 2H), 3.82 (s, 3H), 2.88-2.80 (m, 2H), 2.77-2.64 (m, 4H), 2.14 (s, 3H), 2.07 (d, J=1.2 Hz, 3H) ppm. ¹³C NMR (101 MHz, CDCl₃, major E isomer): δ 208.2, 158.6, 157.3, 136.5, 136.1, 133.0, 129.3, 129.2, 129.1, 126.6, 121.7, 114.6, 113.5, 67.4, 55.3, 45.4, 30.1, 29.0, 28.9, 16.0 ppm.

Odour description (1% solution in EtOH on paper blotter, 24 h): sweet powdery, anisic aldehyde, fruity, raspberry ketone.

### Example 54: 3-ethoxy-4-((6-(4-methoxyphenyl)-4-methylhex-3-en-1-yl)oxy)benzaldehyde

The compound was obtained from 3-ethoxy-4-hydroxybenzaldehyde (2.50 g, 15.0 mmol, 1.0 equiv) and 1-(6-chloro-3-methylhex-3-en-1-yl)-4-methoxybenzene (3.95 g, 16.5 mmol, 1.0 equiv, prepared according to Bruecher, O.; et al. Tetrahedron 2012, 68, 6968) according to the procedure of example 42 as a slightly yellow liquid (3.58 g, 65% yield, E/Z 80:20).

¹H NMR (400 MHz, CDCl₃, mixture of E/Z isomers): δ 9.84 (2s, 1H), 7.48-7.35 (m, 2H), 7.13-7.04 (m, 2H), 6.95 (d, J=8.3 Hz, 0.8H), 6.87 (d, J=8.1 Hz, 0.2H), 6.84-6.77 (m, 2H), 5.24 (dt, J=1.2, 7.3 Hz, 0.2H), 5.19 (qt, J=1.2, 7.3 Hz, 0.8H), 4.21-4.09 (m, 2H), 4.01 (t, J=7.2 Hz, 1.6H), 3.83 (t, J=7.2 Hz, 0.4H), 3.77 (s, 2.4H), 3.75 (s, 0.6H), 2.72-2.64 (m, 2H), 2.57 (q, J=7.1 Hz, 1.6H), 2.46 (dq, J=0.9, 7.3 Hz, 0.4H), 2.36 (pseudo t, J=7.7 Hz, 0.4H), 2.33-2.26 (m, 1.6H), 1.79 (d, J=1.2 Hz, 0.6H), 1.74 (s, 2.4H), 1.48 (t, J=7.0 Hz, 2.4H), 1.45 (t, J=7.1 Hz, 0.6H). ¹³C NMR (101 MHz, CDCl₃, mixture of E/Z isomers): δ 190.9, 157.7, 157.6, 154.3, 154.3, 149.1, 149.0, 137.9, 137.6, 134.1, 134.0, 129.9, 129.8, 129.3, 129.2, 126.5, 120.2, 119.3, 113.6, 113.5, 111.6, 111.6, 110.7, 68.5, 68.5, 64.4, 55.1, 41.8, 34.2, 33.5, 33.2, 27.8, 27.7, 23.4, 16.2, 14.6, 14.6 ppm. GC/MS (EI) major E isomer: m/z (%): 368 (0, [M]⁺·), 203 (3), 122 (9), 121 (100), 91 (3), 81 (2), 78 (3), 77 (3), 67 (3), 65 (1), 41 (1). GC/MS (EI) minor Z isomer: m/z (%): 368 (0, [M]⁺·), 203 (3), 137 (2), 122 (9), 121 (100), 91 (3), 81 (2), 78 (3), 77 (3), 67 (3), 41 (1).

Odour description (1% solution in EtOH on paper blotter, 24 h): powdery, vanilla.

### Example 55: 4-(4-((6-(4-methoxyphenyl)-4-methylhex-3-en-1-yl)oxy)phenyl)butan-2-one

The compound was obtained from 4-(4-hydroxyphenyl)butan-2-one (2.50 g, 15.2 mmol, 1.0 equiv) and 1-(6-chloro-3-methylhex-3-en-1-yl)-4-methoxybenzene (4.00 g, 16.7 mmol, 1.0 equiv, prepared according to Bruecher, O.; et al. Tetrahedron 2012, 68, 6968) according to the procedure of example 42 as colorless liquid (1.95 g, 35% yield, E/Z 80:20).

¹H NMR (400 MHz, CDCl₃, mixture of E/Z isomers): δ 7.17-7.01 (m, 4H), 6.89-6.74 (m, 4H), 5.34-5.15 (m, 1H), 3.88 (t, J=7.1 Hz, 1.6H), 3.79 (s, 2.4H), 3.77 (s, 0.6H), 3.74 (t, J=7.1 Hz, 0.4H), 2.90-2.81 (m, 2H), 2.79-2.64 (m, 4H), 2.54-2.45 (m, 1.6H), 2.42-2.26 (m, 2.4H), 2.15 (s, 3H), 1.79 (d, J=1.5 Hz, 0.6H), 1.72 (s, 2.4H) ppm. ¹³C NMR (101 MHz, CDCl₃, mixture of E/Z isomers): δ 208.1, 157.7, 157.6, 157.3, 137.3, 137.1, 134.2, 134.1, 132.8, 132.8, 129.3, 129.2, 129.1, 129.1, 120.9, 120.0, 114.5, 114.4, 113.6, 113.6, 67.5, 67.5, 55.1, 55.1, 45.4, 41.8, 34.2, 33.6, 33.3, 30.0, 28.9, 28.1, 28.0, 23.4, 16.3 ppm. GC/MS (EI) major E isomer: m/z (%): 366 (1, [M]+·), 202 (2), 122 (9), 121 (100), 119 (1), 107 (2), 91 (3), 78 (3), 77 (4), 67 (3), 43 (4). GC/MS (EI) minor Z isomer: m/z (%): 366 (1, [M]+·), 202 (2), 122 (9), 121 (100), 107 (2), 91 (4), 78 (3), 77 (3), 67 (3), 43 (5), 41 (1).

Odour description (1% solution in EtOH on paper blotter, 24 h): sweet, fruity, raspberry.

### Example 56: methyl 2-((4-methyl-6-phenylhex-3-en-1-yl)oxy)benzoate

The compound was obtained from methyl 2-hydroxybenzoate (1.20 g, 7.90 mmol, 1.0 equiv) and (6-bromo-3-methylhex-3-en-1-yl)benzene (2.00 g, 7.90 mmol, 1.0 equiv) according to the procedure of example 42 as a yellow liquid (1.01 g, 39% yield, E/Z 80:20).

¹H NMR (400 MHz, CDCl₃, mixture of E/Z isomers): δ 7.85-7.77 (m, 1H), 7.53-7.41 (m, 1H), 7.34-7.26 (m, 2H), 7.25-7.15 (m, 3H), 7.05-6.85 (m, 2H), 5.37-5.13 (m, 1H), 3.99 (t, J=7.1 Hz, 1.6H), 3.91 (s, 2.4H), 3.90 (s, 0.4H), 3.84 (t, J=7.1 Hz, 0.6H), 2.83-2.69 (m, 2H), 2.62-2.53 (m, 1.6H), 2.50-2.30 (m, 2.4H), 1.83-1.80 (m, 0.6H), 1.75 (s, 2.4H). ¹³C NMR (101 MHz, CDCl₃, mixture of E/Z isomers): δ 13C NMR (101 MHz, CDCl3, 298 K) Shift (ppm)=166.9, 158.4, 142.2, 137.3, 137.0, 133.2, 133.2, 131.5, 131.5, 128.4, 128.3, 128.2, 128.2, 125.8, 125.6, 120.8, 120.6, 120.1, 120.1, 119.9, 119.8, 113.4, 113.3, 68.6, 68.5, 51.9, 41.6, 34.5, 34.2, 34.0, 28.0, 27.9, 23.5, 16.3 ppm.

Odour description (1% solution in EtOH on paper blotter, 24 h): wintergreen, salicylate, slightly powdery, vanilla.

### Example 57: 1-(2-((4-methyl-6-phenylhex-3-en-1-yl)oxy)phenyl)propan-1-one

The compound was obtained from 1-(2-hydroxyphenyl)propan-1-one (1.19 g, 7.90 mmol, 1.0 equiv) and (6-bromo-3-methylhex-3-en-1-yl)benzene (2.00 g, 7.90 mmol, 1.0 equiv) according to the procedure of example 42 as a slightly yellow liquid (0.82 g, 32% yield, E/Z 75:25).

¹H NMR (400 MHz, CDCl₃, mixture of E/Z isomers): δ 7.73-7.64 (m, 1H), 7.45-7.35 (m, 1H), 7.30-7.22 (m, 2H), 7.20-7.12 (m, 3H), 6.97 (s, 1H), 6.91 (d, J=7.8 Hz, 0.75H), 6.83 (d, J=8.3 Hz, 0.25H), 5.30-5.12 (m, 1H), 3.99 (t, J=6.7 Hz, 1.5H), 3.80 (t, J=6.8 Hz, 0.5H), 3.04-2.92 (m, 2H), 2.77-2.66 (m, 2H), 2.58-2.48 (m, 1.5H), 2.41-2.26 (m, 2.5H), 1.78 (d, J=1.2 Hz, 0.75H), 1.71 (d, J=0.7 Hz, 2.25H), 1.15 (2×t, J=7.1 Hz and J=7.3 Hz, 3H). ¹³C NMR (101 MHz, CDCl₃, mixture of E/Z isomers): δ 203.6, 203.5, 157.9, 157.8, 142.0, 141.9, 137.4, 137.1, 133.1, 133.0, 130.2, 130.2, 128.4, 128.3, 128.3, 128.2, 125.9, 125.7, 121.1, 120.4, 120.4, 120.2, 112.2, 112.1, 68.1, 68.0, 41.6, 37.1, 34.5, 34.1, 34.0, 28.1, 27.8, 23.4, 16.3, 8.6 ppm.

Odour description (1% solution in EtOH on paper blotter, 24 h): wintergreen, salicylate, slightly powdery, vanilla.

### Example 58: 3-ethoxy-4-((4-methyl-7-phenylhept-3-en-1-yl)oxy)benzaldehyde

### a) (7-chloro-4-methylhept-4-en-1-yl)benzene

Magnesium powder (1.73 g, 71.3 mmol, 1.2 equiv) was added to a 100 ml-sulfonation flask and overlayed with THF (5 mL). Then a solution of (3-chloropropyl)benzene (11.0 g, 71.3 mmol, 1.2 equiv) in THF (30 mL) was added dropwise, after having started the Grignard reaction using a catalytic amount of iodine and by heating to reflux with a heatgun. The reaction mixture was stirred for 1 hour at room temperature. Then, a solution of 1-cyclopropylmethyl ketone (5.00 g, 59.4 mmol, 1.0 equiv) in THF (15 mL) was added dropwise to the Grignard reagent. After stirring for 3 hours at room temperature, a solution of 15 mL of 63% sulfuric acid (in water) was added dropwise at 3° C. and the reaction mixture was stirred for 18 hours at room temperature. After quenching with water (20 mL), the mixture was extracted with MTBE (30 mL). The organic layer was washed with water (30 mL), 2M NaOH (30 mL), water (30 mL), brine (30 mL), dried over MgSO4 and the solvent was removed under reduced pressure. The crude product was distilled using a Kugelrohr oven (130° C., 0.02 mbar) to give (7-chloro-4-methylhept-4-en-1-yl)benzene (11.1 g, 59% purity, 41% yield) as a colorless liquid, which was used in the next step without further purification.

### b) 3-ethoxy-4-((4-methyl-7-phenylhept-3-en-1-yl)oxy)benzaldehyde

The compound was obtained from 3-ethoxy-4-hydroxybenzaldehyde (2.50 g, 15.0 mmol, 1.0 equiv) and (7-chloro-4-methylhept-4-en-1-yl)benzene (3.35 g, 15.0 mmol, 1.0 equiv) according to the procedure of example 42 as a yellow liquid (1.39 g, 26% yield, E/Z 75:25).

¹H NMR (400 MHz, CDCl₃, mixture of E/Z isomers): δ 9.82 (2×s, 1H), 7.44-7.36 (m, 2H), 7.30-7.23 (m, 2H), 7.22-7.12 (m, 3H), 7.01-6.86 (m, 1H), 5.31-5.14 (m, 1H), 4.20-3.99 (m, 4H), 2.65-2.50 (m, 4H), 2.18-2.01 (m, 2H), 1.79-1.64 (m, 5H), 1.50-1.41 (m, 3H) ppm. ¹³C NMR (101 MHz, CDCl₃, mixture of E/Z isomers): δ 190.9, 154.3, 154.3, 149.1, 149.1, 142.5, 142.3, 138.4, 129.9, 129.9, 128.4, 128.4, 128.4, 128.3, 128.3, 128.2, 126.5, 125.7, 125.6, 119.7, 119.0, 111.7, 111.7, 110.8, 68.7, 68.6, 64.5, 39.2, 35.8, 35.4, 31.6, 29.7, 29.5, 27.9, 27.7, 23.4, 22.7, 16.1, 14.6 ppm.

Odour description (1% solution in EtOH on paper blotter, 24 h): powdery, vanilla, creamy, chocolate.

### Example 59: 4-(4-((4-methyl-7-phenylhept-3-en-1-yl)oxy)phenyl)butan-2-one

The compound was obtained from 4-(4-hydroxyphenyl)butan-2-one (2.50 g, 15.2 mmol, 1.0 equiv) and (7-chloro-4-methylhept-4-en-1-yl)benzene (3.39 g, 15.2 mmol, 1.0 equiv) according to the procedure of example 42 as a colorless liquid (0.89 g, 17% yield, E/Z 90:10). ¹H NMR (400 MHz, CDCl₃, mixture of E/Z isomers): δ 7.31-7.23 (m, 2H), 7.23-7.13 (m, 3H), 7.12-7.03 (m, 2H), 6.86-6.74 (m, 2H), 5.34-5.06 (m, 1H), 3.98-3.84 (m, 2H), 2.90-2.77 (m, 2H), 2.75-2.66 (m, 2H), 2.62-2.53 (m, 2H), 2.52-2.38 (m, 2H), 2.16-2.00 (m, 5H), 1.78-1.68 (m, 2H), 1.65 (s, 3H) ppm. ¹³C NMR (101 MHz, CDCl₃, mixture of E/Z isomers): δ 208.1, 157.3, 142.5, 137.6, 132.8, 129.1, 128.5, 128.4, 128.3, 128.3, 128.2, 128.2, 125.9, 125.7, 125.6, 120.4, 119.7, 114.5, 114.5, 114.5, 67.7, 67.6, 45.4, 39.2, 35.8, 35.4, 32.1, 31.6, 30.8, 30.1, 29.7, 29.5, 28.9, 28.2, 28.0, 23.4, 16.1 ppm.

Odour description (1% solution in EtOH on paper blotter, 24 h): powdery fruity, raspberry.

### Example 60: 4-((4,8-dimethylnona-3,7-dien-1-yl)oxy)-3-ethoxybenzaldehyde

The compound was obtained from 3-ethoxy-4-hydroxybenzaldehyde (1.086 g, 6.54 mmol, 1.1 equiv) and (E)-4,8-dimethylnona-3,7-dien-1-ol (1.0 g, 5.94 mmol, 1.0 equiv) according to the procedure of example 1 as colorless liquid (1.0 mmol, 0.3 g, 16% yield).

¹H NMR (400 MHz, CDCl₃) δ 9.83 (s, 1H), 7.51-7.32 (m, 2H), 6.98-6.95 (m, 1H), 5.22 (t, J=7.1 Hz, 1H), 5.09 (t, J=6.6 Hz, 1H), 4.14 (q, J=7.0 Hz, 2H), 4.05 (q, J=6.9 Hz, 2H), 2.61-2.56 (m, 2H), 2.15-1.96 (m, 4H), 1.77-1.45 (m, 9H), 1.47 (t, J=7.0 Hz, 3H) ppm. ¹³C NMR (101 MHz, CDCl₃, E/Z mixtures) δ 191.0, 154.4, 154.4, 149.2, 138.7, 138.7, 131.8, 131.5, 129.9, 126.6, 124.1, 124.1, 119.5, 118.7, 111.8, 110.9, 68.8, 68.6, 64.6, 39.8, 32.1, 27.9, 27.8, 26.6, 26.6, 25.7, 25.7, 23.5, 17.7, 16.2, 14.7 ppm. GC/MS (EI): m/z (%): 316 (1) [M⁺], 166 (7), 151 (18), 109 (11), 95 (36), 69 (100).

Odour description (1% solution in EtOH on paper blotter, 24 h): gourmand (vanilla, rhum, vanilla bean) sweet (vanilla)

### Example 61: 4-allyl-1-((4,8-dimethylnona-3,7-dien-1-yl)oxy)-2-methoxybenzene

The compound was obtained from 4-allyl-2-methoxyphenol (0.5 g, 3.1 mmol, 1.0 equiv) and (E)-4,8-dimethylnona-3,7-dien-1-ol (0.5 g, 3.1 mmol, 1.0 equiv) according to the procedure of example 1 as colorless liquid (1.8 mmol, 0.6 g, 59% yield).

¹H NMR (400 MHz, CDCl₃) δ 6.74-6.60 (m, 3H), 5.91-5.82 (m, 1H), 5.14-4.91 (m, 4H), 3.89-3.74 (m, 5H), 3.24 (d, J=6.7 Hz, 2H), 2.49-2.42 (m, 2H), 2.03-1.91 (m, 4H), 1.68-1.45 (m, 9H). ¹³C NMR (101 MHz, CDCl₃, E/Z mixtures) δ 149.4, 149.4, 146.8, 146.8, 138.2, 138.1, 137.7, 132.8, 132.7, 131.7, 131.4, 124.2, 124.2, 120.5, 119.9, 119.1, 115.6, 113.2, 113.1, 112.4, 112.3, 68.8, 68.6, 56.0, 55.9, 39.8, 39.8, 32.1, 28.2, 28.1, 26.7, 26.6, 25.8, 25.7, 23.5, 17.7, 17.7, 16.2 ppm. GC/MS (EI): m/z (%): 314 (6) [M⁺], 177 (2), 164 (100), 149 (7), 115 (5), 95 (14), 69 (66).

Odour description (1% solution in EtOH on paper blotter, 24 h): spicy

### Example 62: methyl (E)-4-((4,8-dimethylnona-3,7-dien-1-yl)oxy)-2-hydroxy-3,6-dimethylbenzoate

The compound was obtained from methyl 2,4-dihydroxy-3,6-dimethylbenzoate (1.0 g, 5.1 mmol, 1.0 equiv) and (E)-4,8-dimethylnona-3,7-dien-1-ol (0.9 g, 5.1 mmol, 1.0 equiv) according to the process of example 1 as colorless liquid (1.2 mmol, 0.4 g, 23% yield).

¹H NMR (400 MHz, CDCl₃) δ 11.74 (s, 1H), 6.14 (s, 1H), 5.13 (t, J=7.1 Hz, 1H), 5.00 (t, J=6.4 Hz, 1H), 3.85 (q, J=6.6 Hz, 2H), 3.80 (s, 3H), 2.46-2.35 (m, 5H), 2.05-1.88 (m, 7H), 1.64-1.51 (m, 9H) ppm. ¹³C NMR (101 MHz, CDCl₃, E/Z mixtures) δ 172.6, 162.2, 161.0, 160.9, 141.0, 138.2, 138.0, 131.8, 131.4, 124.2, 124.1, 120.2, 119.4, 111.0, 106.8, 105.2, 68.0, 67.8, 51.7, 39.8, 32.1, 28.2, 28.1, 26.6, 26.6, 25.7, 25.7, 24.6, 23.4, 17.7, 17.6, 16.2, 7.9, 7.9 ppm. GC/MS (EI): m/z (%): 346 (3) [M⁺], 277 (2), 245 (5), 197 (38), 177 (16), 164 (24), 135 (14), 95 (20), 69 (100).

Odour description (1% solution in EtOH on paper blotter, 24 h): powdery (mossy)

### Example 63: (E)-3-ethoxy-4-((3-methyl-5-(2,2,3-trimethylcyclopent-3-en-1-yl)pent-4-en-2-yl)oxy)benzaldehyde

The compound was obtained from 3-ethoxy-4-hydroxybenzaldehyde (1.8 g, 11.0 mmol, 1.0 equiv) and (E)-4-(4-bromo-3-methylpent-1-en-1-yl)-1,5,5-trimethylcyclopent-1-ene (3.0 g, 11.0 mmol, 1.0 equiv) according to the procedure of example 1 as colorless liquid (1.1 mmol, 0.4 g, 10% yield).

¹H NMR (400 MHz, CDCl₃) δ 9.83 (s, 1H), 7.41 (d, J=7.5 Hz, 2H), 6.99-6.95 (m, 1H), 5.67-5.30 (m, 2H), 5.23 (s, 1H), 4.74-4.08 (m, 3H), 2.92-0.74 (m, 22H) ppm. GC/MS (EI): m/z (%): 356 (5) [M⁺], 281 (5), 207 (13), 191 (42), 166 (50), 149 (36), 137 (99), 109 (100), 90 (70), 55 (72).

Odour description (1% solution in EtOH on paper blotter, 24 h): woody, sandalwoody, hay

### Example 64: (Z)-3-ethoxy-4-(non-3-en-1-yloxy)benzaldehyde

The compound was obtained from 3-ethoxy-4-hydroxybenzaldehyde (5.0 g, 30.1 mmol, 1.0 equiv) and (Z)-1-bromonon-3-ene (6.2 g, 30.1 mmol, 1.0 equiv) according to the process of example 1 as colorless liquid (16.5 mmol, 4.8 g, 55% yield).

¹H NMR (400 MHz, CDCl₃) δ 9.73 (s, 1H), 7.33-7.30 (m, 2H), 6.86 (d, J=8.1 Hz, 1H), 5.50-5.32 (m, 1H), 4.11-3.90 (m, 4H), 2.56-2.50 (m, 2H), 2.02-1.97 (m, 2H), 1.37 (t, J=7.0 Hz, 3H), 1.33-1.14 (m, 6H), 0.79 (t, J=6.8 Hz, 3H) ppm. ¹³C NMR (101 MHz, CDCl₃) δ 190.9, 154.3, 149.1, 133.3, 130.0, 126.5, 123.9, 111.8, 110.8, 68.5, 64.5, 31.5, 29.3, 27.3, 27.2, 22.5, 14.7, 14.0 ppm. GC/M S (EI): m/z (%): 290 (11) [M+], 192 (1), 166 (100), 151 (7), 138 (57), 83 (42), 69 (73), 55 (60).

Odour description (1% solution in EtOH on paper blotter, 24 h): vanilla (powdery, spicy, creamy)

### Example 65: 2-ethoxy-4-methyl-1-((4,8,12-trimethyltrideca-3,7,11-trien-1-yl)oxy)benzene

The compound was obtained from 2-ethoxy-4-methylphenol (5.2 g, 27.4 mmol, 1.0 equiv) and 4,8,12-trimethyltrideca-3,7,11-trien-1-ol (7.8 g, 32.8 mmol, 1.2 equiv) according to the procedure of example 1 as colorless liquid (14.3 mmol, 5.3 g, 51% yield).

¹H NMR (400 MHz, CDCl₃) δ 6.78 (d, J=8.0 Hz, 1H), 6.74-6.59 (m, 2H), 5.23-5.07 (m, 3H), 4.06 (q, J=7.0 Hz, 2H), 3.94 (t, J=7.2 Hz, 2H), 2.55-2.49 (m, 2H), 2.27 (s, 3H), 2.17-1.90 (m, 8H), 1.73-1.60 (m, 12H), 1.43 (t, J=6.9 Hz, 3H). ¹³C NMR (101 MHz, CDCl₃, mixture of E/Z isomers) δ 148.7, 146.7, 138.0, 137.9, 135.1, 131.3, 130.7, 124.4, 124.1, 124.0, 121.1, 120.3, 119.5, 115.0, 115.0, 114.2, 69.0, 64.6, 39.8, 39.8, 32.1, 28.3, 28.2, 26.8, 26.7, 26.6, 25.7, 23.5, 21.0, 17.7, 16.2, 16.0, 15.0. GC/MS (EI): m/z (%): 370 (6) [M⁺], 234 (1), 214 (1), 152 (100), 137 (9), 124 (16), 81 (33), 69 (53).

Odour description (1% solution in EtOH on paper blotter, 24 h): phenolic (medicinal, ultravanil, balsamic vanilla bean, cough syrup)

### Example 66: methyl 2-hydroxy-3,6-dimethyl-4-((4,8,12-trimethyltrideca-3,7,11-trien-1-yl)oxy)benzoate

The compound was obtained from methyl 2,4-dihydroxy-3,6-dimethylbenzoate (6.6 g, 33.8 mmol, 1.0 equiv) and 4,8,12-trimethyltrideca-3,7,11-trien-1-ol (8.0 g, 33.8 mmol, 1.0 equiv) according to the procedure of example 1 as colorless liquid (28.0 mmol, 11.6 g, 83% yield). ¹H NMR (400 MHz, CDCl₃) δ 11.74 (s, 1H), 6.11 (s, 1H), 5.23-4.89 (m, 3H), 3.91-3.69 (m, 5H), 2.49-2.24 (m, 5H), 2.08-1.79 (m, 11H), 1.64-1.45 (m, 12H) ppm. ¹³C NMR (101 MHz, CDCl₃, mixture of E/Z isomers) δ 172.6, 162.3, 161.0, 140.0, 138.2, 138.0, 135.4, 135.1, 131.2, 131.2, 124.4, 124.4, 124.0, 123.9, 120.2, 119.5, 111.0, 106.7, 105.2, 68.0, 67.8, 51.6, 39.8, 32.1, 28.3, 28.1, 26.8, 26.7, 26.5, 25.7, 24.6, 23.5, 17.7, 16.2, 16.0, 7.9 ppm. GC/MS (EI): m/z (%): 414 (1) [M⁺], 389 (1), 244 (75), 217 (13), 201 (100), 173 (28), 145 (52), 119 (39), 55 (54).

Odour description (1% solution in EtOH on paper blotter, 24 h): powdery mossy (evernyl)

### Example 67: 4-(4-((4,8,12-trimethyltrideca-3,7,11-trien-1-yl)oxy)phenyl)butan-2-one

The compound was obtained from 4-(4-hydroxyphenyl)butan-2-one (4.5 g, 27.5 mmol, 1.0 equiv) and 4,8,12-trimethyltrideca-3,7,11-trien-1-ol (6.5 g, 27.5 mmol, 1.0 equiv) according to the procedure of example 1 as colorless liquid (20.0 mmol, 7.8 g, 74% yield).

¹H NMR (400 MHz, CDCl₃) δ 7.07 (d, J=8.6 Hz, 2H), 6.88-6.72 (m, 2H), 5.28-5.15 (m, 1H), 5.15-5.03 (m, 2H), 3.91-3.86 (m, 2H), 2.84-2.68 (m, 2H), 2.50-2.44 (m, 2H), 2.16-1.91 (m, 11H), 1.82-1.55 (m, 12H). ¹³C NMR (101 MHz, CDCl₃, mixture of E/Z isomers) δ 208.1, 157.4, 138.1, 137.9, 135.3, 135.1, 132.9, 131.3, 131.2, 129.2, 124.4, 124.4, 124.1, 124.0, 120.3, 119.5, 114.6, 114.6, 67.8, 67.7, 45.5, 39.7, 39.7, 32.1, 30.1, 29.0, 28.2, 28.1, 26.8, 26.7, 26.5, 26.5, 25.7, 23.5, 17.7, 16.2, 16.1, 16.0. GC/MS (EI): m/z (%): 414 (1) [M⁺], 382 (2), 246 (15), 219 (2), 187 (18), 164 (15), 82 (59), 69 (100).

Odour description (1% solution in EtOH on paper blotter, 24 h): fruity powdery (raspberry)

### Example 68: 3-ethoxy-4-((1-phenylbut-3-en-1-yl)oxy)benzaldehyde

The compound was obtained from 3-ethoxy-4-hydroxybenzaldehyde (5.0 g, 30.1 mmol, 1.0 equiv) and 1-phenylbut-3-en-1-ol (5.4 g, 36.1 mmol, 1.2 equiv) according to the procedure of example 1 as colorless liquid (16.9 mmol, 5.0 g, 56% yield).

¹H NMR (400 MHz, CDCl₃) δ 9.74 (s, 1H), 7.37-7.21 (m, 7H), 6.78 (d, J=8.2 Hz, 1H), 5.95-5.75 (m, 1H), 5.26-5.07 (m, 3H), 4.15 (q, J=7.0 Hz, 2H), 2.88-2.64 (m, 2H), 1.48 (t, J=7.0 Hz, 3H) ppm. ¹³C NMR (101 MHz, CDCl₃) δ 191.0, 153.6, 149.9, 140.5, 133.7, 130.3, 128.6, 127.9, 126.2, 126.1, 118.0, 115.1, 111.6, 81.4, 64.8, 42.7, 14.8 ppm. GC/MS (EI): m/z (%): 296 (1) [M⁺], 255 (1), 225 (8), 166 (6), 149 (6), 131 (100), 115 (20), 103 (8), 91 (63), 77 (12).

Odour description (1% solution in EtOH on paper blotter, 24 h): powdery (vanilla), green

### Example 69: methyl (Z)-4-(deca-1,7-dien-4-yloxy)-2-hydroxy-3,6-dimethylbenzoate

The compound was obtained from methyl 2,4-dihydroxy-3,6-dimethylbenzoate (6.0 g, 30.6 mmol, 1.0 equiv) and (Z)-deca-1,7-dien-4-ol (5.2 g, 33.6 mmol, 1.1 equiv) according to the procedure of example 1 as colorless liquid (12.3 mmol, 4.1 g, 41% yield).

¹H NMR (400 MHz, CDCl₃) δ 11.78 (s, 1H), 6.16 (s, 1H), 5.76-5.68 (m, 1H), 5.40-5.12 (m, 2H), 5.09-4.89 (m, 2H), 4.35-4.25 (m, 1H), 3.81 (s, 3H), 2.40-2.34 (m, 5H), 2.07-1.60 (m, 9H), 0.82 (t, J=7.5 Hz, 3H) ppm. ¹³C NMR (101 MHz, CDCl₃) δ 171.5, 161.5, 159.2, 138.7, 132.7, 131.6, 126.9, 116.7, 110.9, 106.9, 104.0, 75.3, 50.7, 37.3, 32.7, 23.6, 21.9, 19.4, 13.2, 7.0 ppm. GC/MS (EI): m/z (%): 332 (10) [M⁺], 301 (1), 259 (1), 196 (29), 164 (100), 136 (22), 95 (12).

Odour description (1% solution in EtOH on paper blotter, 24 h): powdery green marine (evernyl)

### Example 70: Application in Liquid Detergent

### a) Sample Preparation

0.2% by weight of ethyl vanillin (3-ethoxy-4-hydroxybenzaldehyde, sample A) or (Z)-3-ethoxy-4-(pentadec-3-en-6-yloxy)benzaldehyde (compound of example 28, sample B) were incorporated respectively into unperfumed liquid detergent base by magnetic stirring at room temperature for 24 h.

### b) Colour Stability

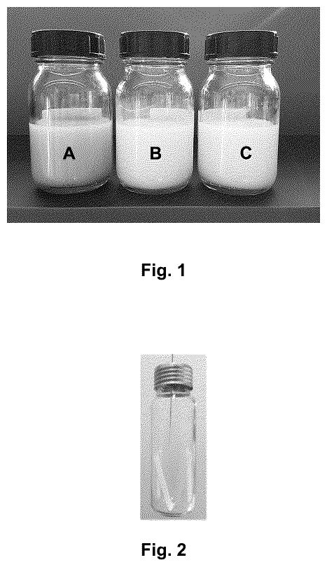

The above prepared samples were visually inspected in comparison to an unperfumed base sample (sample C). The ethylvanillin sample (A) showed strong purple coloration, whereas the sample containing the precursor (B) was optically identical to the unperfumed base (C). All three samples are shown in FIG. 1.

### c) Washtest and Sensory Evaluation

A 40° C. machine wash cycle was performed using 55 g of the above prepared liquid detergent samples A and B and odour-neutral cotton/elastan mixed fabric T-shirts. The wet and line-dried fabric (1 and 4 days) was assessed by a panel of 4-6 experts with regard to odour intensity and quality. The odour intensity was recorded on an intensity scale of 0 (odourless) to 5 (extremely strong). As can be seen from Table 1 below, virtually no odour was released from the fabric washed with the colored ethyl vanillin detergent base, whereas the fabric washed with the color-neutral detergent base containing (Z)-3-ethoxy-4-(pentadec-3-en-6-yloxy)benzaldehyde exhibited a mandarin, vanilla and powdery fragrance after 1 and 3 days.

TABLE 1

| Sample | wet | 1 day | 4 days |
|---|---|---|---|
| Ethylvanillin (A) | 1.2 | 0.9 | 1 |
| (Z)-3-ethoxy-4-(pentadec-3- | 2.1 | 2.3 | 2.6 |
| en-6-yloxy)benzaldehyde (B) | | | |

The experiment demonstrates that free ethylvanillin is not suitable for application in liquid detergent, as it colors the base and, due to its high water solubility, does not impart any fragrance to washed and dried fabric. These technical limitations are overcome by the compound of the present invention, which is not colorizing and imparts a mandarin and vanilla-powdery fragrance on dry fabric.

### Example 71: Comparison of Regioisomeric Side Chain Olefins

In order to highlight the importance of the position of the double bond, a comparative assessment in liquid detergent application (as described in Example 70a and c) was carried out with ethylvanillin ethers (compounds of formula IV), having unsaturated side chains with the double bond placed in positions Δ-2 or Δ-4 in comparison to the compounds of the current invention which have the double bond positioned at Δ-3.

The compound with the double bond in Δ-2 position, 3-ethoxy-4-(((2Z,5Z)-octa-2,5-dien-1-yl)oxy)benzaldehyde, was prepared by stirring ethyl vanillin with (Z)-3-chloroocta-1,5-diene in the presence of potassium carbonate (1.5 equiv) and tetrabutyl ammonium iodide (0.15 equiv.) in acetonitrile under reflux for 40 h. Standard workup and purification yielded 3-ethoxy-4-(((2Z,5Z)-octa-2,5-dien-1-yl)oxy)benzaldehyde as a pale yellow oil (13%). GC/MS (EI, 70 eV): 274 (1, [M]⁺), 166 (19), 137 (25), 109 (32), 79 (48), 67 (100), 55 (37).

The compound with the double bond in A-4 position, (Z)-3-ethoxy-4-(hexadec-3-en-7-yloxy)benzaldehyde, was prepared by reacting (Z)-hexadec-3-en-7-ol (from the reaction of (Z)-hex-3-en-1-ylmagnesium chloride with decanal) and ethyl vanillin according to the procedure of example 1 to yield (Z)-3-ethoxy-4-(hexadec-3-en-7-yloxy)benzaldehyde as a liquid (32%). GC/MS (EI, 70 eV): 388 (<1, [M]⁺), 222 (1), 166 (100), 138 (18), 109 (12), 97 (31), 83 (43), 69 (42), 55 (43).

The odour intensity was recorded on an intensity scale of 0 (odorless) to 5 (extremely strong). The results are summarized in Table 2.

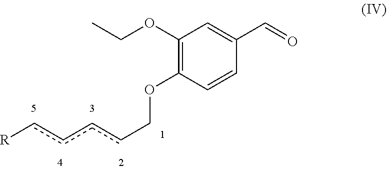

TABLE 2

| | | position | | | | |
|---|---|---|---|---|---|---|
| | | of | | | | |
| | | double | | 1 | 3 | |
| Entry | Compound | bond | wet | day | days | odour on dry fabric |
| 1 | 3-ethoxy-4-(((2Z,5Z)-octa-2,5- | Δ-2 | 0.9 | 2.8 | 2.3 | metallic |
| | dien-1-yl)oxy)benzaldehyde | | | | | |
| 2 | 3-ethoxy-4-(((3Z,6Z)-nona-3,6- | Δ-3 | 2.7 | 3.4 | 4.1 | vanilla, green |
| | dien-1-yl)oxy)benzaldehyde | | | | | |
| | (Example 30) | | | | | |
| 3 | (Z)-3-ethoxy-4-(hexadec-3-en- | Δ-4 | 1.1 | 1.2 | 1.6 | rubbery, metallic |
| | 7-yloxy)benzaldehyde | | | | | |

The results show that only the compound of the present invention with the double bond in the Δ-3 position of the side chain (homoallylic ether, entry 2) releases a strong and pleasant odour on dry fabric, whereas comparable compounds with the double bond in the Δ-2 or Δ-4 position (entries 1 and 3), respectively, release only weak and unpleasant odours.

### Example 72: Application in Liquid Detergent: Generation of Different Odour Profiles

Heavy duty liquid detergent samples were prepared and used in washtests and subsequent sensory evaluation of the washed and dried fabric as described in Example 60a and c. The odour intensity was recorded on an intensity scale of 0 (odourless) to 5 (extremely strong). The results are summarized in Table 3.

TABLE 3

| | | | 1 | 4 | odour on |
|---|---|---|---|---|---|
| Entry | | wet | day | days | dry fabric |
| 1 | (Z)-3-ethoxy-4-(pentadec-3-en-6- | 2.1 | 2.3 | 2.6 | mandarin, vanilla, |
| | yloxy)benzaldehyde | | | | powdery |
| 2 | 3-ethoxy-4-(((3Z,6Z)-nona-3,6-dien-1- | 2.7 | 3.4 | 4.1 | green, powdery, |
| | yl)oxy)benzaldehyde | | | | vanilla |
| 3 | (E)-3-ethoxy-4-((4-methylnon-3-en-1- | 2.1 | 2.8 | 3.3 | vanilla, aldehydic, |
| | yl)oxy)benzaldehyde | | | | green |
| 4 | (Z)-4-(deca-1,7-dien-4-yloxy)-3- | 0.4 | 3.0 | 3.7 | green, violet, |
| | ethoxybenzaldehyde | | | | cucumber, vanilla |
| 5 | 3-methoxy-4-(tetradec-1-en-4- | 2.2 | 3.0 | 3.2 | vanilla, mandarin, |
| | yloxy)benzaldehyde | | | | citrus, madeleine |
| 6 | (E)-4-(4-((4-methyl-6-phenylhex-3-en-1- | 1.6 | 2.9 | 3.3 | raspberry, sweet |
| | yl)oxy)phenyl)butan-2-one | | | | |
| 7 | (E)-3-ethoxy-4-((4-methyl-6-phenylhex-3- | 1.5 | 2.7 | 3.0 | sweet, vanilla, |
| | en-1-yl)oxy)benzaldehyde | | | | raspberry |
| 8 | 4-allyl-2-methoxy-1-(tridec-1-en-4- | 2.1 | 2.1 | 2.7 | spicy, vanilla, |
| | yloxy)benzene | | | | green, oranger |
| 9 | methyl (Z)-4-(deca-1,7-dien-4-yloxy)-2- | 0.5 | 1.9 | 2.5 | mossy, woody |
| | hydroxy-3,6-dimethylbenzoate | | | | |

The results illustrate that strong fragrance intensities are achieved on dry fabric with compounds of the current invention. The odour direction of a given parent phenol (e.g. ethylvanillin) can be modulated or even dominated by odorants released from the side chain. It also illustrates that other aromas can be generated through the appropriate combination of phenolic and side chain moieties. For example, the popular aroma of madeleine can be released by the compound of example 38, as shown in entry 5 in Table 3).

### Example 73: Application in Shampoo Base

Hair shampoo samples (20 g) were prepared by adding 0.2% by weight of ethyl vanillin or compounds of the present invention to unperfumed clear hair shampoo base and mixing on a bottle roller for 24 h at room temperature. Odour-neutral human hair swatches were wetted with warm tap water and lathered delicately with 2 mL of the above hair shampoo samples for 30 sec by hand wearing gloves. The lathered hair swatches were left in a plastic bowl for 2 min, then rinsed under running tap water for 20 sec. After removing excess water by squeezing the hair swatches between two fingers, they were left to dry in open air. The wet and dried hair swatches (1 and 3 days) were assessed by a panel of 4-6 experts with regard to odour intensity and quality. The odour intensity was recorded on an intensity scale of 0 (odourless) to 5 (extremely strong). The results are summarized in Table 4.

TABLE 4

| Entry | | wet | 1 day | 3 days | odour on dry hair |
|---|---|---|---|---|---|
| 1 | ethylvanillin | 4.2 | 3.0 | 2.5 | vanilla, powdery |
| 2 | (Z)-3-ethoxy-4-(pentadec-3-en-6- | 1.7 | 3.5 | 4.4 | mandarin, vanilla |
| | yloxy)benzaldehyde (Example 28) | | | | |
| 3 | 4-(4-(tridec-1-en-4- | 2.3 | 1.9 | 1.6 | raspberry, fruity, |
| | yloxy)phenyl)butan-2-one (Example | | | | floral |
| | 36) | | | | |
| 4 | 3-ethoxy-4-((4-methyl-6-phenylhex-3- | 1.4 | 2.7 | 2.1 | vanilla, raspberry, |
| | en-1-yl)oxy)benzaldehyde (Example | | | | sweet, floral |
| | 47) | | | | |
| 5 | 3-ethoxy-4-((4-methyldodec-3-en-1- | 1.1 | 2.7 | 2.9 | vanilla, sweet, |
| | yl)oxy)benzaldehyde (Example 90) | | | | chocolate |
| 6 | 3-ethoxy-4-((4-methylnon-3-en-1- | 1.0 | 1.4 | 1.9 | vanilla, sweet |
| | yl)oxy)benzaldehyde (Example 42) | | | | |
| 7 | 4-(4-((6-(4-methoxyphenyl)-4- | 1.1 | 2.8 | 3.0 | fruity, raspberry, |
| | methylhex-3-en-1- | | | | sweet |
| | yl)oxy)phenyl)butan-2-one (Example | | | | |
| | 55) | | | | |
| 8 | 4-((5,9-dimethyldeca-3,8-dien-1- | 0.7 | 2.7 | 3.6 | vanilla, powdery, |
| | yl)oxy)-3-ethoxybenzaldehyde | | | | spicy |
| | (Example 84) | | | | |

The experiment demonstrates that the precursors of the current invention are capable of releasing fragrance on dry hair, which is becoming stronger over time, whereas the odour of free ethylvanillin fades during the same time period.

### Example 74: Headspace Analysis of Released Volatiles

A solution of the compound of formula (I) (100 μg, 1.0 mg/mL solution in MTBE) was evenly applied through a displacement pipette to a paper strip (1 cm×8 cm). The paper strip was left to dry (1 h at room temperature), then folded in the middle and inserted in “V”-shape into a 20 mL headspace vial (FIG. 2). The vial was closed with a septum screwcap and placed in a cabinet equipped with a fluorescent daylight imitation lamp and mirrored walls for 24 h (temperature 27-28° C.).

Extraction and analysis of the released organic volatiles was effected in automated mode on a Trace 1310 gas chromatograph equipped with a TriPlus RSH autosampler (Thermo Fisher Scientific), coupled to a ISQ LT mass spectrometer (Thermo Fisher Scientific). The extraction of the organic volatiles from the headspace with SPME (solid phase microextraction) was carried out during 1 h at 40° C. using a 50/30 μm divinylbenzene/carboxen/polydimethylsiloxane SPME fibre (DVB/CAR/PDMS, Supelco P/N 57298-U), and subsequently desorbed in splitless mode at 250° C. for 1 min. GC-MS analysis (gas chromatography with mass spectrometric detection) was carried out with a VF-WAXms capillary column (Agilent, 30 m length, 0.25 mm I.D., 0.25 μm film thickness). Helium was used as a carrier gas with a constant flow rate of 1 ml/min. The GC oven temperature was programmed from 35° C. with 2-min hold to 250° C. at 5° C./min with a 10-min final temperature hold. The mass spectrometer was operated at 70 eV in EI mode over a m/z range of 33-350, scanned at 0.2 s intervals and with a temperature of transfer line and ion source of 230° C. and 220° C., respectively. A list of identified volatiles released from the compounds of formula (I) is reported in the Table 5 (indicated are relative peak area %; in bold: main odor vector).

TABLE 5

| Precursor | Released Volatiles (MS-TIC peak area %) |
|---|---|
| (Z)-4-(deca-1,7-dien-4-yloxy)-3- | 2,6-Nonadienal (4.7%), ethylvanillin |
| ethoxybenzaldehyde | (0.03%) |
| (E)-3-ethoxy-4-(pentadec-3-en-6- | Decanal (4.7%), 2-dodecenal (1.2%), |
| yloxy)benzaldehyde | ethylvanillin (0.02%) |
| 4-(dec-1-en-4-yloxy)-3- | 2-Nonenal (4.1%), 1-decen-4-ol (25.5%), 3- |
| ethoxybenzaldehyde | decen-2-one (5.1%), ethylvanillin (0.05%) |
| 3-ethoxy-4-(((3Z,6Z)-nona-3,6-dien-1- | 2-Pentenal (13.1%), 1-penten-3-ol (11.8%), |
| yl)oxy)benzaldehyde | 2-hexenal (11.4%), nona-3,6-dien-1-ol, |
| | (8.0%), ethylvanillin (0.8%) |
| 3-ethoxy-4-((1-phenylbut-3-en-1- | benzaldehyde (12.7%), acetophenone |
| yl)oxy)benzaldehyde | (1.8%), 1-phenylbut-3-en-1-ol (21%), |
| | cinnamaldehyde (0.3%), ethylvanillin |
| | (0.3%) |
| methyl 2-((4-methyl-6-phenylhex-3-en-1- | methyl salicylate (21.7%), 4-phenyl-2- |
| yl)oxy)benzoate (example 56) | butanone (5.2%) |
| 3-ethoxy-4-((4-(naphthalen-2-yl)pent-3-en- | 2-acetyl naphtalene (“Oranger Crystals”, |
| 1-yl)oxy)benzaldehyde (example 51) | 12.8%), ethylvanillin (1.0%) |
| 3-ethoxy-4-((4-methyl-6-phenylhex-3-en-1- | benzylacetone (45.5%), |
| yl)oxy)benzaldehyde (example 47) | ethylvanillin (0.5%) |

It should be noted that the indicated relative peak area % values are not related to molar release rates. The relative amount of each volatile in the gas phase depends mainly on volatility. Therefore, the sensory active phenols appear as minor components.

The results show that besides phenolic fragrant compounds like ethylvanillin or methyl salicylate, further sensorially active aldehydes, ketones and alcohols are released from compounds of formula (I).

The sensorially active phenols were observed also in samples which were not exposed to a light source under otherwise identical conditions indicating that the release can occur upon exposure to ambient air by oxidation in the presence or absence of light.

### Example 75: Assessment of Biodegradability

The results of the biodegradability assessment by the manometric respirometry test (OECD guideline for the testing of materials No. 301F, Paris 1992) are summarized in Table 6.

TABLE 6

| Compound | | Biodegra- | |
|---|---|---|---|
| of example | Compound name | dation in % | result |
| 29 | (E)-3-ethoxy-4-(pentadec-3-en-6- | 70 | inherently |
| | yloxy)benzaldehyde | | biodegradable |
| 17 | 3-ethoxy-4-(undec-3-en-6- | 86 | inherently |
| | yloxy)benzaldehyde | | biodegradable |
| 1 | methyl (Z)-12-(2-ethoxy-4- | 90 | readily |
| | formylphenoxy)octadec-9-enoate | | biodegradable |
| 2 | methyl (Z)-12-(4-(3- | 88 | readily |
| | oxobutyl)phenoxy)octadec-9-enoate | | biodegradable |

The results show that compounds of the present invention with varying phenol moieties and side chains are biodegradable. A compound can be classified biodegradable, if it reaches the pass level of 60% oxygen consumption of theory required for complete mineralization.

It is readily biodegradable, if the pass level is reached within 10 days within the 28-day period of the test. The 10-day window begins when the degree of biodegradation has reached 10%. If the pass level is obtained after 28-day period of the test, the compound can be classified as inherently biodegradable.

### Example 76: 4-(((3E,9Z)-dodeca-3,9-dien-1-yl)oxy)-3-ethoxybenzaldehyde

### a) (Z)-1-cyclopropylnon-6-en-1-ol

To the suspension of magnesium (4.16 g, 171 mmol, 1.2 equiv.) in tetrahydrofurane (THF, 10 mL) was added a solution of bromocyclopropane (20.7 g, 171 mmol, 1.2 equiv) in THF (150 mL) in the following way: the first 10 mL were added at once, then the reaction was started by heating to reflux with a heat gun, then the remainder of the solution was added dropwise under stirring. The solution was stirred for 1 h after completed addition, then cooled to 5° C. The solution of 6-(Z)-nonenal (20.0 g, 143 mmol, 1.0 equiv.) in THF (60 mL) was added dropwise at 5-15° C. After completed addition, the solution was stirred for 40 min, then poured on 2 N aq. HCl-solution (250 mL). The mixture was extracted with methyl t-butyl ether (MTBE, 200 mL), the organic layer was washed with water and brine and dried over MgSO₄. After removal of the solvent in a rotary evaporator, (Z)-1-cyclopropylnon-6-en-1-ol (26.25 g, >99%) was obtained as a clear yellow liquid. The product was used without purification in the subsequent step.

### b) Preparation of (9Z)-1-bromododeca-3,9-diene

To the above prepared product (26.25 g, 143 mmol) was added dropwise aq. HBr solution (48% wt/wt, 32.6 mL, 288 mmol, 2.0 equiv.). After completed addition, the dark brown mixture was stirred at 50° C. for 24 h, then poured on cold 2N aq. NaOH solution (250 mL). Workup was effected as described above to yield a clear, brown liquid (30.7 g, 87%). The product was purified by filtration over silica, eluting with n-pentane. (9Z)-1-bromododeca-3,9-diene was obtained as a clear, yellow liquid (20.2 g, 57%, purity 89% according to GC-MS).

¹H-NMR (500 MHz, CDCl₃) 5.52-5.59 (m, 1H), 5.31-5.46 (m, 3H), 3.39 (t, J=7.2 Hz, 2H), 2.45-2.69 (m, 2H), 1.97-2.20 (m, 6H), 1.30-1.52 (m, 4H), 0.98 (t, J=7.6 Hz, 3H). ¹³C-NMR (126 MHz, CDCl₃) 133.9 (s), 131.7 (s), 129.1 (s), 126.5 (s), 36.1 (t), 32.9 (t), 32.4 (t), 29.2 (t), 28.9 (t), 26.9 (t), 20.5 (t), 14.4 (q). GC/MS (EI): m/z (%) 246 (<1, [M⁺]), 204 (3), 188 (2), 160 (7), 110 (55), 95 (33), 81 (90), 67 (100), 55 (56), 41 (96).

### c) Preparation of 4-(((3E,9Z)-dodeca-3,9-dien-1-yl)oxy)-3-ethoxybenzaldehyde

To the solution of ethyl vanillin (1.13 g, 6.8 mmol, 1 equiv) in dimethyl formamide (DMF, 50 mL) was added K₂CO₃ (1.88 g, 13.6 mmol, 2 equiv.) followed by (9Z)-1-bromododeca-3,9-diene (1.83 g, 4.5 mmol, 1.1 equiv, as prepared above). The mixture was heated under stirring to 80° C. for 24 h, then cooled to room temperature and poured on water (100 mL). Workup was effected as described in chapter 1.1. to yield a clear, yellow liquid (1.71 g). The crude product was purified by column chromatography on silica with heptane/MTBE 84:16 to afford 4-(((3E,9Z)-dodeca-3,9-dien-1-yl)oxy)-3-ethoxybenzaldehyde (0.82 g, 37%) as a clear, colourless liquid with a purity of 95% according to GC-MS.

¹H-NMR (500 MHz, CDCl₃) 9.85 (s, 1H), 7.41-7.45 (m, 2H), 6.98 (d, J=8.1 Hz, 1H), 5.56-5.65 (m, 1H), 5.46-5.54 (m, 1H), 5.29-5.44 (m, 2H), 4.16 (q, J=6.8 Hz, 2H), 4.12 (t, J=7.3 Hz, 2H), 2.58 (q, J=6.6 Hz, 2H), 1.95-2.15 (m, 6H), 1.48 (t, J=7.0 Hz, 3H), 1.30-1.45 (m, 4H), 0.97 (t, J=7.5 Hz, 3H). ¹³C-NMR (126 MHz, CDCl₃) 190.9 (d), 154.4 (s), 149.2 (s), 133.8 (d), 131.7 (d), 130.0 (s), 129.1 (d), 126.6 (d), 124.8 (d), 111.9 (d), 111.0 (d), 68.9 (t), 64.6 (t), 32.5 (t), 32.3 (t), 29.2 (t), 28.9 (t), 26.9 (t), 20.5 (t), 14.7 (q), 14.4 (q).

GC/MS (EI): m/z (%) 330 (11, [M⁺]), 166 (77), 151 (6), 149 (9), 138 (43), 123 (32), 109 (48), 97 (12), 95 (75), 81 (90), 69 (65), 67 (80), 55 (100), 41 (95).

Odour description (1% solution in EtOH on paper blotter, 24 h): green, melon, powdery, vanilla.

### Example 77: methyl 4-(((3E,9Z)-dodeca-3,9-dien-1-yl)oxy)-2-hydroxy-3,6-dimethylbenzoate

The procedure described in example 76c) was repeated with methyl 2,4-dihydroxy-3,6-dimethylbenzoate (2.0 g, 10 mmol), (9Z)-1-bromododeca-3,9-diene (2.70 g, 11.0 mmol, 1.1 equiv, as prepared in example 76b)) and K₂CO₃ (2.80 g, 20.0 mmol, 2 equiv.). After workup and purification (flash chromatography on silica, heptane/MTBE 6:1 to 1:1, followed by removal of volatile impurities at 110° C./0.04 mbar in a Kugelrohr oven), methyl 4-(((3E,9Z)-dodeca-3,9-dien-1-yl)oxy)-2-hydroxy-3,6-dimethylbenzoate (0.75 g, 19%) was obtained as clear, slightly yellow liquid with a purity of >99% according to GC-MS.

¹H-NMR (400 MHz, CDCl₃) 11.85 (s, 1H), 6.27 (s, 1H), 5.45-5.63 (m, 2H), 5.30-5.43 (m, 2H), 4.02 (t, J=6.7 Hz, 2H), 3.94 (s, 3H), 2.46-2.60 (m, 5H), 2.01-2.14 (m, 9H), 1.25-1.53 (m, 4H), 0.98 (t, J=7.5 Hz, 3H). ¹³C-NMR (101 MHz, CDCl₃) 172.6 (s), 162.2 (s), 160.9 (s), 140.0 (s), 133.4 (d), 131.7 (d), 129.1 (d), 125.4 (d), 111.1 (s), 106.8 (d), 105.3 (s), 67.9 (t), 51.7 (q), 32.6 (t), 32.6 (t), 29.2 (t), 29.0 (t), 26.9 (t), 24.6 (q), 20.5 (t), 14.4 (q), 7.9 (q). GC/MS (EI): m/z (%) 360 (11, [M⁺]), 196 (90), 164 (100), 136 (22), 81 (31), 69 (28), 67 (32), 55 (42), 41 (51).

Odour description (1% solution in EtOH on paper blotter, 24 h): powdery, oakmoss.

### Example 78: 4-(deca-1,7-dien-4-yloxy)-3-ethoxybenzaldehyde

### a) Deca-1,7-dien-4-ol

To a solution of allylmagnesium bromide (63 mL, 107 mmol, 0.5 M) in THF (150 mL) was added hept-4-enal (10.0 g, 89 mmol) slowly at 10° C. and stirred for 3 hours at rt. The mixture was quenched by adding NH₄Cl solution, and extracted with MTBE (3*100 mL), the organic layers were combined, dried over MgSO₄, filtered and solvent was removed by rotary evaporation, the residue was purified by column chromatography on silica gel (PE:MTBE=4:1) to give deca-1,7-dien-4-ol (7.6 g, yield: 55%) as colorless liquid.

¹H NMR (400 MHz, CDCl₃, mixture of E/Z isomers) δ 5.90-5.70 (m, 1H), 5.58-5.28 (m, 2H), 5.20-5.07 (m, 2H), 3.73-3.55 (m, 1H), 2.35-2.22 (m, 1H), 2.21-1.95 (m, 5H), 1.88 (d, J=4.2 Hz, 1H), 1.60-1.47 (m, 2H), 0.96 (t, J=7.5 Hz, 3H) ppm. ¹³C NMR (101 MHz, CDCl₃, mixture of E/Z isomers) δ 134.9, 134.8, 132.6, 132.3, 128.6, 128.5, 118.0, 117.9, 70.3, 41.9, 41.9, 36.7, 36.5, 28.8, 25.6, 23.4, 20.5, 14.3, 13.9 ppm. GC/MS (EI): m/z (%): 154 (1) [M⁺], 136 (3), 95 (100), 69 (82), 55 (24).

### b) 4-(Deca-1,7-dien-4-yloxy)-3-ethoxybenzaldehyde

4-(Deca-1,7-dien-4-yloxy)-3-ethoxybenzaldehyde was obtained from 3-ethoxy-4-hydroxybenzaldehyde (5.9 g, 36 mmol) and deca-1,7-dien-4-ol (5.0 g, 32 mmol) according to the process of example 1 as light yellow liquid (6.1 g, 62% yield).

¹H NMR (400 MHz, CDCl₃, mixture of E/Z isomers) δ 9.74 (s, 1H), 7.38-7.23 (m, 2H), 6.89 (d, J=8.7 Hz, 1H), 5.83-5.73 (m, 1H), 5.41-5.20 (m, 2H), 5.12-4.95 (m, 2H), 4.42-4.26 (m, 1H), 4.04 (q, J=7.0 Hz, 2H), 2.52-2.29 (m, 2H), 2.15-1.95 (m, 2H), 1.87-1.56 (m, 4H), 1.36 (t, J=7.0 Hz, 3H), 0.90-0.77 (m, 3H) ppm. ¹³C NMR (101 MHz, CDCl₃, mixture of E/Z isomers) δ 190.9, 154.1, 150.1, 133.8, 133.7, 133.0, 132.6, 130.2, 128.0, 127.9, 126.2, 117.8, 117.8, 114.7, 114.5, 111.8, 111.7, 78.6, 78.5, 64.6, 38.3, 38.2, 33.7, 33.6, 28.3, 25.6, 22.9, 20.4, 14.7, 14.2, 13.9 ppm. GC/MS (EI): m/z (%): 302 (7) [M⁺], 166 (100), 138 (51), 95 (31), 81 (25), 67 (23).

Odour description (1% solution in EtOH on paper blotter, 24 h): powdery, vanilla

### Example 79: 4-((6-(benzo[d][1,3]dioxol-5-yl)-4-methylhex-3-en-1-yl)oxy)-3-ethoxybenzaldehyde

### a) 5-(6-bromo-3-methylhex-3-en-1-yl)benzo[d][1,3]dioxole

To a suspension of magnesium (1.64 g, 67.6 mmol, 1.3 equiv) overlayed with THF (5 mL) was added dropwise a solution of bromocyclopropane (7.55 g, 62.4 mmol, 1.2 equiv) in THF (20 mL), after having started the Grignard reaction using a catalytic amount of iodine and by heating to reflux with a heatgun. The reaction mixture was refluxed for 40 minutes. Then, a solution of 4-(benzo[d][1,3]dioxol-5-yl)butan-2-one (10.0 g, 52.0 mmol, 1.0 equiv) in THF (75 mL) was added dropwise at 10-15° C. over 25 minutes to the Grignard reagent. After stirring for 1.5 hour at room temperature, a solution of 62% sulfuric acid (56 g, 6.8 equiv, solution in water) was added dropwise at room temperature and the reaction mixture was stirred for 30 minutes at room temperature. After quenching with water (50 mL), the reaction mixture was extracted with MTBE (3×30 mL). The combined organic layers were washed with water (3×50 mL), brine (50 mL), dried over MgSO₄ and the solvent was removed under reduced pressure. The crude product was purified by column chromatography (heptane/MTBE 8:2) to give 5-(6-bromo-3-methylhex-3-en-1-yl)benzo[d][1,3]dioxole (7.76 g, 50 yield, E/Z 80:17) as a yellow liquid.

¹³C NMR (101 MHz, CDCl₃, mixture of E/Z isomers): δ 147.5, 147.4, 145.6, 145.5, 137.8, 137.5, 135.9, 135.8, 136.9, 122.4, 121.5, 121.1, 121.0, 108.9, 108.9, 108.8, 109.0, 108.1, 108.0, 100.7, 41.7, 34.3, 34.2, 33.9, 32.8, 32.7, 31.6, 31.5, 23.4, 16.4 ppm.

### b) 4-((6-(benzo[d][1,3]dioxol-5-yl)-4-methylhex-3-en-1-yl)oxy)-3-ethoxybenzaldehyde

The compound was obtained from 3-ethoxy-4-hydroxybenzaldehyde (1.97 g, 11.9 mmol, 1.0 equiv) and 5-(6-bromo-3-methylhex-3-en-1-yl)benzo[d][1,3]dioxole (3.88 g, 13.1 mmol, 1.1 equiv) according to the procedure of example 42 as an orange liquid (3.14 g, 69% yield, E/Z 80:20).

¹H NMR (400 MHz, CDCl₃, mixture of E/Z isomers): δ 9.87-9.80 (m, 1H), 7.45-7.37 (m, 2H), 6.95 (d, J=8.3 Hz, 0.8H), 6.89 (d, J=8.1 Hz, 0.2H), 6.73-6.57 (m, 3H), 5.92-5.84 (m, 2H), 5.28-5.14 (m, 1H), 4.20-4.09 (m, 2H), 4.01 (t, J=7.2 Hz, 1.6H), 3.88 (t, J=7.2 Hz, 0.4H), 2.70-2.43 (m, 4H), 2.37-2.23 (m, 2H), 1.81-1.69 (m, 3H), 1.52-1.42 (m, 3H) ppm. ¹³C NMR (101 MHz, CDCl₃, mixture of E/Z isomers): δ 190.9, 154.3, 154.2, 149.1, 149.0, 147.4, 147.3, 145.5, 145.4, 137.7, 137.5, 135.9, 135.8, 129.9, 126.5, 121.1, 121.1, 121.0, 120.4, 119.5, 111.6, 110.7, 108.8, 108.8, 108.0, 107.9, 100.6, 100.6, 68.5, 68.4, 64.4, 41.8, 34.2, 34.2, 33.9, 33.8, 31.8, 28.9, 27.8, 27.7, 23.4, 22.6, 16.2, 14.6, 14.6, 14.0 ppm.

Odour description (1% solution in EtOH on paper blotter, 24 h): sweet powdery, creamy, vanilla, vanitrope.

### Example 80: 4-(4-((6-(benzo[d][1,3]dioxol-5-yl)-4-methylhex-3-en-1-yl)oxy)phenyl)butan-2-one

The compound was obtained from 4-(4-hydroxyphenyl)butan-2-one (1.86 g, 11.3 mmol, 1.0 equiv) and 5-(6-bromo-3-methylhex-3-en-1-yl)benzo[d][1,3]dioxole (3.70 g, 12.4 mmol, 1.1 equiv) according to the procedure of example 42_as a colorless liquid (2.00 g, 46% yield, E/Z 80:20).

¹H NMR (400 MHz, CDCl₃, mixture of E/Z isomers): δ 7.12-7.07 (m, 2H), 6.84-6.77 (m, 2H), 6.74-6.67 (m, 2H), 6.65-6.61 (m, 1H), 5.91 (s, 1.6H), 5.87 (s, 0.4H), 5.27-5.19 (m, 1H), 3.88 (t, J=7.1 Hz, 1.6H), 3.76 (t, J=7.0 Hz, 0.4H), 2.87-2.82 (m, 2H), 2.77-2.62 (m, 4H), 2.53-2.23 (m, 4H), 2.14 (s, 3H), 1.78-1.68 (m, 3H) ppm. ¹³C NMR (101 MHz, CDCl₃, mixture of E/Z isomers): δ 208.1, 157.3, 147.4, 147.4, 145.5, 145.4, 137.1, 136.0, 135.9, 132.8, 129.1, 129.1, 121.1, 121.0, 121.0, 120.1, 114.5, 114.4, 108.9, 108.9, 108.8, 108.0, 108.0, 108.0, 100.7, 100.6, 67.6, 67.5, 45.4, 41.8, 34.3, 30.1, 28.9, 28.1, 27.8, 28.0, 27.8, 23.4, 16.3 ppm.

Odour description (1% solution in EtOH on paper blotter, 24 h): fruity, raspberry, grenadine, berryflor, fresh, transparent.

### Example 81: 3-ethoxy-4-((4-methyl-6-(2,6,6-trimethylcyclohex-1-en-1-yl)hex-3-en-1-yl)oxy)benzaldehyde

### a) 2-(6-bromo-3-methylhex-3-en-1-yl)-1,3,3-trimethylcyclohex-1-ene

Magnesium turnings (1.50 g, 61.8 mmol, 1.2 equiv) were added to a 350 ml-sulfonation flask and overlayed with THF (10 mL). Then 10% of a solution of bromocyclopropane (7.47 g, 61.8 mmol, 1.2 equiv) in THF (40 mL) were added. Afterwards the Grignard was started with a heating gun and a few iodine crystals. Then the remaining THF solution of bromocyclopropane was added dropwise. After the addition was completed, the reaction mixture was stirred for 1 hour at reflux. Then, a solution of 4-(2,6,6-trimethylcyclohex-1-en-1-yl)butan-2-one (10.0 g, 51.5 mmol, 1.0 equiv) in THF (40 mL) was added dropwise at 0° C. (the temperature rose to 13° C.). After the addition was completed, the cooling bath was removed and the reaction mixture was stirred for 16 hours at room temperature. Then 63% aq. sulfuric acid (12.0 mL, 139 mmol, 2.7 equiv) was added dropwise at 3° C. to the reaction mixture (the temperature rose to 18° C.). After stirring for 3 hours at room temperature, the reaction mixture was added to 120 mL of water for quenching and extracted with MTBE (150 mL). The organic layer was washed once with 2M NaOH (100 mL), water (150 mL) and brine (150 mL). The organic layer was dried over MgSO₄, filtered and the solvent was removed under reduced pressure. The crude was filtered over silica gel and eluted with 100% heptane to give 2-(6-bromo-3-methylhex-3-en-1-yl)-1,3,3-trimethylcyclohex-1-ene (6.96 g, 45% yield, mixture of isomers 63:14, 77% purity) as a yellow liquid.

GC/MS (EI) major isomer: m/z (%): 298 (1, [M]⁺·), 137 (100), 95 (50), 81 (32), 41 (15).

GC/MS (EI) minor isomer: m/z (%): 298 (0, [M]⁺·), 137 (100), 95 (52), 81 (32), 41 (14).

### b) 3-ethoxy-4-((4-methyl-6-(2,6,6-trimethylcyclohex-1-en-1-yl)hex-3-en-1-yl)oxy)benzaldehyde

The compound was obtained from 3-ethoxy-4-hydroxybenzaldehyde (1.90 g, 12 mmol, 1.0 equiv) and 2-(6-bromo-3-methylhex-3-en-1-yl)-1,3,3-trimethylcyclohex-1-ene (3.5 g, 12 mmol, 1.1 equiv) according to the procedure of example 42 as a yellow liquid (1.96 g, 44% yield, 3 isomers 84:13:3).

¹H NMR (400 MHz, CDCl₃, mixture of isomers): δ 9.84 (s, 1H), 7.45-7.39 (m, 2H), 7.00-6.96 (m, 1H), 5.27-5.19 (m, 1H), 4.18-4.06 (m, 4H), 2.62-2.56 (m, 2H), 2.15-2.02 (m, 4H), 1.97-1.89 (m, 2H), 1.81-1.77-1.71 (m, 3H), 1.65-1.54 (m, 5H), 1.50-1.45 (m, 3H), 1.45-1.40 (m, 2H), 1.02-0.99 (m, 6H) ppm. ¹³C NMR (101 MHz, CDCl₃, mixture of isomers): δ 190.9, 154.4, 149.2, 149.1, 139.6, 139.3, 136.9, 130.0, 129.9, 127.3, 127.1, 126.6, 126.5, 119.1, 117.9, 111.9, 111.8, 111.0, 110.9, 68.9, 68.6, 64.6, 40.2, 39.8, 39.8, 35.0, 34.9, 32.7, 32.7, 31.9, 29.0, 28.6, 28.6, 27.9, 27.7, 27.3, 23.4, 22.7, 19.9, 19.8, 19.5, 16.3, 14.7, 14.6, 14.1 ppm.

Odour description (1% solution in EtOH on paper blotter, 24 h): powdery, vanilla.

### Example 82: 4-(4-((4-methyl-6-(2,6,6-trimethylcyclohex-1-en-1-yl)hex-3-en-1-yl)oxy)phenyl)butan-2-one

The compound was obtained from 4-(4-hydroxyphenyl)butan-2-one (1.90 g, 11.6 mmol, 1.0 equiv) and 2-(6-bromo-3-methylhex-3-en-1-yl)-1,3,3-trimethylcyclohex-1-ene (3.46 g, 11.6 mmol, 1.1 equiv) according to the procedure of example 42 as a brown liquid (1.30 g, 29% yield, 3 isomers 83:13:4).

¹H NMR (400 MHz, CDCl₃, mixture of isomers): δ 7.11-7.07 (m, 2H), 6.85-6.80 (m, 2H), 5.30-5.14 (m, 1H), 3.94-3.90 (m, 2H), 2.86-2.81 (m, 2H), 2.74-2.70 (m, 2H), 2.53-2.46 (m, 2H), 2.13 (s, 3H), 2.12-2.00 (m, 4H), 1.98-1.89 (m, 2H), 1.80-1.68 (m, 3H), 1.68-1.54 (m, 5H), 1.46-1.40 (m, 2H), 1.03-0.99 (m, 6H) ppm. ¹³C NMR (101 MHz, CDCl₃, mixture of isomers): δ 208.1, 157.4, 138.9, 138.7, 137.0, 132.8, 129.1, 127.2, 127.0, 119.8, 119.0, 118.7, 114.6, 67.9, 67.7, 45.4, 40.2, 39.8, 39.8, 35.0, 34.9, 32.8, 32.7, 30.1, 28.9, 28.6, 28.6, 28.2, 27.7, 27.3, 23.3, 19.9, 19.8, 19.5, 16.3, 16.2 ppm.

Odour description (1% solution in EtOH on paper blotter, 24 h): fruity, sweet, powdery, raspberry ketone.

### Example 83: 3-ethoxy-4-((4-methyl-6-(2,6,6-trimethylcyclohex-1-en-1-yl)hexa-3,5-dien-1-yl)oxy)benzaldehyde

### a) 2-(6-bromo-3-methylhexa-1,3-dien-1-yl)-1,3,3-trimethylcyclohex-1-ene

Magnesium turnings (1.52 g, 62.4 mmol, 1.2 equiv) were added to a 350 ml-sulfonation flask and overlayed with THF (10 mL). Then 10% of a solution of bromocyclopropane (7.55 g, 62.4 mmol, 1.2 equiv) in THF (40 mL) were added. Afterwards the Grignard was started with a heating gun and a few iodine crystals. Then the remaining THF solution of bromocyclopropane was added dropwise. After the addition was completed, the reaction mixture was stirred for 1 hour at reflux. Then, a solution of 4-(2,6,6-trimethylcyclohex-1-en-1-yl)but-3-en-2-one (10.0 g, 52.0 mmol, 1.0 equiv) in THF (40 mL) was added dropwise at 0° C. (the temperature rose to 11° C.). After the addition was completed, the cooling bath was removed and the reaction mixture was stirred for 1 hour at room temperature and refluxed for 1.5 hours. Then 63% aq. sulfuric acid (12.0 mL, 139 mmol, 2.7 equiv) was added dropwise at 3° C. to the reaction mixture. After stirring for 1 hour at room temperature, the reaction mixture was added to 120 mL of water for quenching and extracted with MTBE (150 mL). The organic layer was washed once with 2M NaOH (100 mL), water (150 mL) and brine (150 mL). The organic layer was dried over MgSO₄, filtered and the solvent was removed under reduced pressure. The crude was filtered over silica gel and eluted with 100% heptane to give 2-(6-bromo-3-methylhexa-1,3-dien-1-yl)-1,3,3-trimethylcyclohex-1-ene (6.64 g, 43% yield, mixture of 4 isomers 74:16:2:1, 93% purity) as a yellow liquid.

GC/MS (EI) major isomer: m/z (%): 296 (10, [M]⁺·), 133 (45), 119 (100), 105 (46), 91 (45), 41 (30).

### b) 3-ethoxy-4-((4-methyl-6-(2,6,6-trimethylcyclohex-1-en-1-yl)hexa-3,5-dien-1-yl)oxy)benzaldehyde

The compound was obtained from 3-ethoxy-4-hydroxybenzaldehyde (1.86 g, 11.2 mmol, 1.0 equiv) and 2-(6-bromo-3-methylhexa-1,3-dien-1-yl)-1,3,3-trimethylcyclohex-1-ene (3.32 g, 11.2 mmol, 1.0 equiv) according to the procedure of example 42 as a yellow liquid (0.78 g, 18% yield, 4 isomers 81:13:6 overlapping in GC-MS).

¹H NMR (400 MHz, CDCl₃, complex mixture of isomers): δ 9.84 (s, 1H), 7.47-7.37 (m, 2H), 7.01-6.92 (m, 1H), 6.55-6.37 (m, 0.4H), 6.26-5.98 (m, 1.6H), 5.69 (td, J=4.4, 16.1 Hz, 0.1H), 5.49 (dt, J=1.1, 7.5 Hz, 0.6H), 5.40 (t, J=7.2 Hz, 0.3H), 4.18-4.07 (m, 4H), 2.79-2.72 (m, 1.6H), 2.54-2.25 (m, 0.4H), 2.11-1.40 (m, 15H), 1.11-0.81 (m, 6H) ppm. ¹³C NMR (101 MHz, CDCl₃, complex mixture of isomers): δ 190.9, 154.5, 154.3, 154.2, 149.1, 149.1, 138.0, 137.6, 137.3, 136.9, 135.4, 130.0, 129.7, 129.0, 128.6, 128.4, 126.5, 125.5, 124.7, 122.7, 119.6, 111.9, 111.8, 111.7, 110.9, 68.7, 68.3, 64.6, 40.6, 39.5, 39.4, 36.6, 34.2, 34.1, 32.9, 31.9, 29.0, 28.9, 28.9, 28.7, 28.6, 28.2, 27.3, 24.6, 22.8, 22.7, 21.7, 21.6, 20.5, 19.2, 19.2, 14.7, 14.7, 14.1, 12.5 ppm.

Odour description (1% solution in EtOH on paper blotter, 24 h): powdery, vanilla, fruity, woody, ionone beta.

### Example 84: 4-((5,9-dimethyldeca-3,8-dien-1-yl)oxy)-3-ethoxybenzaldehyde

The compound was obtained from 3-ethoxy-4-hydroxybenzaldehyde (2.00 g, 12.0 mmol, 1.0 equiv) and 5,9-dimethyldeca-3,8-dien-1-ol (2.20 g, 12.0 mmol, 1.0 equiv, prepared following a procedure by Gruenanger, C. U.; Breit, B. Angew. Chem. Int. Ed. 2008, 47, 7346-7349) according to the procedure of example 1 as a colorless liquid (1.78 g, 45% yield).

¹H NMR (400 MHz, CDCl₃): δ 9.84 (s, 1H), 7.42 (dd, J=2.0, 8.1 Hz, 1H), 7.40 (d, J=1.8 Hz, 1H), 6.97 (d, J=8.2 Hz, 1H), 5.49-5.41 (m, 2H), 5.09 (tdt, J=1.4, 2.8, 7.2 Hz, 1H), 4.18-4.07 (m, 4H), 2.62-2.52 (m, 2H), 2.11 (m_{c}, 1H), 2.00-1.91 (m, 2H), 1.68 (d, J=1.2 Hz, 3H), 1.60-1.55 (m, 3H), 1.47 (t, J=7.0 Hz, 3H), 1.36-1.23 (m, 2H), 0.98 (d, J=6.7 Hz, 3H) ppm. ¹³C NMR (101 MHz, CDCl₃, mixture of isomers): δ 190.9, 154.3, 149.1, 139.6, 131.2, 129.9, 126.5, 124.6, 123.1, 111.8, 110.9, 68.8, 64.5, 37.0, 36.4, 32.3, 25.8, 25.7, 20.6, 17.6, 14.6 ppm.

Odour description (1% solution in EtOH on paper blotter, 24 h): powdery, vanilla, chocolate milk, slightly fresh, fatty, watery, melon.

### Example 85: 3-ethoxy-4-((6-phenylhex-3-en-1-yl)oxy)benzaldehyde

The compound was obtained from 3-ethoxy-4-hydroxybenzaldehyde (2.69 g, 12.0 mmol, 1.0 equiv) and (6-bromohex-3-en-1-yl)benzene (3.87 g, 16.2 mmol, 1.0 equiv, prepared according to Matsubara, Hiroshi; et al. Japan, JP2017014124 A, contains also (3-bromo-3-cyclopropylpropyl)benzene) according to the procedure of example 42 as a colorless liquid (2.0 g, 38% yield, 61% pure, E isomer along with 39% of 4-(1-cyclopropyl-3-phenylpropoxy)-3-ethoxybenzaldehyde).

¹H NMR (400 MHz, CDCl₃, signals for the mixture): δ 9.85 (s, 2H), 7.46-7.41 (m, 3H), 7.37 (dd, J=2.0, 8.1 Hz, 1H), 7.31-7.16 (m, 11H), 6.96 (dd, J=3.4, 8.1 Hz, 2H), 5.71-5.59 (m, 1H), 5.57-5.46 (m, 1H), 4.18-4.05 (m, 6H), 3.86 (dt, J=5.1, 7.3 Hz, 1H), 2.93-2.78 (m, 2H), 2.74-2.66 (m, 2H), 2.57 (dq, J=0.9, 6.9 Hz, 2H), 2.39-2.33 (m, 2H), 2.25-2.04 (m, 2H), 1.47 (q, J=6.8 Hz, 6H), 1.18 (tq, J=5.1, 8.1 Hz, 1H), 0.61-0.50 (m, 2H), 0.39-0.22 (m, 2H) ppm. ¹³C NMR (101 MHz, CDCl₃, signals for the mixture): δ 190.9, 190.9, 154.3, 154.1, 150.5, 149.1, 141.8, 132.8, 130.6, 130.0, 128.4, 128.4, 128.2, 126.5, 125.9, 125.9, 125.8, 125.6, 116.7, 111.9, 111.7, 111.0, 83.2, 68.7, 64.6, 64.5, 36.6, 35.8, 34.5, 32.2, 31.5, 15.2, 14.7, 14.7, 3.5, 2.2 ppm.

Odour description (1% solution in EtOH on paper blotter, 24 h): powdery, vanilla, creamy, balsamic, cinnamic).

### Example 86: 3-ethoxy-4-((6-(4-isobutylphenyl)hex-3-en-1-yl)oxy)benzaldehyde

### a) 1-cyclopropyl-3-(4-isobutylphenyl)prop-2-en-1-one

Sodium hydroxide (24.0 g, 594 mmol, 5.0 equiv) was added to a mixture of ethanol (90 mL) and water (60 mL) and stirred for a few minutes (the temperature rose to 70° C.). Then, cyclopropyl methyl ketone (10.0 g, 119 mmol, 1.0 equiv) was added dropwise to the suspension of base followed by 4-isobutylbenzaldehyde (19.3 g, 119 mmol, 1.0 equiv. After stirring for 3 hours at 60° C., the reaction mixture was added to ice cold water (120 mL) and extracted with MTBE (2×150 mL). The organic layers were washed with 2M HCl (150 mL), water (150 mL) and brine (150 mL). The combined organic layers were dried over MgSO₄, filtered and concentrated under reduced pressure to give crude 1-cyclopropyl-3-(4-isobutylphenyl)prop-2-en-1-one (25.8 g, yellow liquid).

### b) 1-cyclopropyl-3-(4-isobutylphenyl)propan-1-one

To a solution of crude 1-cyclopropyl-3-(4-isobutylphenyl)prop-2-en-1-one (10.0 g) in MeOH (20 mL) was added Raney-Nickel (0.5 g, 5% wt/wt) in an autoclave and the reaction mixture was pressurized with hydrogen (30 bar) at 100° C. for 5 hours. The suspension was filtered and concentrated under reduced pressure to give crude 1-cyclopropyl-3-(4-isobutylphenyl)propan-1-one (9.9 g) as a colorless liquid.

### c) 1-cyclopropyl-3-(4-isobutylphenyl)propan-1-ol

LiAlH₄ (1.20 g, 0.8 equiv) was suspended in Et₂O (90 mL) and a solution of crude 1-cyclopropyl-3-(4-isobutylphenyl)propan-1-one (9.9 g) in Et₂O (30 mL) was added dropwise at 0° C. After stirring for 2 hours at room temperature, 2M aq. NaOH (9.9 mL) was added dropwise and slowly. After stirring for 20 minutes, one spatula of MgSO₄ was added. After stirring for another 20 minutes, the reaction mixture was filtered and the solvent removed under reduced pressure to give crude 1-cyclopropyl-3-(4-isobutylphenyl)propan-1-ol (9.16 g) as a colorless liquid.

### d) 1-(6-bromohex-3-en-1-yl)-4-isobutylbenzene

To a round-bottomed flask containing crude 1-cyclopropyl-3-(4-isobutylphenyl)propan-1-ol (8.48 g) was added dropwise at 15° C. 48% aq. HBr (12.3 g, 2.0 equiv). After stirring for 3 hours at room temperature, more 48% aq. HBr (2.0 g) was added and stirring was continued for 1 hour. The reaction mixture was quenched with ice-cold 2M aq. NaOH and extracted with MTBE (50 mL). The organic layer was washed with water (50 mL) and brine (50 mL). The combined organic extracts were dried over MgSO₄, filtered and concentrated under reduced pressure to give crude 1-(6-bromohex-3-en-1-yl)-4-isobutylbenzene (9.4 g) as a yellow liquid, which was used in the next step without further purification.

### e) 3-ethoxy-4-((6-(4-isobutylphenyl)hex-3-en-1-yl)oxy)benzaldehyde

The compound was obtained from 3-ethoxy-4-hydroxybenzaldehyde (2.50 g, 15.0 mmol, 1.0 equiv) and crude 1-(6-bromohex-3-en-1-yl)-4-isobutylbenzene (4.40 g, 14.9 mmol, 1.0 equiv) according to the procedure of example 42 as a colorless liquid (1.14 g, 20% yield, 15% pure along with 85% of 4-(1-cyclopropyl-3-(4-isobutylphenyl)propoxy)-3-ethoxybenzaldehyde). Characteristic signals for 3-ethoxy-4-((6-(4-isobutylphenyl)hex-3-en-1-yl)oxy)benzaldehyde: ¹H NMR (400 MHz, CDCl₃): δ 5.74-5.58 (m, 1H), 5.56-5.45 (m, 1H). Characteristic signals for 4-(1-cyclopropyl-3-(4-isobutylphenyl)propoxy)-3-ethoxybenzaldehyde: ¹³C NMR (101 MHz, CDCl₃): δ 191.0, 154.2, 150.5, 139.2, 138.9, 130.6, 129.1, 129.0, 128.1, 126.0, 116.6, 111.7, 83.2 (CH—O), 64.5, 45.0, 36.7, 31.1, 30.2, 22.4, 15.2, 14.7, 3.4, 2.3 ppm.

Odour description for the mixture (1% solution in EtOH on paper blotter, 24 h): powdery, vanilla, creamy, floral, muguet.

### Example 87: 3-ethoxy-4-(3-(2-(2-(4-methylcyclohex-3-en-1-yl)propyl)cyclopentylidene) propoxy)benzaldehyde

### a) 4-(1-(2-(3-bromopropylidene)cyclopentyl)propan-2-yl)-1-methylcyclohex-1-ene

Magnesium turnings (1.10 g, 44 mmol, 1.2 equiv) were added to a 350 ml-sulfonation flask and overlayed with THF (10 mL). Then 10% of a solution of bromocyclopropane (5.3 g, 44 mmol, 1.2 equiv) in THF (40 mL) were added. Afterwards the Grignard was started with a heating gun and a few iodine crystals. Then the remaining THF solution of bromocyclopropane was added dropwise. After the addition was completed, the reaction mixture was stirred for 1 hour at reflux. Then, a solution of 2-(2-(4-methylcyclohex-3-en-1-yl)propyl)cyclopentan-1-one (8.0 g, 36 mmol, 1.0 equiv) in THF (40 mL) was added dropwise at 0° C. (the temperature rose to 10° C.). After the addition was completed, the cooling bath was removed and the reaction mixture was stirred for 1 hour at room temperature and refluxed for 1.5 hours. Then 63% aq. sulfuric acid (8.0 mL, 98 mmol, 2.7 equiv) was added dropwise at 3° C. to the reaction mixture (the temperature rose to 18° C.). After stirring for 1 hour at room temperature, the reaction mixture was added to 120 mL of water for quenching and extracted with MTBE (150 mL). The organic layer was washed once with water (2×150 mL) and brine (150 mL). The organic layer was dried over MgSO₄, filtered and the solvent was removed under reduced pressure. The crude was filtered over silica gel and eluted with 100% heptane to give 4-(1-(2-(3-bromopropylidene)cyclopentyl)propan-2-yl)-1-methylcyclohex-1-ene (1.93 g, 16% yield, 63% pure) as a yellow liquid.

GC/MS (EI): m/z (%): 244 (12, [M]⁺·-HBr), 121 (100), 93 (94), 79 (80), 67 (40), 55 (35).

### b) 3-ethoxy-4-(3-(2-(2-(4-methylcyclohex-3-en-1-yl)propyl)cyclopentylidene) propoxy)benzaldehyde

The compound was obtained from 3-ethoxy-4-hydroxybenzaldehyde (0.51 g, 3.1 mmol, 1.0 equiv) and 4-(1-(2-(3-bromopropylidene)cyclopentyl)propan-2-yl)-1-methylcyclohex-1-ene (1.0 g, 3.1 mmol, 1.0 equiv) according to the procedure of example 42 as a colorless liquid (0.29 g, 23% yield, 4 isomers 59:29:4:3).

¹H NMR (400 MHz, CDCl₃, complex mixture of isomers): δ 9.77 (s, 1H), 7.37-7.31 (m, 2H), 6.93-6.85 (m, 1H), 5.31 (br s, 1H), 5.24-5.07 (m, 1H), 4.18-3.91 (m, 4H), 3.41 (q, J=6.9 Hz, 1H), 2.62-0.90 (m, 24H), 0.89-0.62 (m, 3H) ppm. ¹³C NMR (101 MHz, CDCl₃, complex mixture of isomers): δ 190.5, 154.3, 154.2, 152.7, 150.8, 150.4, 148.9, 146.8, 135.9, 135.2, 134.6, 133.6, 133.6, 133.5, 129.7, 129.7, 129.6, 129.6, 126.2, 124.0, 123.2, 121.0, 120.9, 120.8, 120.8, 120.7, 115.7, 113.8, 113.8, 113.5, 112.4, 111.5, 111.5, 111.4, 111.3, 111.2, 110.6, 110.6, 110.4, 68.5, 68.4, 68.3, 68.2, 68.2, 65.6, 64.2, 64.2, 56.3, 51.9, 50.5, 48.6, 45.8, 45.7, 44.2, 43.6, 42.2, 42.0, 41.8, 41.8, 39.5, 39.5, 39.3, 39.3, 39.2, 39.0, 38.9, 38.6, 38.3, 38.0, 36.9, 36.5, 35.8, 35.6, 35.4, 35.3, 35.1, 35.4, 35.1, 34.9, 34.6, 33.1, 33.1, 32.9, 32.6, 32.3, 30.9, 30.7, 30.7, 30.3, 30.3, 29.9, 29.8, 29.6, 29.4, 29.2, 29.2, 28.9, 28.8, 28.7, 27.9, 27.9, 27.5, 27.3, 27.1, 26.8, 26.5, 25.9, 25.8, 25.6, 25.0, 24.9, 24.3, 23.8, 23.8, 23.7, 23.7, 23.5, 23.4, 23.2, 22.3, 21.7, 20.3, 19.8, 16.5, 16.1, 15.7, 15.7, 15.4, 15.1, 14.6, 14.5 ppm. Odour description (1% solution in EtOH on paper blotter, 24 h): green, fatty, creamy, warm milk, fatty, powdery, vanilla, fruity, peachy.

### Example 88: 4-((4-cyclopentylpent-3-en-1-yl)oxy)-3-ethoxybenzaldehyde

### a) 1-cyclopentyl-1-cyclopropylethan-1-ol

Magnesium turnings (1.04 g, 42.8 mmol, 1.2 equiv) were added to a 100 mL-sulfonation flask and overlayed with THF (5 mL). Then 10% of a solution of bromocyclopentane (6.38 g, 42.8 mmol, 1.2 equiv) in THF (20 mL) were added. After the Grignard reaction had been started, the remaining THF solution of bromocyclopentane was added dropwise. The reaction mixture was refluxed for 1 hour. Then, a solution of 1-cyclopropylethan-1-one (3.00 g, 35.7 mmol, 1.0 equiv) in THF (20 mL) was added dropwise at 0° C. After stirring for 3 hours at room temperature, the reaction mixture was quenched with water (30 mL) and extracted with MTBE (40 mL). The organic layer was washed water (2×40 mL) and brine (40 mL). The organic layer was dried over MgSO₄, filtered and the solvent was removed under reduced pressure to give crude 1-cyclopentyl-1-cyclopropylethan-1-ol (2.88 g) as a colorless liquid.

### b) (5-bromopent-2-en-2-yl)cyclopentane

To a round-bottomed flask containing 1-cyclopentyl-1-cyclopropylethan-1-ol (2.88 g, 18.7 mmol) was added dropwise at 15° C. 48% aq. HBr (4.09 g, 24.3 mmol, 1.3 equiv). After stirring for 1 hour at room temperature, the reaction mixture was added onto ice cold 2M aq. NaOH (10 mL) and extracted with MTBE (20 mL). The organic layer was washed water (20 mL) and brine (20 mL). The organic layer was dried over MgSO₄, filtered and the solvent was removed under reduced pressure to give crude (5-bromopent-2-en-2-yl)cyclopentane (3.08 g, 59% pure by GC) as a brown liquid.

### c) 4-((4-cyclopentylpent-3-en-1-yl)oxy)-3-ethoxybenzaldehyde

The compound was obtained from 3-ethoxy-4-hydroxybenzaldehyde (2.36 g, 14.2 mmol, 1.0 equiv) and crude (5-bromopent-2-en-2-yl)cyclopentane (3.08 g, 14.2 mmol, 1.0 equiv) according to the procedure of example 42 as a yellow liquid (0.20 g, 5% yield, 2 isomers 86:13).

¹H NMR (400 MHz, CDCl₃, mixture of isomers): δ 9.75 (s, 1H), 7.34 (d, J=8.1 Hz, 1H), 7.32 (s, 1H), 6.89 (d, J=7.7 Hz, 1H), 5.21-5.11 (m, 1H), 4.08-3.94 (m, 4H), 2.57-2.46 (m, 2H), 2.37-2.28 (m, 1H), 1.69-1.44 (m, 9H), 1.42-1.25 (m, 5H) ppm. ¹³C NMR (101 MHz, CDCl₃, mixture of isomers): δ 190.5, 154.2, 148.9, 141.2, 140.8, 129.7, 126.2, 119.2, 116.6, 111.5, 111.5, 110.7, 110.5, 68.6, 68.3, 64.2, 49.1, 48.7, 40.5, 30.7, 30.4, 27.6, 27.2, 26.7, 25.5, 25.0, 18.9, 14.4, 14.2 ppm. GC/MS (EI, major): m/z (%): 302 (1, [M]⁺·), 137 (25), 95 (100), 81 (73), 67 (28), 55 (30), 41 (28).

Odour description (1% solution in EtOH on paper blotter, 24 h): powdery, creamy, vanilla.

### Example 89: 3-ethoxy-4-((4-methyldec-3-en-1-yl)oxy)benzaldehyde

### a) 1-bromo-4-methyldec-3-ene

Magnesium turnings (1.04 g, 42.8 mmol, 1.2 equiv) were added to a 100 mL-sulfonation flask and overlayed with THF (5 mL). Then 10% of a solution of 1-bromohexane (7.06 g, 42.8 mmol, 1.2 equiv) in THF (20 mL) were added. After the Grignard reaction had been started, the remaining THF solution of 1-bromohexane was added dropwise. After the addition was completed, the reaction mixture was stirred for 1 hour at reflux. Then, a solution of 1-cyclopropylethan-1-one (3.00 g, 35.7 mmol, 1.0 equiv) in THF (20 mL) was added dropwise at 0° C. After the addition was completed, the cooling bath was removed and the reaction mixture was stirred for 2 hours at room temperature. 63% aq. sulfuric acid (15 mL, 96 mmol, 2.7 equiv) was added dropwise at 0° C. to the reaction mixture. After stirring for 16 hours at room temperature, the reaction mixture was quenched with water (40 mL) and extracted with MTBE (40 mL). The organic layer was washed water (2×40 mL) and brine (40 mL). The organic layer was dried over MgSO₄, filtered and the solvent was removed under reduced pressure to give crude 1-bromo-4-methyldec-3-ene (6.06 g) as a yellow liquid.

### b): 3-ethoxy-4-((4-methyldec-3-en-1-yl)oxy)benzaldehyde

The compound was obtained from 3-ethoxy-4-hydroxybenzaldehyde (4.32 g, 26.0 mmol, 1.0 equiv) and crude 1-bromo-4-methyldec-3-ene (6.06 g, 26.0 mmol, 1.1 equiv) according to the procedure of example 42 as an orange liquid (3.22 g, 39% yield, 2 isomers 89:11, 85% purity).

¹³C NMR (101 MHz, CDCl₃, major): δ 190.9, 154.4, 149.1, 139.1, 129.9, 126.6, 118.4, 111.7, 110.9, 68.7, 64.5, 39.7, 31.8, 28.9, 27.9, 27.8, 22.6, 16.1, 14.7, 14.1 ppm. GC/MS (EI, major): m/z (%): 318 (3, [M]⁺·), 111 (31), 97 (100), 83 (71), 69 (75), 55 (62), 41 (37). GC/MS (EI, minor): m/z (%): 318 (1, [M]⁺·), 111 (36), 97 (100), 83 (74), 69 (79), 55 (63), 41 (32).

Odour description (1% solution in EtOH on paper blotter, 24 h): powdery, vanilla, creamy.

### Example 90: 3-ethoxy-4-((4-methyldodec-3-en-1-yl)oxy)benzaldehyde

### a) 1-bromo-4-methyldodec-3-ene

Magnesium turnings (1.04 g, 42.8 mmol, 1.2 equiv) were added to a 100 mL-sulfonation flask and overlayed with THF (5 mL). Then 10% of a solution of 1-bromooctane (8.27 g, 42.8 mmol, 1.2 equiv) in THF (20 mL) were added. After the Grignard reaction had been started, the remaining THF solution of 1-bromooctane was added dropwise. The reaction mixture was refluxed for 1 hour. Then, a solution of 1-cyclopropylethan-1-one (3.00 g, 35.7 mmol, 1.0 equiv) in THF (20 mL) was added dropwise at 5° C. After the addition was completed, the cooling bath was removed and the reaction mixture was stirred for 2 hours at room temperature. 63% aq. sulfuric acid (8.15 mL, 96 mmol, 2.7 equiv) was added dropwise at 0° C. to the reaction. After stirring for 16 hours at room temperature, the reaction mixture was quenched with water (40 mL) and extracted with MTBE (40 mL). The organic layer was washed water (2×40 mL) and brine (40 mL). The organic layer was dried over MgSO₄, filtered and the solvent was removed under reduced pressure to give crude 1-bromo-4-methyldodec-3-ene (7.47 g) as a yellow liquid.

### b) 3-ethoxy-4-((4-methyldodec-3-en-1-yl)oxy)benzaldehyde

The compound was obtained from 3-ethoxy-4-hydroxybenzaldehyde (4.75 g, 28.6 mmol, 1.0 equiv) and crude 1-bromo-4-methyldodec-3-ene (7.47 g, 28.6 mmol, 1.1 equiv) according to the procedure of example 42 as a yellow liquid (2.58 g, 26% yield, 2 isomers 94:6).

¹H NMR (400 MHz, CDCl₃, mixture of isomers): δ 9.83 (s, 1H), 7.43-7.39 (m, 2H), 6.96 (d, J=8.1 Hz, 1H), 5.20 (ddd, J=1.2, 6.1, 8.6 Hz, 1H), 4.18-4.12 (m, 2H), 4.08-4.03 (m, 2H), 2.58 (q, J=7.3 Hz, 2H), 2.08-1.98 (m, 2H), 1.74-1.64 (m, 3H), 1.49-1.45 (m, 3H), 1.43-1.35 (m, 2H), 1.33-1.16 (m, 10H), 0.90-0.85 (m, 3H) ppm. ¹³C NMR (101 MHz, CDCl₃, mixture of isomers: δ 190.9, 154.4, 154.3, 149.1, 139.2, 139.0, 129.9, 129.9, 126.5, 119.0, 118.3, 111.7, 110.8, 68.8, 68.6, 64.5, 39.7, 32.0, 31.9, 29.6, 29.5, 29.5, 29.3, 29.3, 28.1, 27.9, 27.8, 27.7, 23.5, 22.6, 16.1, 14.6, 14.1 ppm. GC/MS (EI, major): m/z (%): 346 (2, [M]⁺·), 111 (47), 97 (95), 83 (86), 69 (100), 55 (67), 41 (47). GC/MS (EI, minor): m/z (%): 346 (2, [M]⁺·), 111 (46), 97 (100), 83 (90), 69 (99), 55 (78), 41 (42).

Odour description (1% solution in EtOH on paper blotter, 24 h): powdery, vanilla, creamy, slightly green, sesame seed, sharp, technical.

### Example 91: 3-ethoxy-4-(tridec-1-en-4-yloxy)benzaldehyde

The compound was obtained from tridec-1-en-4-ol (5.00 g, 25.2 mmol), ethyl vanillin (3-ethoxy-4-hydroxybenzaldehyde) (8.12 g, 47.9 mmol), triphenylphosphine (7.93 g, 30.2 mmol) and DIAD (diisopropyl azodicarboxylate) (6.24 g, 30.2 mmol) according to the procedure of example 1 as a colorless oil (5.22 g, 60% yield).

¹H NMR (400 MHz, CDCl3, 298 K) δ (ppm)=9.83 (s, 1H), 7.45-7.36 (m, 2H), 6.98 (d, J=8.8 Hz, 1H), 5.87 (tdd, J=7.1, 10.1, 17.1 Hz, 1H), 5.19-5.05 (m, 2H), 4.38 (quin, J=5.9 Hz, 1H), 4.13 (q, J=7.0 Hz, 2H), 2.56-2.38 (m, 2H), 1.82-1.63 (m, 2H), 1.50-1.20 (m, 18H), 0.91-0.83 (m, 3H). ¹³C NMR (101 MHz, CDCl3, 298 K) δ (ppm)=191.0, 154.1, 150.1, 133.9, 130.1, 126.3, 117.7, 114.7, 111.7, 79.5, 64.6, 38.3, 33.7, 31.9, 29.6, 29.5, 29.5, 29.3, 25.3, 22.7, 14.7, 14.1. MS (EI, 70 eV): 346 (4, [M]+·), 305 (1), 166 (100), 138 (44), 55 (16), 41 (17). Odour description (1% solution in EtOH on paper blotter, 24 h): powdery, vanilla, aldehydic, fruity, mandarin.

1. A method of generating a phenolic fragment compound HX which is a compound of formula (II) 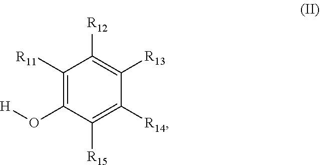
the method comprising exposing a compound of formula (I) to ambient air in the presence or absence of light 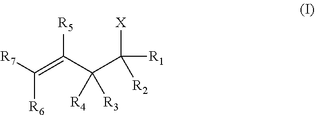
wherein
R₁-R₇ is independently selected from the group consisting of H, Me, linear C2-C20 hydrocarbon groups and branched C2-C20 hydrocarbon groups, wherein the linear and branched C2-C20 hydrocarbon groups independently bear up to 4 heteroatoms selected from the group consisting of O, S, and N, and bearing up to three double bonds or two triple bonds and bear up to three rings, phenyl, benzyl, substituted aryl or hetero-aryl units, 1- or 2-naphthyl,
or R₆ and R₇ form together a double bond to a carbon atom, which is bearing one or two C1-C10 alkyl or C2-C10 alkenyl substituents, while the other substituents have the same meaning as previously defined;
or R₁ and R₆, R₁ and R₃, R₁ and R₅, R₃ and R₅, R₃ and R₆, R₅ and R₇, R₆ and R₇, R₁ and R₂, or R₃ and R₄, form together with the carbon atoms of the chain they are attached to a 5 or 6 membered ring bearing up to 2 heteroatoms selected from the group consisting of O, S and N, that is optionally substituted with an alkyl chain, while the other substituents have the same meaning as previously defined;
and the sum of all carbon atoms of R₁-R₇ is at least 5;
or R₇ is —CR′₃R′₄—CR′₁R′₂—X′, wherein R′₁, R′₂, R′₃, R′₄, and X′ are selected from the corresponding groups of R₁, R₂, R₃, R₄, and X, while the other substituents have the same meaning as previously defined, and the sum of all carbon atoms of R₁-R₆ and R′₁-R′₄ is at least 5 or more;
and wherein X is representing the following fragment 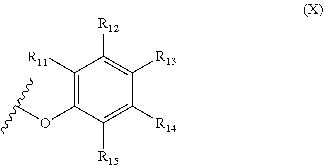
wherein
R₁₁-R₁₅ is independently selected from the group consisting of H, linear or branched C1-C5 alkyl, linear or branched C2-C5 alkenyl, linear or branched C2-C5 alkynyl, OH, C1-C4-alkoxy, CHO, C(O)Me, C(O)Et, hexanoyl, heptanoyl, octanoyl, propanoyl, 3-phenylpropanoyl, 5-methylhexanoyl, (CH₂)₂C(O)CH₃, (4-methyl-3,6-dihydro-2H-pyran-2-yl), CH₂—O-Me, CH₂—O-Et, CH₂—O-iPr, CH₂—OH, Cl, Br, phenyl, and CH(OR₁₆)(OR₁₇) wherein R₁₆ and R₁₇ are independently selected from the group consisting of C1-C5 alkyl, R₁₆ and R₁₇ form together with the carbon atoms of the chain they are attached to a 5 or 6 membered ring, C═N—OR₁₈ wherein R₁₈ is C1-C5 alkyl, and CO₂R₁₉ wherein R₁₉ is selected from the group consisting of linear or branched C1-C8 alkyl, linear or branched C2-C8 alkenyl, linear or branched C2-C8 alkynyl, cycloalkyl and aryl;
or R₁₁ and R₁₂ or R₁₂ and R₁₃ may form together with the carbon atoms they are attached to C1-C5 alkyl substituted or unsubstituted, saturated or unsaturated 5 and 6 membered rings containing C, O and/or N atoms.

2. The method according to claim 1, wherein
R₁-R₇ is independently selected from the group consisting of H, Me, C2-C20 branched or linear alkyl or alkenyl or alkynyl, cycloalkyl, cycloalkenyl, phenyl, benzyl, phenylethyl or substituted phenylethyl, substituted aryl or hetero-aryl units, 1- or 2-naphthyl, alkoxycarbonylates (—O(C═O)—R), alkylcarboxylates (—(C═O)—O—R), ethers, aldehydes, ketones, and alcohols;
or R₆ and R₇ form together a double bond to a carbon atom, which is bearing one or two C1-C10 alkyl or C2-C10 alkenyl substituents, while the other substituents have the same meaning as previously defined;
or R₁ and R₆, R₁ and R₃, R₁ and R₅, R₃ and R₅, R₃ and R₆, R₅ and R₇, R₆ and R₇, R₁ and R₂, or R₃ and R₄, form together with the carbon atoms of the chain they are attached to a 5 or 6 membered ring bearing up to 2 heteroatoms selected from the group consisting of O, S and N, that is optionally substituted with an alkyl chain, while the other substituents have the same meaning as previously defined;
and the sum of all carbon atoms of R₁-R₇ is at least 5;
or R₇ is —CR′₃R′₄—CR′₁R′₂—X′, wherein R′₁, R′₂, R′₃, R′₄, and X′ are selected from the corresponding groups of R₁, R₂, R₃, R₄, and X, while the other substituents have the same meaning as previously defined, and the sum of all carbon atoms of R₁-R₆ and R′₁-R′₄ is at least 5;
and wherein X is representing the following fragment 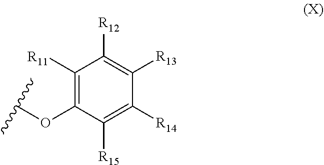
wherein
R₁₁ is selected from the group consisting of H, C1-C5 alkyl, OH, OMe, OEt, O-n-Pr, O-iso-Pr, hexanoyl, heptanoyl, octanoyl, propanoyl, 3-phenylpropanoyl, 5-methylhexanoyl, Cl, Br, phenyl, and CO₂R₁₉, wherein R₁₉ is selected from the group consisting of H, Me, Et, 2-methylpropyl, hexan-2-yl, 2-isopropoxyethyl, 3-methylbut-2-en-1-yl, n-pentyl, hexyl, benzyl, cyclohexyl, cis-3-hexenyl, 2-ethylhexyl, 3-methylhex-2-en-1-yl, 2-hept-4-enyl, 1-(3-methyl-2-hexenyl), (CH₂)₂-phenyl, para-tolyl;
R₁₂ is selected from the group consisting of H, OH, C1-C5 alkyl, C2-C5 alkenyl, and C1-C5 alkoxy;
R₁₃ is selected from the group consisting of H, branched or linear C1-C5 alkyl, branched or linear C2-C5 alkenyl, CHO, CO₂H, CO₂Me, CO₂Et, C(O)Me, C(O)Et, (CH₂)₂C(O)CH₃;
(4-methyl-3,6-dihydro-2H-pyran-2-yl), CH₂—O-Me, CH₂—O-Et, CH₂—O-iPr, CH₂—OH, Cl, propanoyl, and CO₂R₁₉, wherein R₁₉ is selected from the group consisting of H, Me, Et, 2-methylpropyl, hexan-2-yl, 2-isopropoxyethyl, 3-methylbut-2-en-1-yl, n-pentyl, hexyl, benzyl, cyclohexyl, cis-3-hexenyl, 2-ethylhexyl, 3-methylhex-2-en-1-yl, 2-hept-4-enyl, 1-(3-methyl-2-hexenyl), (CH₂)₂-phenyl, and para-tolyl;
R₁₄ is selected from the group consisting of H, OH, C1-C5 alkyl, C2-C5 alkenyl, and C1-C5 alkoxy;
R₁₅ is selected from the group consisting of H, C1-C5 alkyl, and Br;
or R₁₁ and R₁₂ or R₁₂ and R₁₃ may form together with the carbon atoms they are attached to C1-C5 alkyl substituted or unsubstituted, saturated or unsaturated 5 and 6 membered rings containing C, O and/or N atoms,
as precursor for generating a phenolic fragrant compound HX which is a compound of formula (II)

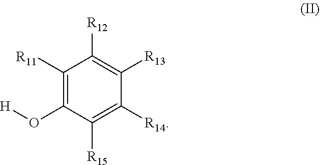

3. A compound of formula (I) 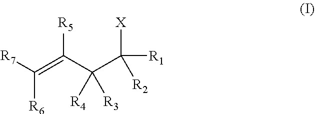
wherein
R₁-R₇ is independently selected from the group consisting of H, Me, linear C2-C20 hydrocarbon groups and branched C2-C20 hydrocarbon groups, wherein the linear and branched C2-C20 hydrocarbon groups independently bear up to 4 heteroatoms selected from the group consisting of O, S, and N, and bearing up to three double bonds or two triple bonds and bear up to three rings, phenyl, benzyl, substituted aryl or hetero-aryl units, 1- or 2-naphthyl,
or R₆ and R₇ form together a double bond to a carbon atom, which is bearing one or two C1-C10 alkyl or C2-C10 alkenyl substituents, while the other substituents have the same meaning as previously defined;
or R₁ and R₆, R₁ and R₃, R₁ and R₅, R₃ and R₅, R₃ and R₆, R₅ and R₇, R₆ and R₇, R₁ and R₂, or R₃ and R₄, form together with the carbon atoms of the chain they are attached to a 5 or 6 membered ring bearing up to 2 heteroatoms selected from the group consisting of O, S and N, that is optionally substituted with an alkyl chain, while the other substituents have the same meaning as previously defined;
and the sum of all carbon atoms of R₁-R₇ is at least 5 or more;
or R₇ is —CR′₃R′₄—CR′₁R′₂—X′, wherein R′₁, R′₂, R′₃, R′₄, and X′ are selected from the corresponding groups of R₁, R₂, R₃, R₄, and X, while the other substituents have the same meaning as previously defined, and the sum of all carbon atoms of R₁-R₆ and R′₁-R′₄ is at least 5 or more;
and wherein X is representing the following fragment 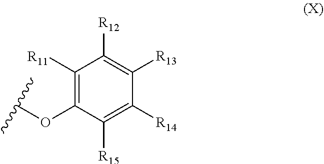
wherein
R₁₁-R₁₅ is independently selected from the group consisting of H, linear or branched C1-C5 alkyl, linear or branched C2-C5 alkenyl, linear or branched C2-C5 alkynyl, OH, C1-C4-alkoxy, CHO, C(O)Me, C(O)Et, hexanoyl, heptanoyl, octanoyl, propanoyl, 3-phenylpropanoyl, 5-methylhexanoyl, (CH₂)₂C(O)CH₃, (4-methyl-3,6-dihydro-2H-pyran-2-yl), CH₂—O-Me, CH₂—O-Et, CH₂—O-iPr, CH₂—OH, Cl, Br, phenyl, CH(OR₁₆)(OR₁₇) wherein R₁₆ and R₁₇ are independently selected from the group consisting of C1-C5 alkyl or R₁₆ and R₁₇ form together with the carbon atoms of the chain they are attached to a 5 or 6 membered ring, C═N—OR₁₃ wherein R₁₈ is C1-C5 alkyl, CO₂R₁₉ wherein R₁₉ is selected from the group consisting of linear or branched C1-C8 alkyl, linear or branched C2-C8 alkenyl, linear or branched C2-C8 alkynyl, cycloalkyl and aryl;
or R₁₁ and R₁₂ or R₁₂ and R₁₃ may form together with the carbon atoms they are attached to C1-C5 alkyl substituted or unsubstituted, saturated or unsaturated 5 and 6 membered rings containing C, O and/or N atoms.

4. The compound according to claim 3
wherein
R₁-R₇ is independently selected from the group consisting of H, Me, C2-C20 branched or linear alkyl or alkenyl or alkynyl, cycloalkyl, cycloalkenyl, phenyl, benzyl, phenylethyl or substituted phenylethyl, substituted aryl or hetero-aryl units, 1- or 2-naphthyl, alkoxycarbonylates (—RO(C═O)—R′), alkylcarboxylates (—R(C═O)—O—R′), ethers, aldehydes, ketones, and alcohols;
or R₆ and R₇ form together a double bond to a carbon atom, which is bearing one or two C1-C10 alkyl or C2-C10 alkenyl substituents, while the other substituents have the same meaning as previously defined;
or R₁ and R₆, R₁ and R₃, R₁ and R₅, R₃ and R₅, R₃ and R₆, R₅ and R₇, R₆ and R₇, R₁ and R₂, or R₃ and R₄, form together with the carbon atoms of the chain they are attached to a 5 or 6 membered ring bearing up to 2 heteroatoms selected from the group consisting of O, S and N, that is optionally substituted with an alkyl chain, while the other substituents have the same meaning as previously defined;
and the sum of all carbon atoms of R₁-R₇ is at least 5;
or R₇ is —CR′₃R′₄—CR′₁R′₂—X′, wherein R′₁, R′₂, R′₃, R′₄, and X′ are selected from the corresponding groups of R₁, R₂, R₃, R₄, and X, while the other substituents have the same meaning as previously defined, and the sum of all carbon atoms of R₁-R₆ and R′₁-R′₄ is at least 5;
and wherein X is representing the following fragment 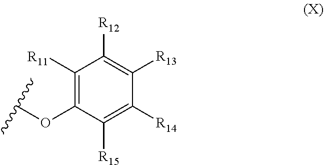
wherein
R₁₁ is selected from the group consisting of H, C1-C5 alkyl, OH, OMe, OEt, O-n-Pr, O-iso-Pr, hexanoyl, heptanoyl, octanoyl, propanoyl, 3-phenylpropanoyl, 5-methylhexanoyl, Cl, Br, phenyl, and CO₂R₁₉, wherein R₁₉ is selected from the group consisting of H, Me, Et, 2-methylpropyl, hexan-2-yl, 2-isopropoxyethyl, 3-methylbut-2-en-1-yl, n-pentyl, hexyl, benzyl, cyclohexyl, cis-3-hexenyl, 2-ethylhexyl, 3-methylhex-2-en-1-yl, 2-hept-4-enyl, 1-(3-methyl-2-hexenyl), (CH₂)₂-phenyl, and para-tolyl;
R₁₂ is selected from the group consisting of H, OH, C1-C5 alkyl, C2-C5 alkenyl, and C1-C5 alkoxy;
R₁₃ is selected from the group consisting of H, branched or linear C1-C5 alkyl, branched or linear C2-C5 alkenyl, CHO, CO₂H, CO₂Me, CO₂Et, C(O)Me, C(O)Et, (CH₂)₂C(O)CH₃;
(4-methyl-3,6-dihydro-2H-pyran-2-yl), CH₂—O-Me, CH₂—O-Et, CH₂—O-iPr, CH₂—OH, Cl, propanoyl, and CO₂R₁₉, wherein R₁₉ is selected from the group consisting of H, Me, Et, 2-methylpropyl, hexan-2-yl, 2-isopropoxyethyl, 3-methylbut-2-en-1-yl, n-pentyl, hexyl, benzyl, cyclohexyl, cis-3-hexenyl, 2-ethylhexyl, 3-methylhex-2-en-1-yl, 2-hept-4-enyl, 1-(3-methyl-2-hexenyl), (CH₂)₂-phenyl, and para-tolyl;
R₁₄ is selected from the group consisting of H, OH, C1-C5 alkyl, C2-C5 alkenyl, and C1-C5 alkoxy;
R₁₅ is selected from the group consisting of H, C1-C5 alkyl, and Br;
or R₁₁ and R₁₂ or R₁₂ and R₁₃ may form together with the carbon atoms they are attached to C1-C5 alkyl substituted or unsubstituted, saturated or unsaturated 5 and 6 membered rings containing C, O and/or N atoms.

5. A fragrance composition comprising at least one compound of formula (I) according to claim 3.

6. A consumer product comprising at least one compound of formula (I) according to claim 3 and a consumer product base.

7. A method to release a phenolic fragrant compound of formula (II), the method comprising exposing a compound of formula (I) as defined in claim 3 to ambient air in the presence or absence of light.

8. A method of making a compound of formula (I) according to claim 3, comprising the steps of:
a) providing a phenolic fragrant compound HX, which is a compound of formula (II) 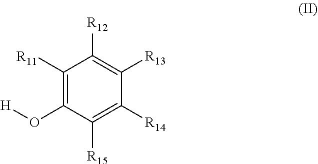
wherein
R₁₁ is selected from the group consisting of H, C1-C5 alkyl, OH, OMe, OEt, O-n-Pr, O-iso-Pr, hexanoyl, heptanoyl, octanoyl, propanoyl, 3-phenylpropanoyl, 5-methylhexanoyl, Cl, Br, phenyl, and CO₂R₁₉, wherein R₁₉ is selected from the group consisting of H, Me, Et, 2-methylpropyl, hexan-2-yl, 2-isopropoxyethyl, 3-methylbut-2-en-1-yl, n-pentyl, hexyl, benzyl, cyclohexyl, cis-3-hexenyl, 2-ethylhexyl, 3-methylhex-2-en-1-yl, 2-hept-4-enyl, 1-(3-methyl-2-hexenyl), (CH₂)₂-phenyl, and para-tolyl;
R₁₂ is selected from the group consisting of H, OH, C1-C5 alkyl, C2-C5 alkenyl, and C1-C5 alkoxy;
R₁₃ is selected from the group consisting of H, branched or linear C1-C5 alkyl, branched or linear C2-C5 alkenyl, CHO, CO₂H, CO₂Me, CO₂Et, C(O)Me, C(O)Et, (CH₂)₂C(O)CH₃;
(4-methyl-3,6-dihydro-2H-pyran-2-yl), CH₂—O-Me, CH₂—O-Et, CH₂—O-iPr, CH₂—OH, Cl, propanoyl, and CO₂R₁₉, wherein R₁₉ is selected from the group consisting of H, Me, Et, 2-methylpropyl, hexan-2-yl, 2-isopropoxyethyl, 3-methylbut-2-en-1-yl, n-pentyl, hexyl, benzyl, cyclohexyl, cis-3-hexenyl, 2-ethylhexyl, 3-methylhex-2-en-1-yl, 2-hept-4-enyl, 1-(3-methyl-2-hexenyl), (CH₂)₂-phenyl, and para-tolyl
R₁₄ is selected from the group consisting of H, OH, C1-C5 alkyl, C2-C5 alkenyl, and C1-C5 alkoxy;
R₁₅ is selected from the group consisting of H, C1-C5 alkyl, and Br;
or R₁₁ and R₁₂ or R₁₂ and R₁₃ may form together with the carbon atoms they are attached to C1-C5 alkyl substituted or unsubstituted, saturated or unsaturated 5 and 6 membered rings containing C, O and/or N atoms; and
b) reacting it with an unsaturated alkylhalide or an unsaturated alcohol.

9. A method to confer, enhance, improve or modify the hedonic properties of a fragrance composition or a consumer product, which method comprises adding to said composition or consumer product at least one compound of formula (I) as defined in claim 3.
